(19) United States
(12) Patent Application Publication
(10) Pub. No.: US 2015/0340627 A1
(43) Pub. Date: Nov. 26, 2015

(54) MATERIALS FOR ELECTRONIC DEVICES

(71) Applicant: MERCK PATENT GMBH, Darmstadt (DE)

(72) Inventors: Anja JATSCH, Frankfurt Am Main (DE); Christof PFLUMM, Darmstadt (DE); Amir Hossain PARHAM, Frankfurt Am Main (DE); Thomas EBERLE, Landau (DE); Philipp STOESSEL, Frankfurt Am Main (DE); Jonas Valentin KROEBER, Frankfurt Am Main (DE); Rouven LINGE, Darmstadt (DE)

(21) Appl. No.: 14/758,978

(22) PCT Filed: Dec. 10, 2013
(86) PCT No.: PCT/EP2013/003722

§ 371 Date: Jul. 2, 2015

(30) Foreign Application Priority Data
Jan. 3, 2013 (EP) 13000013.6

(51) Int. Cl. H01L 51/00; C07D 209/86; C07D 401/04; C08G 61/12; C07D 403/12; C07D 403/14; C07D 401/14; C07D 209/94; C07D 487/04

(52) U.S. Cl. CPC H01L 51/0072; C07D 209/94; C07D 209/86; C07D 401/04; C07D 487/04; C07D 403/12; C07D 403/14; C07D 401/14; H01L 51/0067; H01L 51/0052; H01L 51/0058; H01L 51/006; H01L 51/0061; C08G 61/124; C08G 2261/3241; C08G 2261/3221; C08G 2261/312; C08G 2261/314; C08G 2261/3142; C08G 2261/95; C08G 2261/514; C08G 2261/3162; H01L 51/5024

(57) ABSTRACT

The present application relates to a compound of a formula (I), (II) or (III). The compound can be used in an electronic device, preferably an organic electronic device.

The present application relates to a compound of a formula (I), (II) or (III) which contains a carbazole group and an electron-deficient heteroaryl group. The compound can be used in an electronic device, preferably an organic electronic device. The present application furthermore relates to a process for the preparation of the compound.

Electronic devices in the sense of this application are taken to mean, in particular, so-called organic electronic devices, which comprise organic semiconductor materials as functional materials. They are again taken to mean, in particular, organic electroluminescent devices (OLEDs) and other electronic devices which are mentioned below in the detailed description of the invention.

The precise structure of OLEDs is described, inter alia, in U.S. Pat. No. 4,539,507, U.S. Pat. No. 5,151,629, EP 0676461 and WO 98/27136. In general, the term OLED is taken to mean electronic devices which comprise at least one organic material and emit light on application of an electrical voltage.

In the case of electronic devices, in particular OLEDs, there is great interest in improving the performance data, in particular lifetime and efficiency and operating voltage. An important role is played here by organic emitter layers, in particular the matrix materials present therein, and organic layers having electron-transporting function.

In order to achieve this technical object, there is a continuous search for novel materials which are suitable for use as matrix materials in emitting layers, in particular phosphorescent emitting layers. Furthermore, materials having electron-transporting properties for use in electron-transporting layers are being sought.

Phosphorescent emitting layers in the sense of the present application are organic layers which comprise at least one phosphorescent emitting compound (phosphorescent dopant).

In accordance with the present application, the term phosphorescent emitters encompasses compounds in the case of which the light emission takes place through a spin-forbidden transition, for example a transition from an excited triplet state or a state having a higher spin quantum number, such as a quintet state.

A matrix material in a system comprising a matrix material and a dopant is taken to mean the component whose proportion in the mixture is the greater. Correspondingly, a dopant in a system comprising a matrix material and a dopant is taken to mean the component whose proportion in the mixture is the smaller.

In accordance with the prior art, carbazole derivatives, such as, for example, bis(carbazolyl)biphenyl, or carbazole compounds or indenocarbazole compounds, such as, for example, in accordance with WO 2005/039246, US 2005/0069729, JP 2004/288381, EP 1205527 or WO 2008/086851, are frequently used as matrix materials for phosphorescent emitters.

Triazine compounds, for example in accordance with WO 2010/015306, WO 2007/063754 or WO 2008/056746, are likewise used in this function.

The prior art furthermore discloses compounds in which a carbazole group or indenocarbazole group is bonded to a triazine group, for example in WO 2011/057706, WO 2010/136109 or WO 2011/000455.

However, there continues to be a need for improvement over the compounds known from the prior art, in particular in the aspects operating voltage and power efficiency of devices comprising the compounds.

Surprisingly, it has now been found that excellent values for operating voltage and power efficiency can be achieved with compounds which contain a carbazole group or indenocarbazole group which are connected to a donor-substituted electron-deficient six-membered heteroaromatic ring via a linker group on the N atom.

The present application thus relates to a compound of a formula (I), (II) or (III)

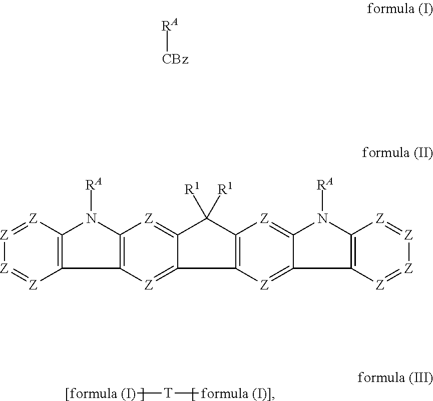

where:
Cbz is a carbazole group which is optionally substituted by one or more radicals R¹ and which may be extended by means of one or more condensed-on indeno groups to form an indenocarbazole, and in which one or more aromatic groups ═C(R¹)— or ═C(H)— may be replaced by ═N—, and which is bonded to the group R^{A} via the carbazole nitrogen atom;
[formula (I)] is on each occurrence, identically or differently, any desired unit of the formula (1), where the group T may be bonded to this unit at any desired position;
Z is on each occurrence, identically or differently, CR¹ or N;
R¹ is on each occurrence, identically or differently, H, D, F, C(═O)R², CN, Si(R²)₃, N(R²)₂, P(═O)(R²)₂, S(═O)R², S(═O)₂R², a straight-chain alkyl or alkoxy group having 1 to 20 C atoms or a branched or cyclic alkyl or alkoxy group having 3 to 20 C atoms or an alkenyl or alkynyl group

having 2 to 20 C atoms, where the above-mentioned groups may each be substituted by one or more radicals R² and where one or more CH₂ groups in the above-mentioned groups may be replaced by —R²C═CR²—, —C≡C—, Si(R²)₂, C═O, C═NR², —C(═O)O—, —C(═O)NR²—, NR², P(═O)(R²), —O—, —S—, SO or SO₂, or an aromatic or heteroaromatic ring system having 5 to 30 aromatic ring atoms, which may in each case be substituted by one or more radicals R², or an aryloxy or heteroaryloxy group having 5 to 30 aromatic ring atoms, which may be substituted by one or more radicals R², where two or more radicals R¹ may be linked to one another and may form a ring;
R^{A} is a group of the formula (A)

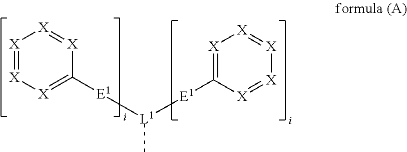

where the dashed line denotes the bond to the remainder of the formula, or R^{A} is equal to R¹, where at least one group R^{A} per formula unit of the formula (I) or (II) conforms to the formula (A);
L¹ is an aromatic or heteroaromatic ring system having 6 to 30 aromatic ring atoms, which may be substituted by one or more radicals R¹;
E¹ is on each occurrence, identically or differently, O, S, or NAr¹;
X is on each occurrence, identically or differently, N or CR^{x}, where at least one group X per six-membered ring is equal to N;
   i is on each occurrence, identically or differently, 0 or 1, where at least one index i per group of the formula (A) is equal to 1;
R^{x} is on each occurrence, identically or differently, H, D, F, C(═O)R², CN, Si(R²)₃, S(═O)R², S(═O)₂R², a straight-chain alkyl group having 1 to 20 C atoms or a branched or cyclic alkyl group having 3 to 20 C atoms or an alkenyl or alkynyl group having 2 to 20 C atoms, where the above-mentioned groups may each be substituted by one or more radicals R² and where one or more CH₂ groups in the above-mentioned groups may be replaced by —R²C═CR²—, —C≡C—, Si(R²)₂, C═O, C═NR², —C(═O)O—, —C(═O)NR²—, NR², P(═O)(R²), —O—, —S—, SO or SO₂, or an aromatic or heteroaromatic ring system having 5 to 30 aromatic ring atoms, which may in each case be substituted by one or more radicals R²;
R² is on each occurrence, identically or differently, H, D, F, C(═O)R³, CN, Si(R³)₃, N(R³)₂, P(═O)(R³)₂, S(═O)R³, S(═O)₂R³, a straight-chain alkyl or alkoxy group having 1 to 20 C atoms or a branched or cyclic alkyl or alkoxy group having 3 to 20 C atoms or an alkenyl or alkynyl group having 2 to 20 C atoms, where the above-mentioned groups may each be substituted by one or more radicals R³ and where one or more CH₂ groups in the above-mentioned groups may be replaced by —R³C═CR³—, —C≡C—, Si(R³)₂, C═O, C═NR³, —C(═O)O—, —C(═O)NR³—, NR³, P(═O)(R³), —O—, —S—, SO or SO₂, or an aromatic or heteroaromatic ring system having 5 to 30 aromatic ring atoms, which may in each case be substituted by one or more radicals R³, or an aryloxy or heteroaryloxy group having 5 to 30 aromatic ring atoms, which may be substituted by one or more radicals R³, where two or more radicals R² may be linked to one another and may form a ring;
R³ is on each occurrence, identically or differently, H, D, F or an aliphatic, aromatic or heteroaromatic organic radical having 1 to 20 C atoms, in which, in addition, one or more H atoms may be replaced by D or F; two or more substituents R³ here may be linked to one another and form a ring;
Ar¹ is an aromatic ring system having 6 to 30 aromatic ring atoms, which may be substituted by one or more radicals R¹;
T is a single bond or an aromatic or heteroaromatic ring system having 6 to 30 aromatic ring atoms, which may be substituted by one or more radicals R¹.

For the purposes of the present application, the definition that the group Cbz is a carbazole group, which may be extended by means of indeno groups to form an indenocarbazole, is taken to mean that indeno groups may be condensed onto one or both of the six-membered rings of the carbazole. If indeno groups are present, one or two are preferably present. If two indeno groups are present, they are preferably not both bonded to the same six-membered ring of the carbazole.

An indeno group here is taken to mean the following structure:

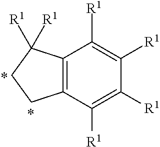

Condensation of the indeno group is taken to mean that it shares two ring atoms with two ring atoms of the six-membered ring of the carbazole. These two ring atoms are preferably the ring atoms labelled with *.

The condensation of indeno groups onto the carbazole group in the group Cbz preferably takes place in positions 2 and 3 and/or positions 6 and 7, where the numbering of the positions on the carbazole, as generally customary, takes place as shown below. However, it may also take place in positions 1 and 2, 3 and 4, 5 and 6 and/or 7 and 8.

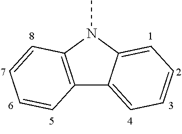

An illustrative carbazole group Cbz onto which an indeno group is condensed is the following:

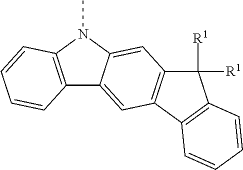

where the group may be substituted by radicals R¹ at all free positions, and where the dashed line denotes the bond to the group L¹.

An illustrative carbazole group Cbz onto which two indeno groups are condensed is the following:

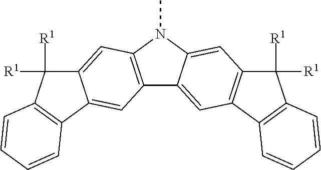

where the group may be substituted by radicals R¹ at all free positions, and where the dashed line denotes the bond to the group L¹.

General definitions of chemical groups in accordance with the present application follow.

An aryl group in the sense of this invention contains 6 to 60 aromatic ring atoms; a heteroaryl group in the sense of this invention contains 5 to 60 aromatic ring atoms, at least one of which is a heteroatom. The heteroatoms are preferably selected from N, O and S. This represents the basic definition. If other preferences are indicated in the description of the present invention, for example with respect to the number of aromatic ring atoms or the heteroatoms present, these apply.

An aryl group or heteroaryl group here is taken to mean either a simple aromatic ring, i.e. benzene, or a simple heteroaromatic ring, for example pyridine, pyrimidine or thiophene, or a condensed (annellated) aromatic or heteroaromatic polycycle, for example naphthalene, phenanthrene, quinoline or carbazole. A condensed (annellated) aromatic or heteroaromatic polycycle in the sense of the present application consists of two or more simple aromatic or heteroaromatic rings condensed with one another.

An aryl or heteroaryl group, which may in each case be substituted by the above-mentioned radicals and which may be linked to the aromatic or heteroaromatic ring system via any desired positions, is taken to mean, in particular, groups derived from benzene, naphthalene, anthracene, phenanthrene, pyrene, dihydropyrene, chrysene, perylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, pyrazine, phenazine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

An aryloxy group in accordance with the definition of the present invention is taken to mean an aryl group, as defined above, which is bonded via an oxygen atom. An analogous definition applies to heteroaryloxy groups.

An aromatic ring system in the sense of this invention contains 6 to 60 C atoms in the ring system. A heteroaromatic ring system in the sense of this invention contains 5 to 60 aromatic ring atoms, at least one of which is a heteroatom. The heteroatoms are preferably selected from N, O and/or S. An aromatic or heteroaromatic ring system in the sense of this invention is intended to be taken to mean a system which does not necessarily contain only aryl or heteroaryl groups, but instead in which, in addition, a plurality of aryl or heteroaryl groups may be connected by a non-aromatic unit (preferably less than 10% of the atoms other than H), such as, for example, an sp³-hybridised C, Si, N or O atom, an sp²-hybridised C or N atom or an sp-hybridised C atom. Thus, for example, systems such as 9,9′-spirobifluorene, 9,9′-diarylfluorene, triarylamine, diaryl ether, stilbene, etc., are also intended to be taken to be aromatic ring systems in the sense of this invention, as are systems in which two or more aryl groups are connected, for example, by a linear or cyclic alkyl, alkenyl or alkynyl group or by a silyl group. Furthermore, systems in which two or more aryl or heteroaryl groups are linked to one another via single bonds are also taken to be aromatic or heteroaromatic ring systems in the sense of this invention, such as, for example, systems such as biphenyl, terphenyl or diphenyltriazine.

An aromatic or heteroaromatic ring system having 5-60 aromatic ring atoms, which may in each case also be substituted by radicals as defined above and which may be linked to the aromatic or heteroaromatic group via any desired positions, is taken to mean, in particular, groups derived from benzene, naphthalene, anthracene, benzanthracene, phenanthrene, benzophenanthrene, pyrene, chrysene, perylene, fluoranthene, naphthacene, pentacene, benzopyrene, biphenyl, biphenylene, terphenyl, terphenylene, quaterphenyl, fluorene, spirobifluorene, dihydrophenanthrene, dihydropyrene, tetrahydropyrene, cis- or trans-indenofluorene, truxene, isotruxene, spiro-truxene, spiroisotruxene, furan, benzofuran, isobenzofuran, dibenzofuran, thiophene, benzothiophene, isobenzothiophene, dibenzothiophene, pyrrole, indole, isoindole, carbazole, indolocarbazole, indenocarbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubin, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole, or combinations of these groups.

For the purposes of the present invention, a straight-chain alkyl group having 1 to 40 C atoms or a branched or cyclic alkyl group having 3 to 40 C atoms or an alkenyl or alkynyl group having 2 to 40 C atoms, in which, in addition, individual H atoms or CH₂ groups may be substituted by the groups mentioned above under the definition of the radicals, is preferably taken to mean the radicals methyl, ethyl, n-propyl, i-propyl, n-butyl, i-butyl, s-butyl, t-butyl, 2-methylbutyl, n-pentyl, s-pentyl, cyclopentyl, neopentyl, n-hexyl, cyclohexyl, neohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl. An alkoxy or thioalkyl group having 1 to 40 C atoms is preferably taken to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy, 2,2,2-trifluoroethoxy, methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio.

The formulation that two or more radicals may form a ring with one another is, for the purposes of the present application, intended to be taken to mean, inter alia, that the two radicals are linked to one another by a chemical bond. This is illustrated by the following scheme:

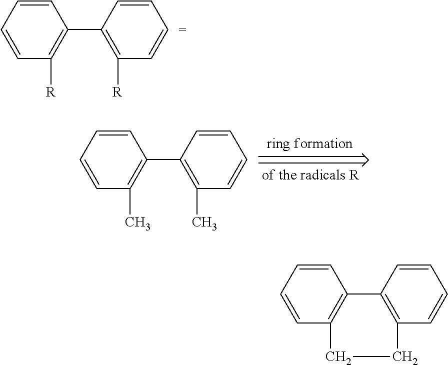

Furthermore, however, the above-mentioned formulation is also intended to be taken to mean that, in the case where one of the two radicals represents hydrogen, the second radical is bonded at the position to which the hydrogen atom was bonded, with formation of a ring. This is illustrated by the following scheme:

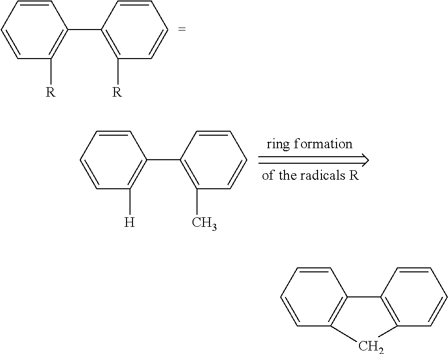

The compound of the formula (I), (II) or (III) preferably contains no condensed aryl or heteroaryl groups having more than 14 aromatic ring atoms, particularly preferably no aryl or heteroaryl groups having more than 10 aromatic ring atoms.

It is preferred in accordance with the invention for one index i per formula (A) to be equal to one and for the other index i to be equal to zero.

Furthermore preferably, two or three groups X per six-membered ring are equal to N.

Furthermore preferably, groups X which represent N are not adjacent in a six-membered ring.

Ar¹ is furthermore preferably selected from an aromatic ring system having 6 to 18 aromatic ring atoms, which may be substituted by one or more radicals R¹. Ar¹ is particularly preferably selected from phenyl, biphenyl, terphenyl, naphthyl, fluorenyl or spirobifluorenyl, each of which is optionally substituted by radicals R¹.

Groups of the formula (A) preferably conform to one of the following formulae (A-1) to (A-8)

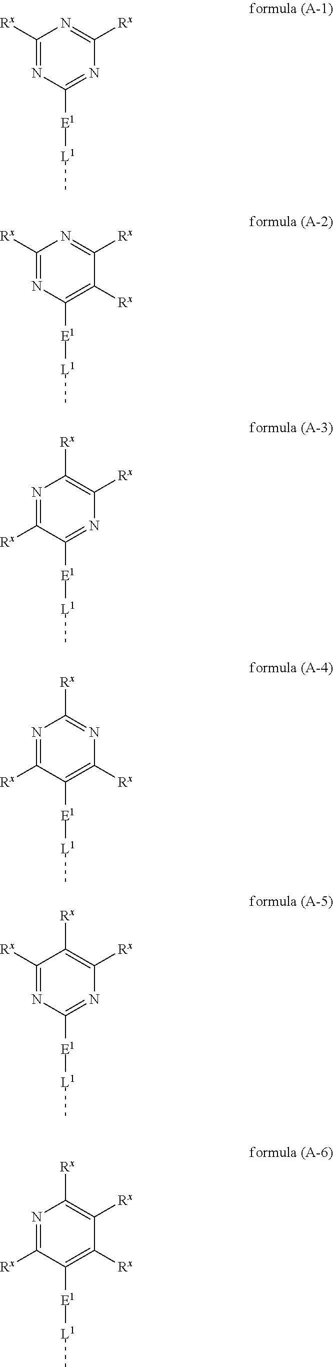

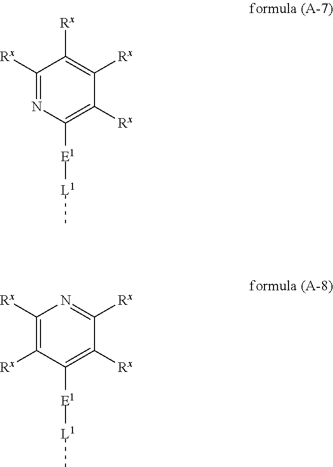

where the groups occurring are as defined above, and where the dashed line denotes the bond to the remainder of the formula.

For groups of the formulae (A-1) to (A-8), the embodiments indicated as preferred in the present application relating to the groups L¹, E¹ and R^{X} are likewise regarded as preferred.

E¹ is furthermore preferably selected identically on each occurrence. E¹ is furthermore preferably on each occurrence, identically or differently, O or S, particularly preferably O.

L¹ is furthermore preferably an aromatic or heteroaromatic ring system having 6 to 24 aromatic ring atoms, particularly preferably an aromatic ring system having 6 to 24 aromatic ring atoms, where the ring systems may be substituted by one or more radicals R¹.

The group L¹ furthermore preferably contains at least one meta- or ortho-phenylene group, which may optionally be substituted by one or more radicals R¹.

Very particularly preferred groups L¹ are selected from groups of the following formulae (L-1) to (L-18)

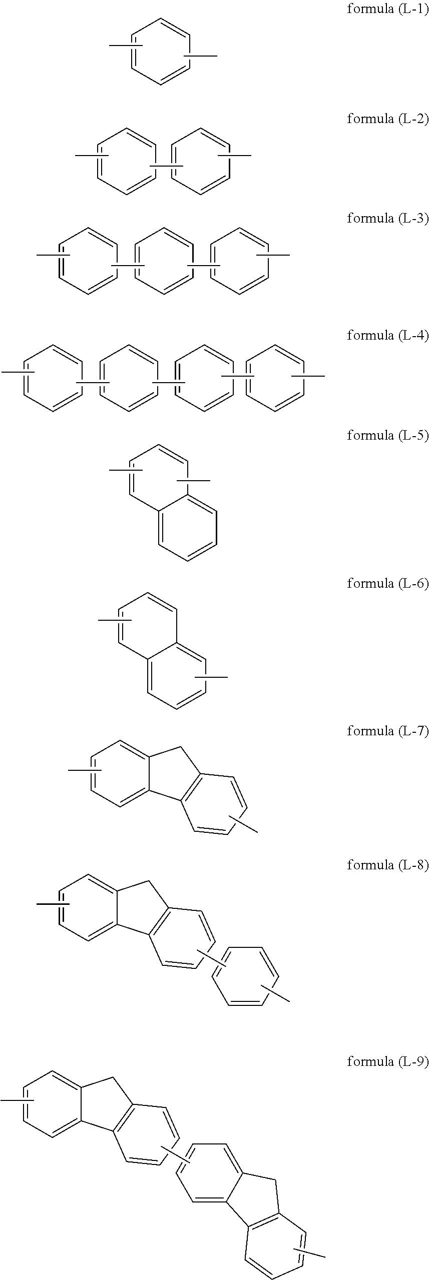

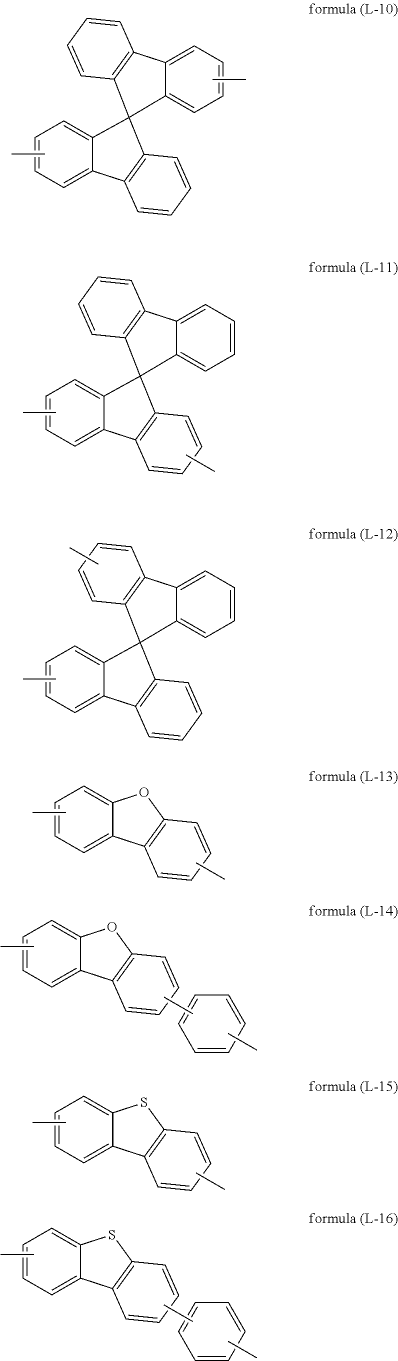

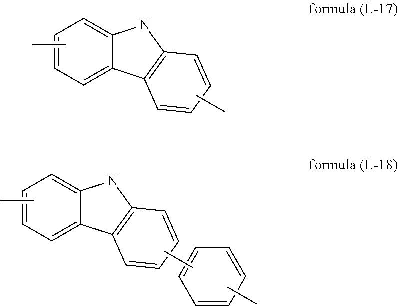

where the groups may be substituted by radicals R¹ at all free positions and where the dashed lines denote the bonds to the remainder of the compound in the case where the sum of the indices i is equal to 1 and only one group E¹ is present. In the case where the sum of the indices i is equal to 2, so that two groups E¹ are present, preferably both groups E¹ are bonded to the same aryl group. Correspondingly modified groups of the formulae (L-1) to (L-18) which correspondingly contain three dashed lines which denote the bonds to the remainder of the formula instead of two dashed lines should then be called into play.

Furthermore preferably, no groups ═C(R¹)— or ═C(H)— in the group Cbz have been replaced by ═N—.

It is furthermore generally regarded as preferred for not more than three groups Z per six-membered ring to be equal to N, particularly preferably not more than two groups Z. Furthermore preferably, not more than two adjacent groups Z are equal to N. Furthermore preferably, Z is equal to CR¹.

Preferred groups Cbz conform to the following formulae (Cbz-1) to (Cbz-3)

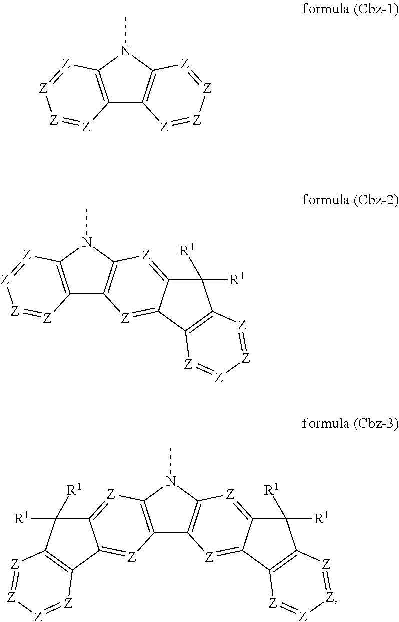

where the dashed line denotes the bond to the group R^{A}, and where the groups occurring are as defined above.

R¹ is preferably on each occurrence, identically or differently, H, D, F, C(═O)R², CN, Si(R²)₃, a straight-chain alkyl or alkoxy group having 1 to 10 C atoms or a branched or cyclic alkyl or alkoxy group having 3 to 10 C atoms, where the above-mentioned groups may each be substituted by one or more radicals R² and where one or more CH₂ groups in the above-mentioned groups may be replaced by —C≡C—, —R³C═CR³—, Si(R³)₂ or C═O, or an aromatic or heteroaromatic ring system having 5 to 20 aromatic ring atoms, which may in each case be substituted by one or more radicals R², where two or more radicals R¹ may be linked to one another and may form a ring.

R¹ which is bonded to the methylene group of an indeno group which is a constituent of a group Cbz or of the indenocarbazole group of formula (II) is preferably selected from a straight-chain alkyl group having 1 to 10 C atoms, or a branched or cyclic alkyl group having 3 to 10 C atoms, where the above-mentioned groups may each be substituted by one or more radicals R², or the two radicals R¹ which are bonded to the same methylene group are linked to one another and form an alkyl ring with the methylene group, where the alkyl ring may in each case be substituted by one or more radicals R².

Preferred embodiments of alkyl rings which are formed by two radicals R¹ on a methylene group —C(R¹)₂— in a group Cbz which represents an indenocarbazole group are selected from the following formulae (C-1) to (C-8)

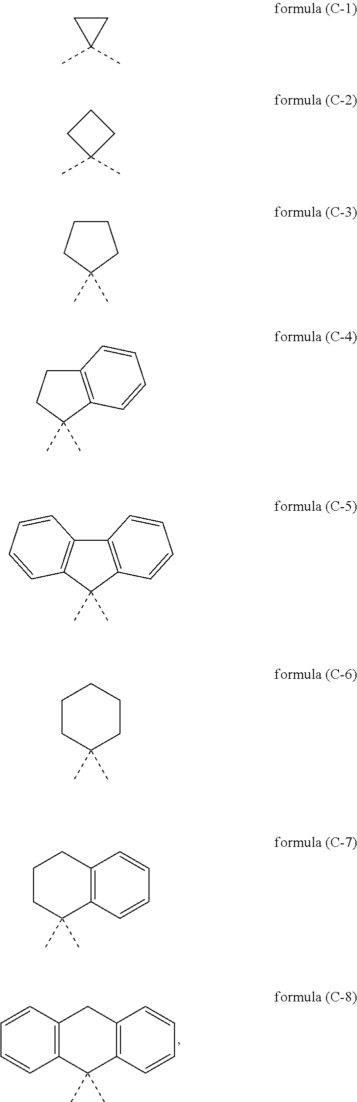

each of which may be substituted by radicals R² at the free positions.

For formula (II), both groups R^{A} are preferably each groups of the formula (A). For formula (II), however, one R^{A} may also be a group of the formula (A) and the other R^{A} is equal to R¹. For formula (I), R^{A} can by definition not be equal to R¹, but instead must conform to formula (A).

R² is furthermore preferably on each occurrence, identically or differently, H, D, F, C(═O)R³, CN, Si(R³)₃, a straight-chain alkyl or alkoxy group having 1 to 10 C atoms or a branched or cyclic alkyl or alkoxy group having 3 to 10 C atoms, where the above-mentioned groups may each be substituted by one or more radicals R³ and where one or more CH₂ groups in the above-mentioned groups may be replaced by —C≡C—, —R³C═CR³—, Si(R³)₂ or C═O, or an aromatic or heteroaromatic ring system having 5 to 20 aromatic ring atoms, which may in each case be substituted by one or more radicals R³, where two or more radicals R² may be linked to one another and may form a ring.

R^{x} is furthermore preferably on each occurrence, identically or differently, H, D, F, CN, a straight-chain alkyl group having 1 to 10 C atoms or a branched or cyclic alkyl group having 3 to 10 C atoms or an alkenyl or alkynyl group having 2 to 10 C atoms, where the above-mentioned groups may each be substituted by one or more radicals R² and where one or more CH₂ groups in the above-mentioned groups may be replaced by —R²C═CR²—, —C≡C—, Si(R²)₂ or C═O, or an aromatic or heteroaromatic ring system having 5 to 20 aromatic ring atoms, which may in each case be substituted by one or more radicals R².

Preferred compounds of the formula (1) conform to one of the following formulae (I-1) to (I-24)

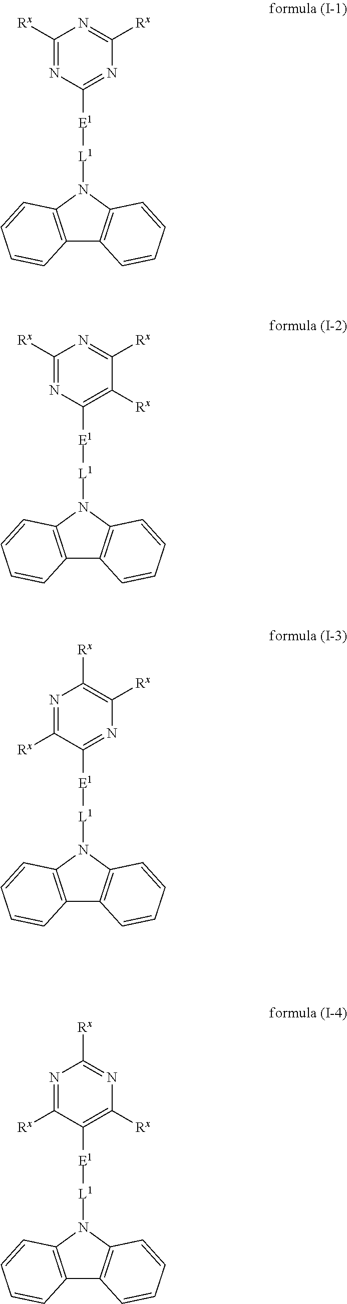

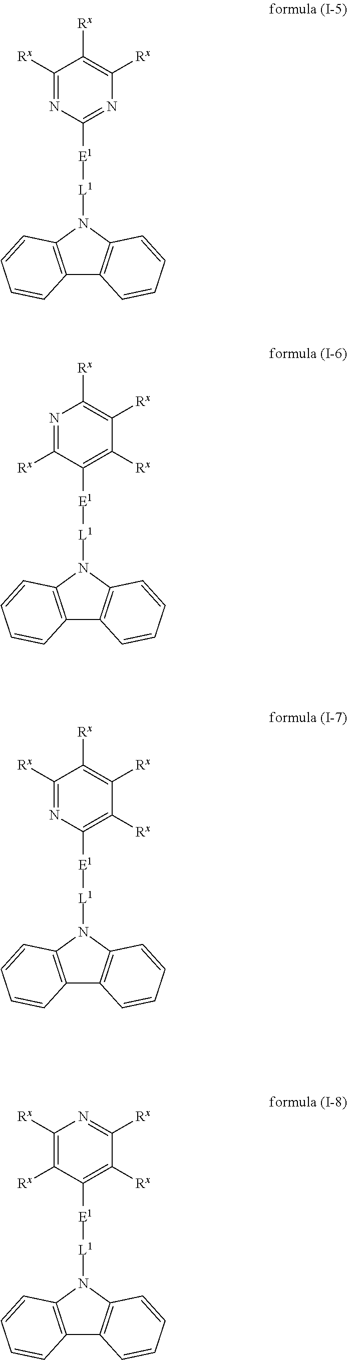

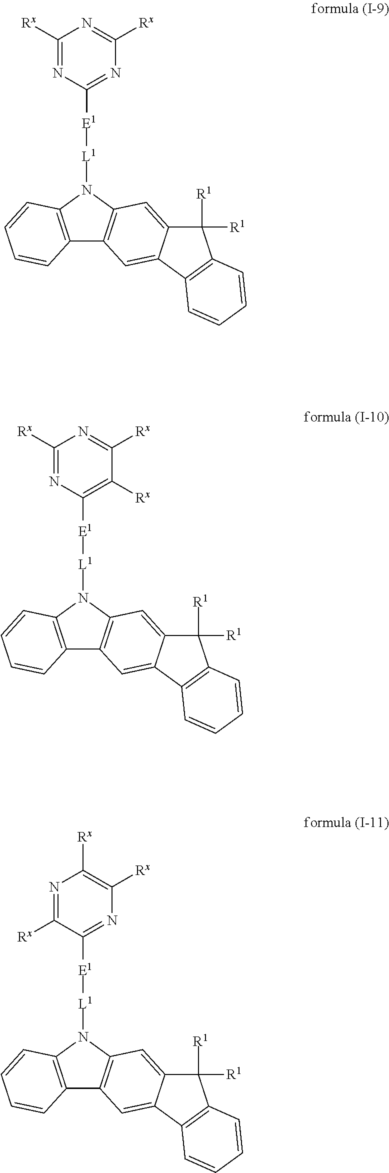

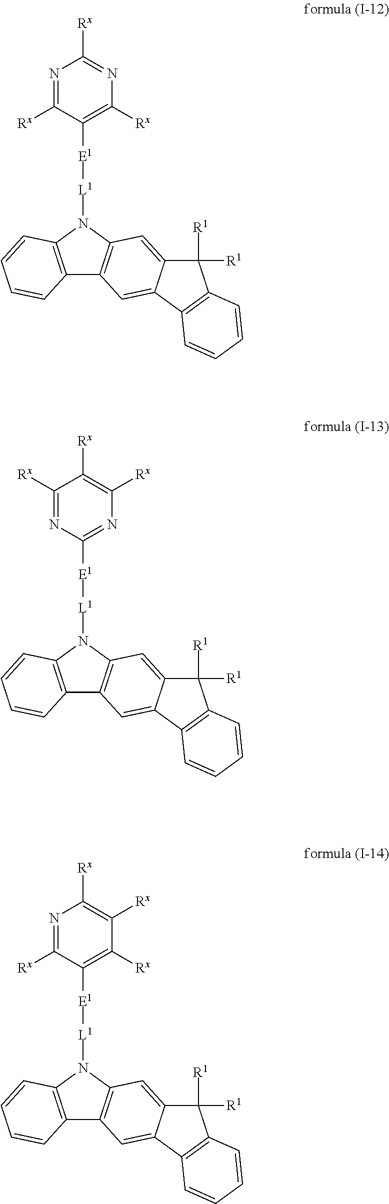

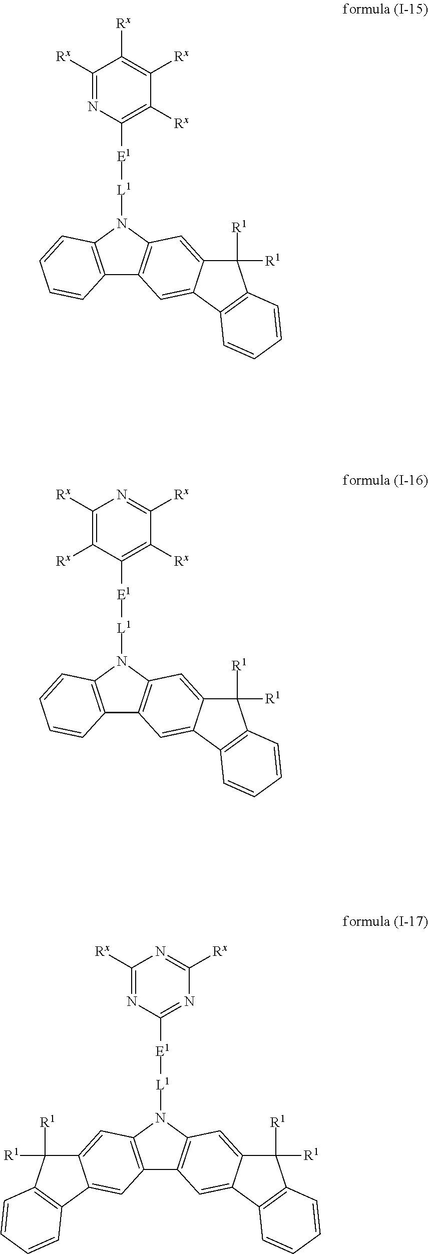

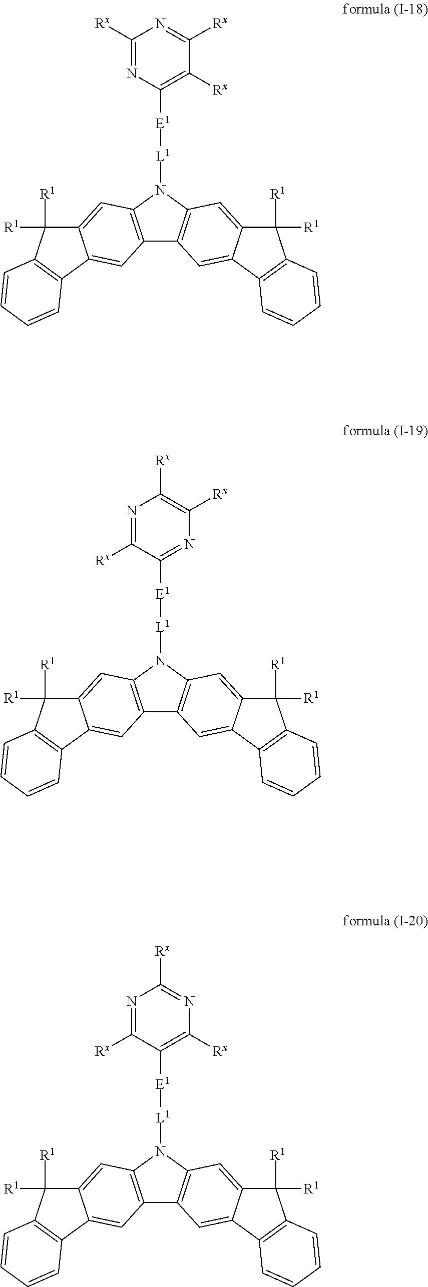

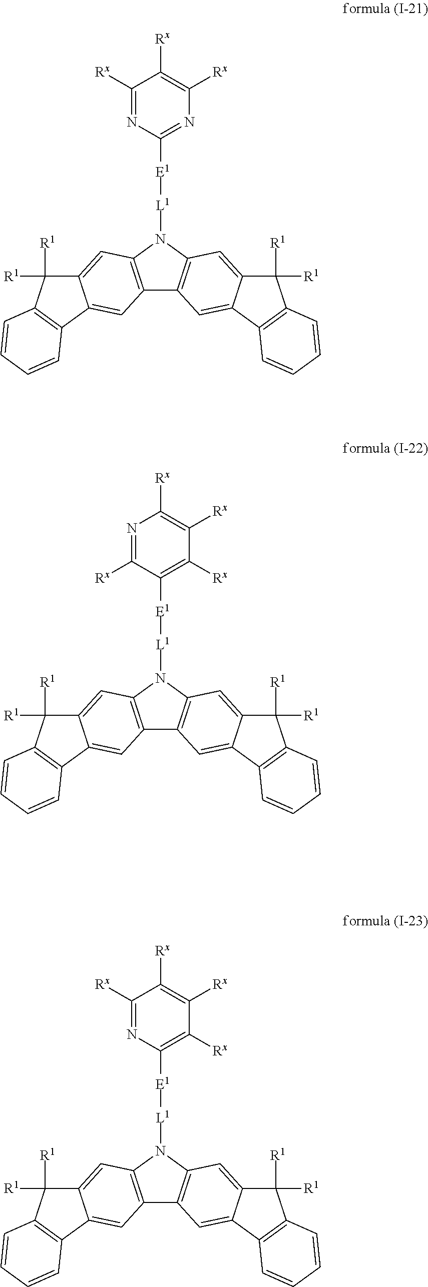

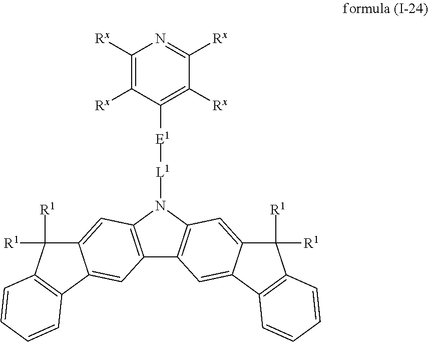

where the compounds may each be substituted by radicals R¹ at all free positions on the groups Cbz, and where the groups occurring are as defined above.

For compounds of the formulae (I-1) to (I-24), it is preferred for the groups occurring to be defined in accordance with their preferred embodiments.

E¹ is especially preferably selected from O and S.

L¹ is furthermore especially preferably selected from an aromatic or heteroaromatic ring system having 6 to 24 aromatic ring atoms, particularly preferably an aromatic ring system having 6 to 24 aromatic ring atoms, where the ring systems may be substituted by one or more radicals R¹. R¹ is very particularly preferably selected from groups of the formulae (L-1) to (L-18), as defined above.

Preferred compounds of the formula (II) conform to one of the following formulae (II-1) to (II-8)

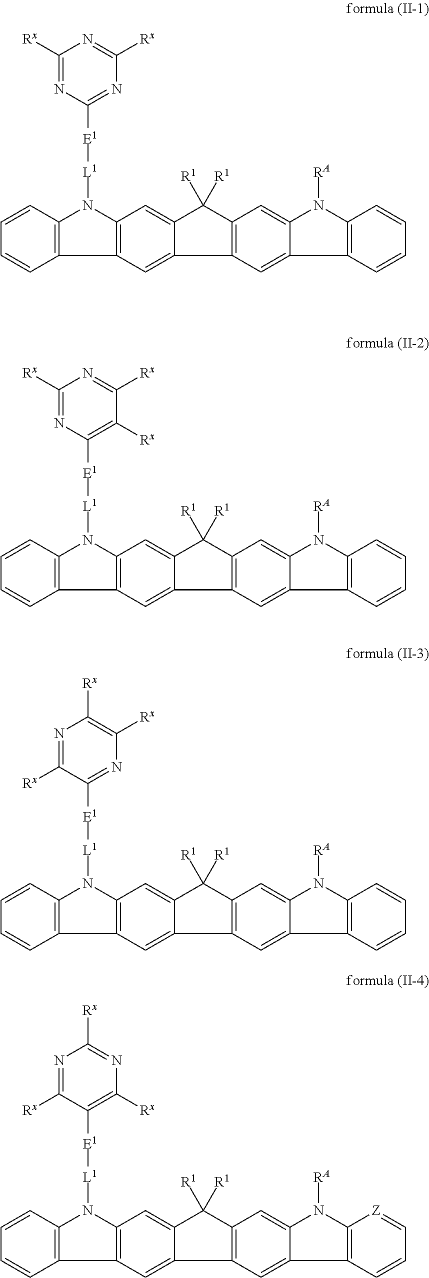

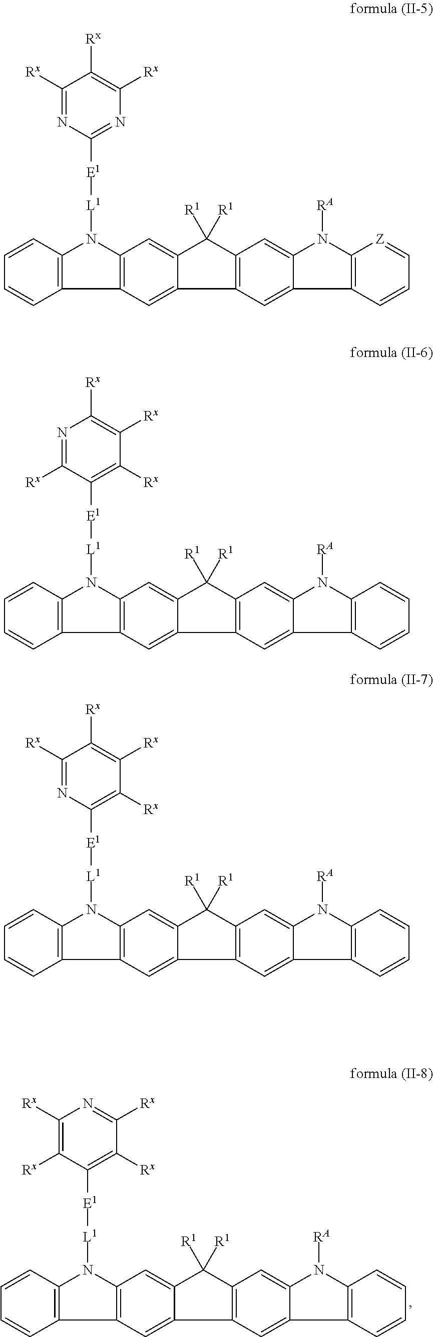

where the compounds may each be substituted by radicals R¹ at all positions depicted as unsubstituted,
and where the groups occurring are as defined above,
and where, in particular, R^{A} may be equal to R¹ or equal to a group of the formula (A).

For compounds of the formulae (II-1) to (II-8), it is preferred for the groups occurring to be defined in accordance with their preferred embodiments.

R^{A} in formulae (II-1) to (II-8) is preferably a group of the formula (A). R^{A} is particularly preferably selected in such a way that the two groups bonded to the carbazole nitrogen atoms are identical.

E¹ is especially preferably selected from O and S.

Furthermore, L¹ is especially preferably selected from an aromatic or heteroaromatic ring system having 6 to 24 aromatic ring atoms, particularly preferably an aromatic ring system having 6 to 24 aromatic ring atoms, where the ring systems may be substituted by one or more radicals R¹. R¹ is very particularly preferably selected from groups of the formulae (L-1) to (L-18), as defined above.

For compounds of the formula (III), T is in general preferably a single bond or an aromatic or heteroaromatic ring system having 6 to 24 aromatic ring atoms, which may be substituted by one or more radicals R¹. T is particularly preferably a single bond.

For compounds of the formula (III), the groups of the unit of the formula (I) furthermore generally preferably correspond to their preferred embodiments indicated above. The units of the formula (I) especially preferably correspond to the preferred embodiments of the formulae (I-1) to (I-21) indicated above.

Furthermore, the group T is preferably in each case bonded to the group Cbz of the unit of the formula (I).

Furthermore, the units of the formula (I) in compounds of the formula (III) are preferably each selected identically.

Particularly preferred embodiments of compounds of the formula (III) conform to the following formulae (III-1) to (III-5)

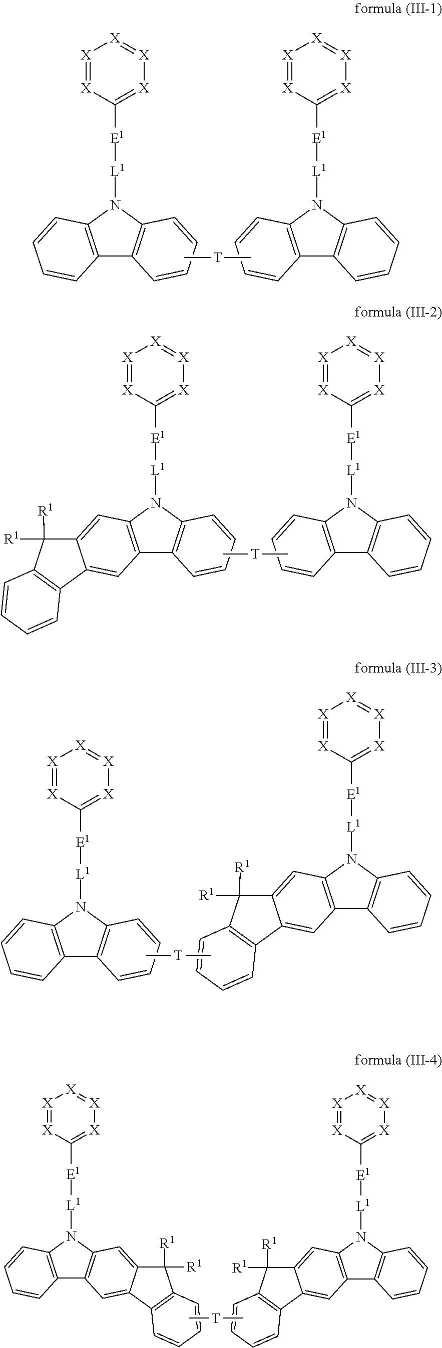

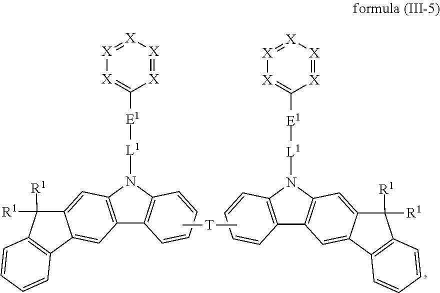

where the groups occurring are as defined above.

For compounds of the formulae (III-1) to (III-5), the groups occurring are preferably defined in accordance with their preferred embodiments.

E¹ is especially preferably selected from O and S.

Furthermore, L¹ is especially preferably selected from an aromatic or heteroaromatic ring system having 6 to 24 aromatic ring atoms, particularly preferably an aromatic ring system having 6 to 24 aromatic ring atoms, where the ring systems may be substituted by one or more radicals R¹. R¹ is very particularly preferably selected from groups of the formulae (L-1) to (L-18), as defined above.

Furthermore, for compounds of the formulae (III-1) to (III-5), T is especially preferably a single bond or an aromatic or heteroaromatic ring system having 6 to 24 aromatic ring atoms, which may be substituted by one or more radicals R¹. T in compounds of the formula (III-1) to (III-5) is particularly preferably a single bond.

Examples of compounds according to the invention are shown below:

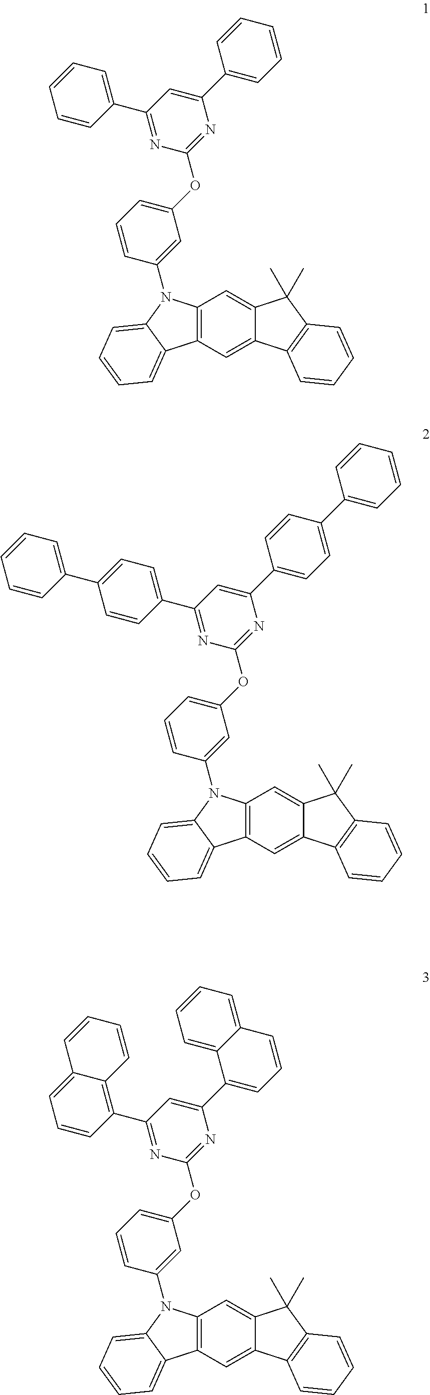 [SMILES: CC1(C)c2ccccc2-c2cc3c4ccccc4n(-c4cccc(Oc5nc(-c6ccccc6)cc(-c6ccccc6)n5)c4)c3cc21 ; CC1(C)c2ccccc2-c2cc3c4ccccc4n(-c4cccc(Oc5nc(-c6ccc(-c7ccccc7)cc6)cc(-c6ccc(-c7ccccc7)cc6)n5)c4)c3cc21 ; CC1(C)c2ccccc2-c2cc3c4ccccc4n(-c4cccc(Oc5nc(-c6cccc7ccccc67)cc(-c6cccc7ccccc67)n5)c4)c3cc21]

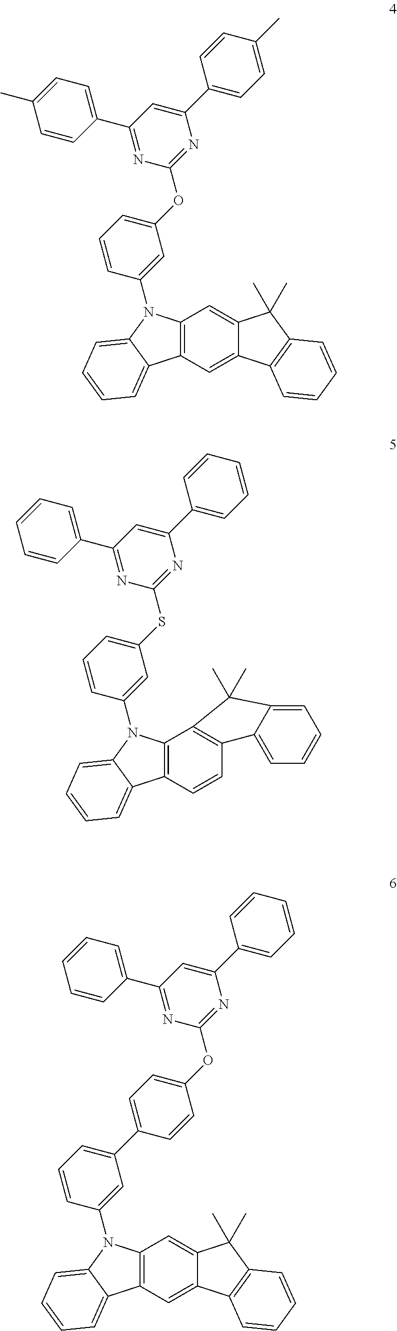 [SMILES: Cc1ccc(-c2cc(-c3ccc(C)cc3)nc(Oc3cccc(-n4c5ccccc5c5cc6c(cc54)C(C)(C)c4ccccc4-6)c3)n2)cc1 ; CC1(C)c2ccccc2-c2ccc3c4ccccc4n(-c4cccc(Sc5nc(-c6ccccc6)cc(-c6ccccc6)n5)c4)c3c21 ; CC1(C)c2ccccc2-c2cc3c4ccccc4n(-c4cccc(-c5ccc(Oc6nc(-c7ccccc7)cc(-c7ccccc7)n6)cc5)c4)c3cc21]

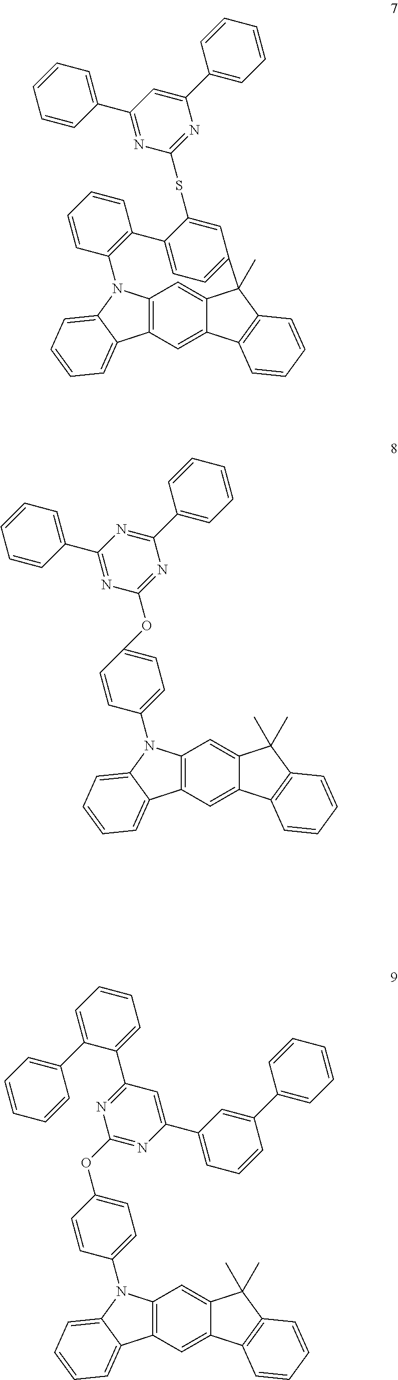 [SMILES: CC12c3ccc(c(Sc4nc(-c5ccccc5)cc(-c5ccccc5)n4)c3)-c3ccccc3-n3c4ccccc4c4cc(c1cc43)-c1ccccc12 ; CC1(C)c2ccccc2-c2cc3c4ccccc4n(-c4ccc(Oc5nc(-c6ccccc6)nc(-c6ccccc6)n5)cc4)c3cc21 ; CC1(C)c2ccccc2-c2cc3c4ccccc4n(-c4ccc(Oc5nc(-c6cccc(-c7ccccc7)c6)cc(-c6ccccc6-c6ccccc6)n5)cc4)c3cc21]

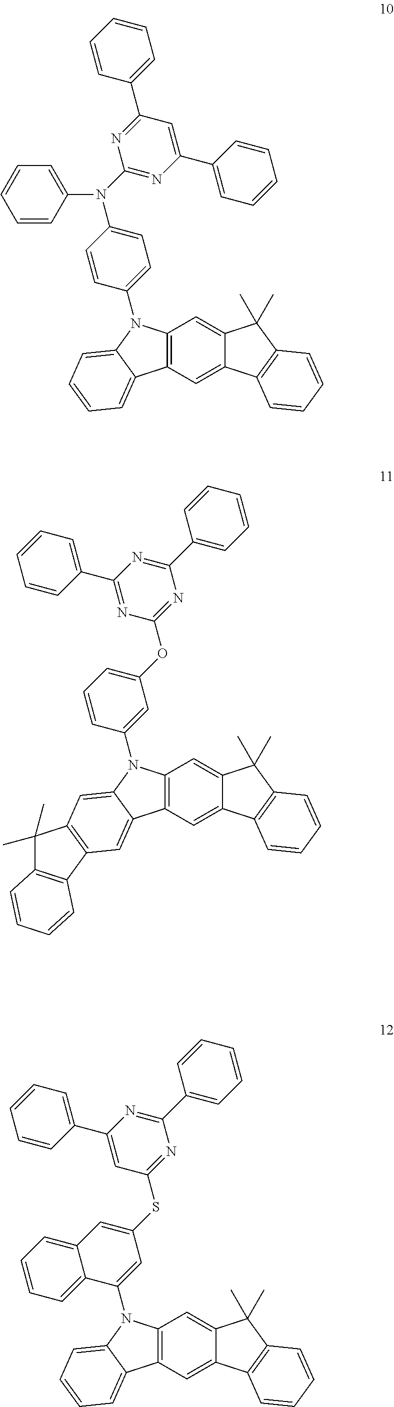 [SMILES: CC1(C)c2ccccc2-c2cc3c4ccccc4n(-c4ccc(N(c5ccccc5)c5nc(-c6ccccc6)cc(-c6ccccc6)n5)cc4)c3cc21 ; CC1(C)c2ccccc2-c2cc3c4cc5c(cc4n(-c4cccc(Oc6nc(-c7ccccc7)nc(-c7ccccc7)n6)c4)c3cc21)C(C)(C)c1ccccc1-5 ; CC1(C)c2ccccc2-c2cc3c4ccccc4n(-c4cc(Sc5cc(-c6ccccc6)nc(-c6ccccc6)n5)cc5ccccc45)c3cc21]

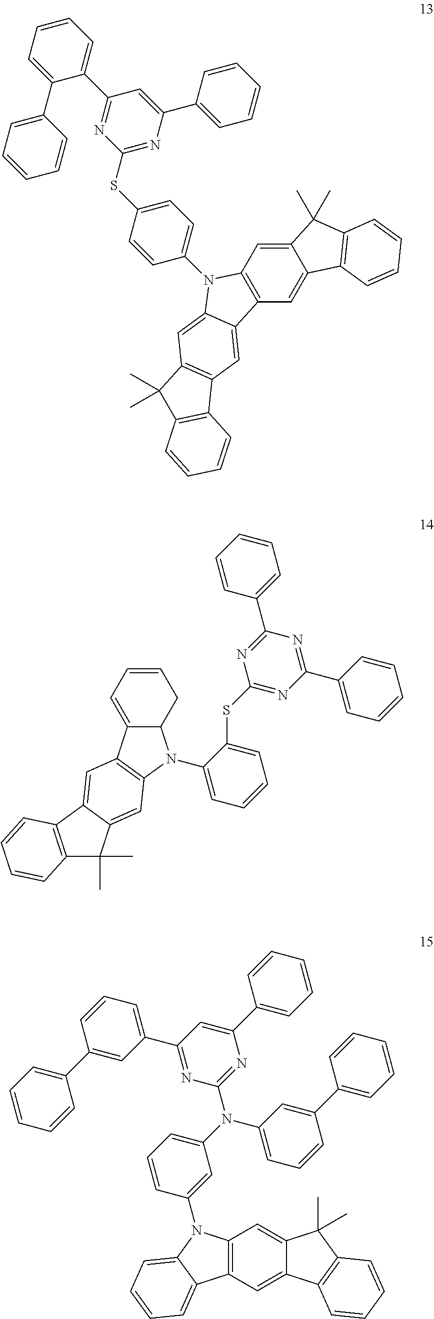 [SMILES: CC1(C)c2ccccc2-c2cc3c4cc5c(cc4n(-c4ccc(Sc6nc(-c7ccccc7)cc(-c7ccccc7-c7ccccc7)n6)cc4)c3cc21)C(C)(C)c1ccccc1-5 ; CC1(C)c2ccccc2-c2cc3c(cc21)N(c1ccccc1Sc1nc(-c2ccccc2)nc(-c2ccccc2)n1)C1CC=CC=C31 ; CC1(C)c2ccccc2-c2cc3c4ccccc4n(-c4cccc(N(c5cccc(-c6ccccc6)c5)c5nc(-c6ccccc6)cc(-c6cccc(-c7ccccc7)c6)n5)c4)c3cc21]

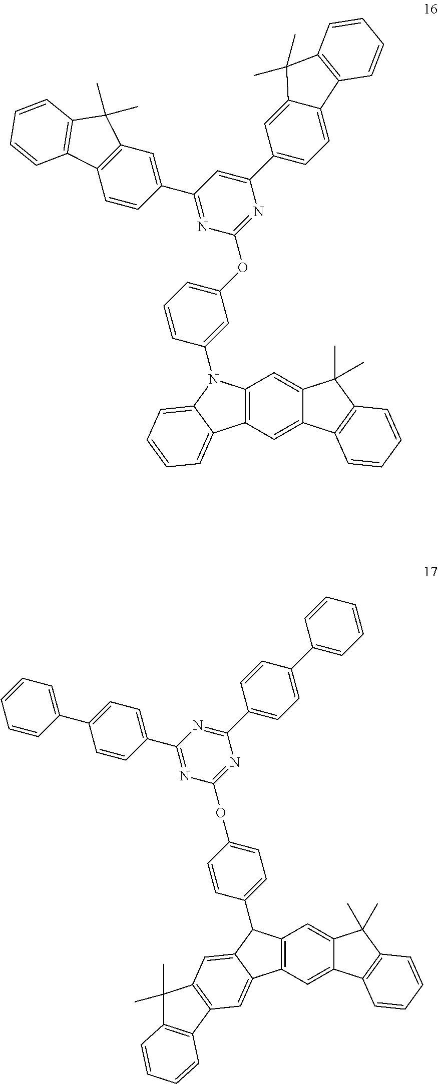 [SMILES: CC1(C)c2ccccc2-c2ccc(-c3cc(-c4ccc5c(c4)C(C)(C)c4ccccc4-5)nc(Oc4cccc(-n5c6ccccc6c6cc7c(cc65)C(C)(C)c5ccccc5-7)c4)n3)cc21 ; CC1(C)c2ccccc2-c2cc3c(cc21)C(c1ccc(Oc2nc(-c4ccc(-c5ccccc5)cc4)nc(-c4ccc(-c5ccccc5)cc4)n2)cc1)c1cc2c(cc1-3)-c1ccccc1C2(C)C]

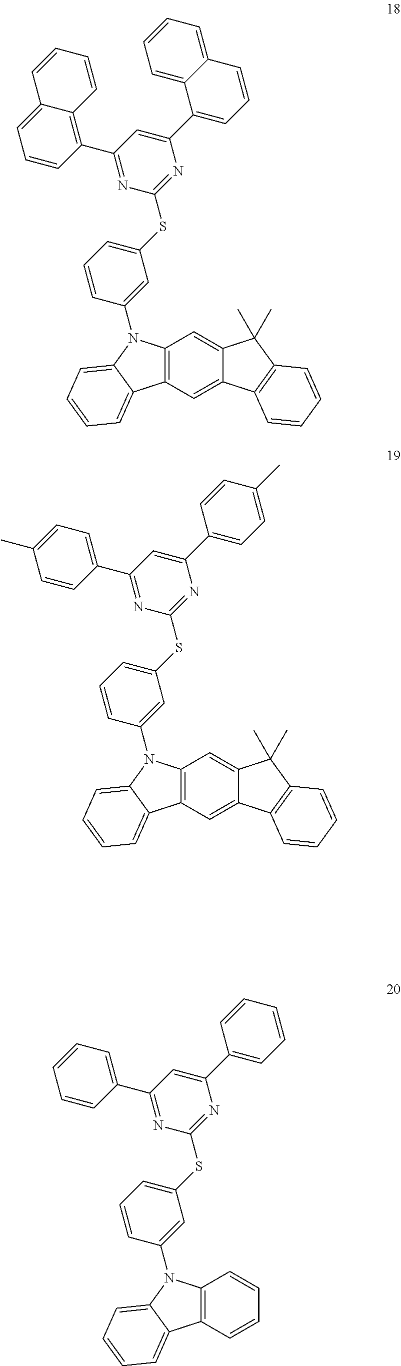

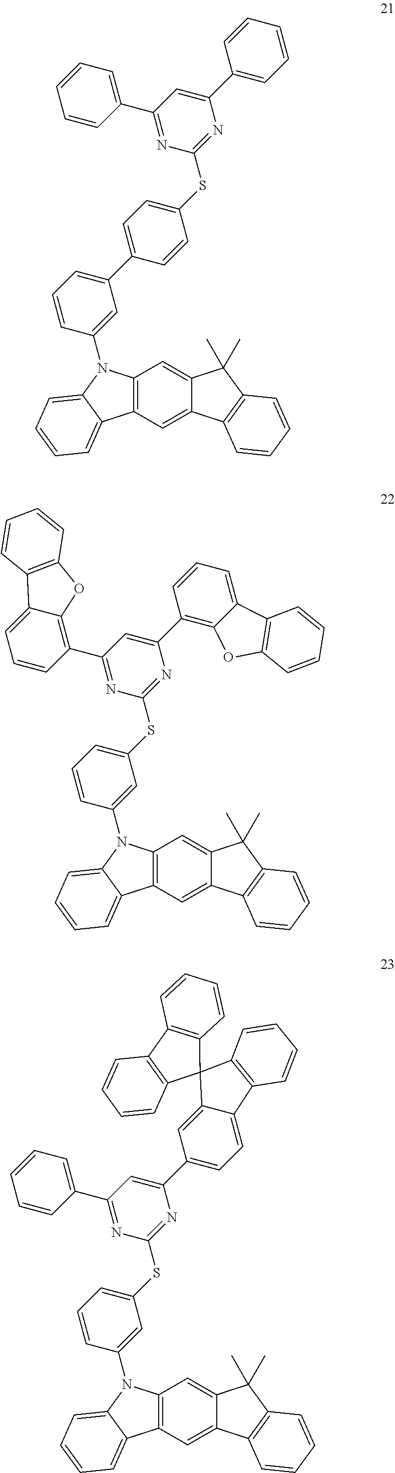 [SMILES: CC1(C)c2ccccc2-c2cc3c4ccccc4n(-c4cccc(-c5ccc(Sc6nc(-c7ccccc7)cc(-c7ccccc7)n6)cc5)c4)c3cc21 ; CC1(C)c2ccccc2-c2cc3c4ccccc4n(-c4cccc(Sc5nc(-c6cccc7c6oc6ccccc67)cc(-c6cccc7c6oc6ccccc67)n5)c4)c3cc21 ; CC1(C)c2ccccc2-c2cc3c4ccccc4n(-c4cccc(Sc5nc(-c6ccccc6)cc(-c6ccc7c(c6)C6(c8ccccc8-c8ccccc86)c6ccccc6-7)n5)c4)c3cc21]

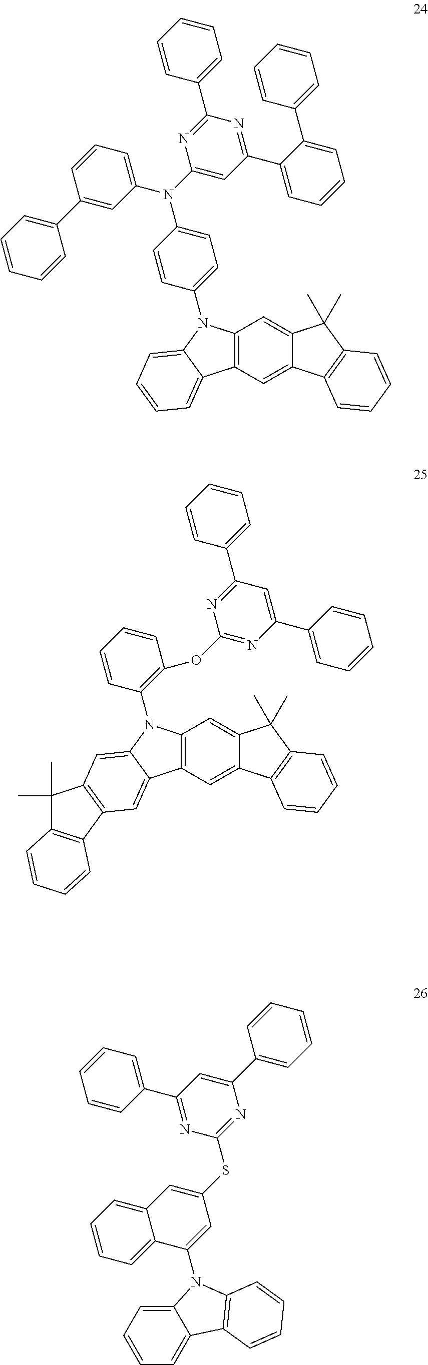 [SMILES: CC1(C)c2ccccc2-c2cc3c4ccccc4n(-c4ccc(N(c5cccc(-c6ccccc6)c5)c5cc(-c6ccccc6-c6ccccc6)nc(-c6ccccc6)n5)cc4)c3cc21 ; CC1(C)c2ccccc2-c2cc3c4cc5c(cc4n(-c4ccccc4Oc4nc(-c6ccccc6)cc(-c6ccccc6)n4)c3cc21)C(C)(C)c1ccccc1-5 ; c1ccc(-c2cc(-c3ccccc3)nc(Sc3cc(-n4c5ccccc5c5ccccc54)c4ccccc4c3)n2)cc1]

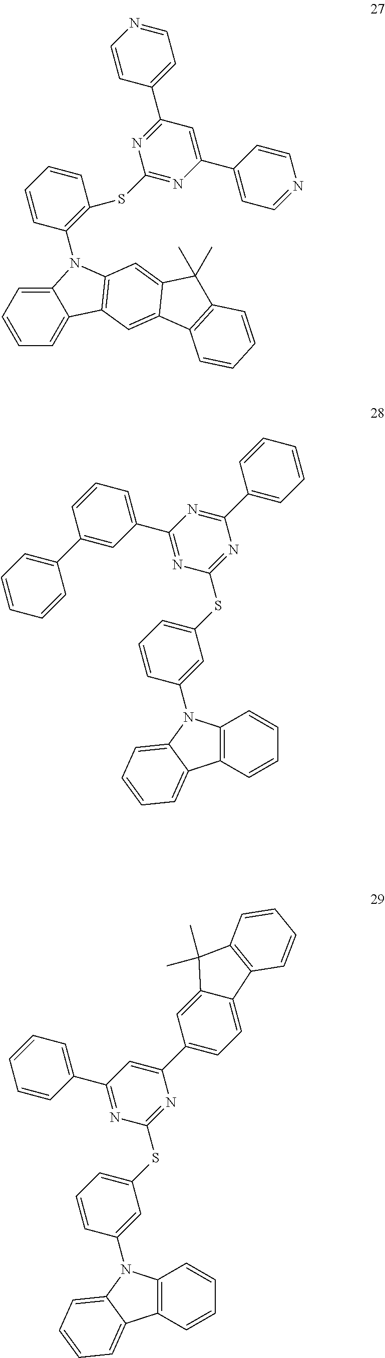 [SMILES: CC1(C)c2ccccc2-c2cc3c4ccccc4n(-c4ccccc4Sc4nc(-c5ccncc5)cc(-c5ccncc5)n4)c3cc21 ; c1ccc(-c2cccc(-c3nc(Sc4cccc(-n5c6ccccc6c6ccccc65)c4)nc(-c4ccccc4)n3)c2)cc1 ; CC1(C)c2ccccc2-c2ccc(-c3cc(-c4ccccc4)nc(Sc4cccc(-n5c6ccccc6c6ccccc65)c4)n3)cc21]

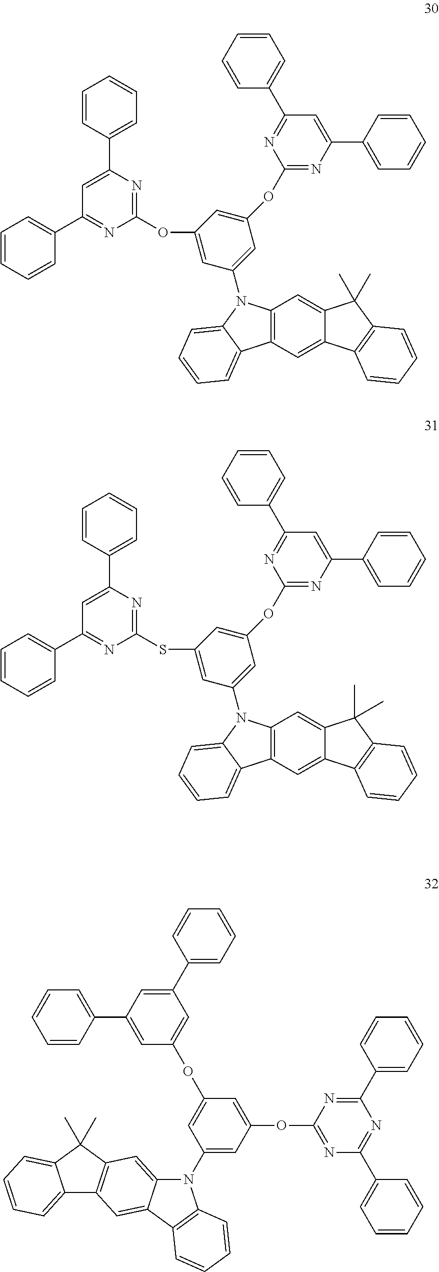 [SMILES: CC1(C)c2ccccc2-c2cc3c4ccccc4n(-c4cc(Oc5nc(-c6ccccc6)cc(-c6ccccc6)n5)cc(Oc5nc(-c6ccccc6)cc(-c6ccccc6)n5)c4)c3cc21 ; CC1(C)c2ccccc2-c2cc3c4ccccc4n(-c4cc(Oc5nc(-c6ccccc6)cc(-c6ccccc6)n5)cc(Sc5nc(-c6ccccc6)cc(-c6ccccc6)n5)c4)c3cc21 ; CC1(C)c2ccccc2-c2cc3c4ccccc4n(-c4cc(Oc5cc(-c6ccccc6)cc(-c6ccccc6)c5)cc(Oc5nc(-c6ccccc6)nc(-c6ccccc6)n5)c4)c3cc21]

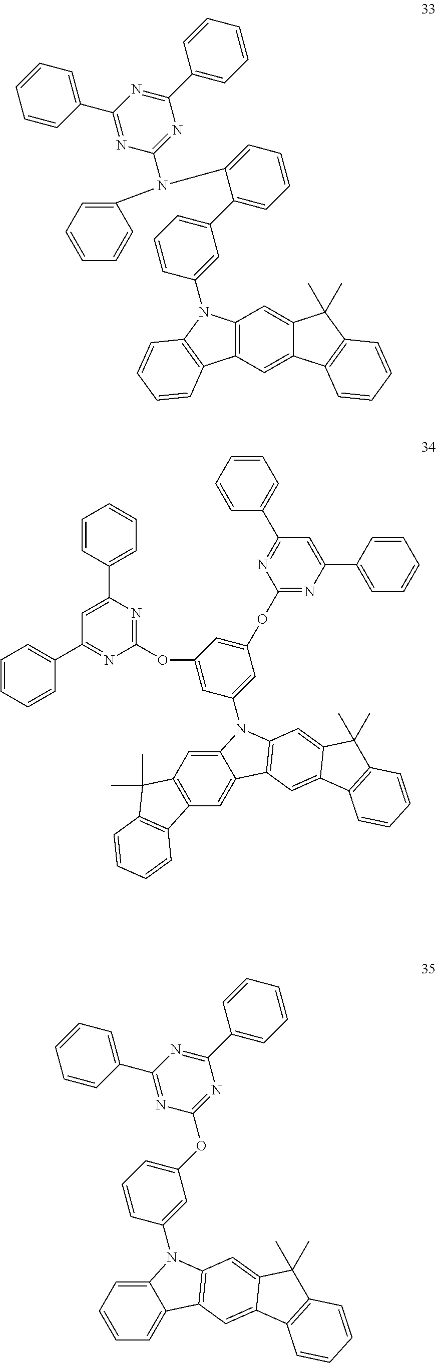 [SMILES: CC1(C)c2ccccc2-c2cc3c4ccccc4n(-c4cccc(-c5ccccc5N(c5ccccc5)c5nc(-c6ccccc6)nc(-c6ccccc6)n5)c4)c3cc21 ; CC1(C)c2ccccc2-c2cc3c4cc5c(cc4n(-c4cc(Oc6nc(-c7ccccc7)cc(-c7ccccc7)n6)cc(Oc6nc(-c7ccccc7)cc(-c7ccccc7)n6)c4)c3cc21)C(C)(C)c1ccccc1-5 ; CC1(C)c2ccccc2-c2cc3c4ccccc4n(-c4cccc(Oc5nc(-c6ccccc6)nc(-c6ccccc6)n5)c4)c3cc21]

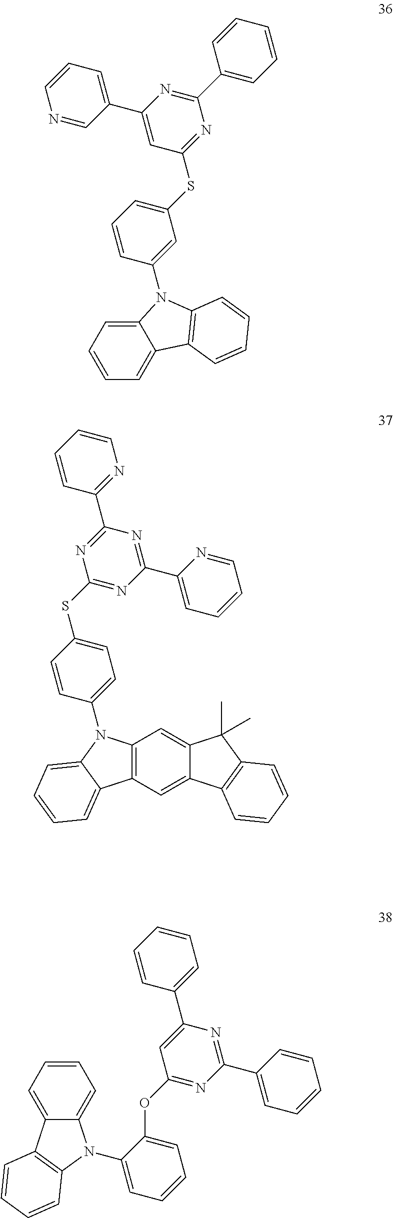 [SMILES: c1ccc(-c2nc(Sc3cccc(-n4c5ccccc5c5ccccc54)c3)cc(-c3cccnc3)n2)cc1 ; CC1(C)c2ccccc2-c2cc3c4ccccc4n(-c4ccc(Sc5nc(-c6ccccn6)nc(-c6ccccn6)n5)cc4)c3cc21 ; c1ccc(-c2cc(Oc3ccccc3-n3c4ccccc4c4ccccc43)nc(-c3ccccc3)n2)cc1]

The compounds according to the invention can be prepared by known organochemical synthesis processes. These include, for example, the Hartwig-Buchwald coupling, the Suzuki coupling, halogenation reactions and nucleophilic substitution reactions on electron-deficient aromatic compounds.

Illustrative processes for the preparation of the compounds according to the invention are presented below. The processes shown are particularly suitable for the preparation of compounds according to the invention. However, alternative processes are conceivable and possibly to be preferred in certain cases. Correspondingly, the person skilled in the art will be able to modify the processes shown below within the bounds of his general expert knowledge.

Scheme 1 shows the synthesis of compounds according to the invention which contain an oxygen- or sulfur-functionalised electron-deficient heteroaryl group. To this end, firstly a protected oxygen- or sulfur-functionalised linker is coupled to a carbazole derivative in a Buchwald coupling. After deprotection, this linker is reacted with an electron-deficient heteroaromatic compound in a substitution reaction. This gives a compound according to the invention, which, however, can be functionalised and modified further.

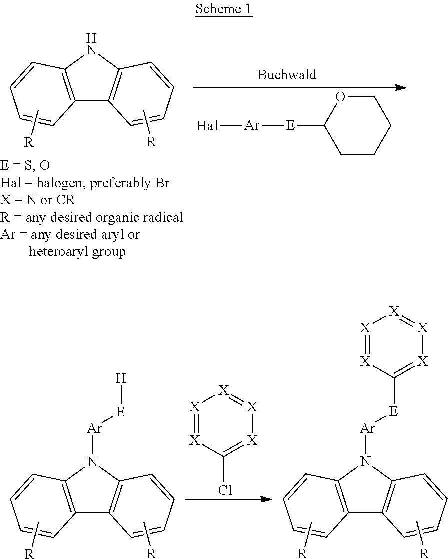

Scheme 2 shows the synthesis of compounds which contain a nitrogen-functionalised electron-deficient heteroaryl group. To this end, firstly a halogen-substituted linker is coupled to the carbazole derivative in a Buchwald coupling. In a second Buchwald reaction, the product is subsequently coupled to an amino group which has been functionalised by means of an electron-deficient heteroaryl group. This gives a compound according to the invention, which, however, can be functionalised and modified further.

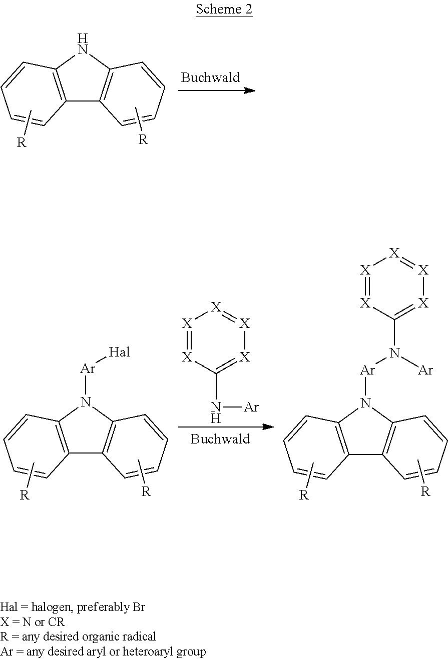

The preparation of dimeric compounds of the formula (III) can be carried out starting from corresponding modified starting compounds. Alternatively, monomeric compounds obtained in accordance with Scheme 1 or 2 can be functionalised and coupled or extended to give dimeric compounds.

It is furthermore noted that it is also possible to use indenocarbazoles as starting materials instead of the carbazoles shown, in which case the corresponding indenocarbazole derivatives are obtained as compounds according to the invention.

In summary, the invention furthermore relates to a process for the preparation of a compound of the formula (I), (II) or (III), characterised in that at least one transition metal-catalysed coupling reaction is employed.

The transition metal-catalysed coupling reaction is preferably a Hartwig-Buchwald coupling, which is particularly preferably carried out on the nitrogen atom of the carbazole derivative.

The electron-deficient heteroaryl group is furthermore preferably introduced by a Hartwig-Buchwald reaction if it is substituted by an amino group, and by a nucleophilic aromatic substitution reaction if it is substituted by an oxygen or sulfur in the compound of the formula (I), (II) or (III).

The compounds according to the invention described above, in particular compounds which are substituted by reactive leaving groups, such as bromine, iodine, chlorine, boronic acid or boronic acid ester, can be used as monomers for the production of corresponding oligomers, dendrimers or polymers. Suitable reactive leaving groups are, for example, bromine, iodine, chlorine, boronic acids, boronic acid esters, amines, alkenyl or alkynyl groups having a terminal C—C double bond or C—C triple bond, oxiranes, oxetanes, groups which undergo a cycloaddition, for example a 1,3-dipolar cycloaddition, such as, for example, dienes or azides, carboxylic acid derivatives, alcohols and silanes.

The invention therefore furthermore relates to oligomers, polymers or dendrimers containing one or more compounds of the formula (I), (II) or (III), where the bond(s) to the polymer, oligomer or dendrimer may be localised at any desired positions in formula (I), (II) or (III) which are substituted by R¹ or Rx. Depending on the linking of the compound of the formula (I), (II) or (III) the compound is a constituent of a side chain of the oligomer or polymer or a constituent of the main chain. An oligomer in the sense of this invention is taken to mean a compound which is built up from at least three monomer units. A polymer in the sense of the invention is taken to mean a compound which is built up from at least ten monomer units. The polymers, oligomers or dendrimers according to the invention may be conjugated, partially conjugated or non-conjugated. The oligomers or polymers according to the invention may be linear, branched or dendritic. In the structures linked in a linear manner, the units of the formula (I), (II) or (III) may be linked directly to one another or they may be linked to one another via a divalent group, for example via a substituted or unsubstituted alkylene group, via a heteroatom or via a divalent aromatic or heteroaromatic group. In branched and dendritic structures, for example, three or more units of the formula (I), (II) or (III) may be linked via a trivalent or polyvalent group, for example via a trivalent or polyvalent aromatic or heteroaromatic group, to form a branched or dendritic oligomer or polymer.

The same preferences as described above for compounds of the formula (I), (II) or (III) apply to the recurring units of the formula (I), (II) or (III) in oligomers, dendrimers and polymers.

For the preparation of the oligomers or polymers, the monomers according to the invention are homopolymerised or copolymerised with further monomers.

Suitable and preferred comonomers are selected from fluorenes (for example in accordance with EP 842208 or WO 00/22026), spirobifluorenes (for example in accordance with EP 707020, EP 894107 or WO 06/061181), paraphenylenes (for example in accordance with WO 1992/18552), carbazoles (for example in accordance with WO 04/070772 or WO 2004/113468), thiophenes (for example in accordance with EP 1028136), dihydrophenanthrenes (for example in accordance with WO 2005/014689 or WO 2007/006383), cis- and trans-indenofluorenes (for example in accordance with WO 2004/041901 or WO 2004/113412), ketones (for example in accordance with WO 2005/040302), phenanthrenes (for example in accordance with WO 2005/104264 or WO 2007/017066) or also a plurality of these units. The polymers, oligomers and dendrimers usually also contain further units, for example emitting (fluorescent or phosphorescent) units, such as, for example, vinyltriarylamines (for example in accordance with WO 2007/068325) or phosphorescent metal complexes (for example in accordance with WO 2006/003000), and/or charge-transport units, in particular those based on triarylamines.

The polymers, oligomers and dendrimers according to the invention have advantageous properties, in particular long lifetimes, high efficiencies and good colour coordinates.

The polymers and oligomers according to the invention are generally prepared by polymerisation of one or more types of monomer, at least one monomer of which results in recurring units of the formula (I), (II) or (III) in the polymer. Suitable polymerisation reactions are known to the person skilled in the art and are described in the literature. Particularly suitable and preferred polymerisation reactions which result in C—C or C—N links are the following:

(A) SUZUKI polymerisation;
(B) YAMAMOTO polymerisation;
(C) STILLE polymerisation; and
(D) HARTWIG-BUCHWALD polymerisation.

The way in which the polymerisation can be carried out by these methods and the way in which the polymers can then be separated off from the reaction medium and purified is known to the person skilled in the art and is described in detail in the literature, for example in WO 2003/048225, WO 2004/037887 and WO 2004/037887.

The present invention thus also relates to a process for the preparation of the polymers, oligomers and dendrimers according to the invention, which is characterised in that they are prepared by SUZUKI polymerisation, YAMAMOTO polymerisation, STILLE polymerisation or HARTWIG-BUCHWALD polymerisation. The dendrimers according to the invention can be prepared by processes known to the person skilled in the art or analogously thereto. Suitable processes are described in the literature, such as, for example, in Frechet, Jean M. J.; Hawker, Craig J., “Hyperbranched polyphenylene and hyperbranched polyesters: new soluble, three-dimensional, reactive polymers”, Reactive & Functional Polymers (1995), 26(1-3), 127-36; Janssen, H. M.; Meijer, E. W., “The synthesis and characterization of dendritic molecules”, Materials Science and Technology (1999), 20 (Synthesis of Polymers), 403-458; Tomalia, Donald A., “Dendrimer molecules”, Scientific American (1995), 272(5), 62-6; WO 2002/067343 A1 and WO 2005/026144 A1.

For the processing of the compounds according to the invention from the liquid phase, for example by spin coating or by printing processes, formulations of the compounds according to the invention are necessary. These formulations can be, for example, solutions, dispersions or emulsions. It may be preferred to use mixtures of two or more solvents for this purpose. Suitable and preferred solvents are, for example, toluene, anisole, o-, m- or p-xylene, methyl benzoate, mesitylene, tetralin, veratrol, THF, methyl-THF, THP, chlorobenzene, dioxane, phenoxytoluene, in particular 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, ca-terpineol, benzothiazole, butyl benzoate, cumene, cyclohexanol, cyclohexanone, cyclohexylbenzene, decalin, dodecyl-benzene, ethyl benzoate, indane, methyl benzoate, NMP, p-cymene, phenetol, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane or mixtures of these solvents.

The invention therefore furthermore relates to a formulation, in particular a solution, dispersion or emulsion, comprising at least one compound of the formula (I) or at least one polymer, oligomer or dendrimer containing at least one unit of the formula (I), and at least one solvent, preferably an organic solvent. The way in which solutions of this type can be prepared is known to the person skilled in the art and is described, for example, in WO 2002/072714, WO 2003/019694 and the literature cited therein.

The compounds according to the invention are suitable for use in electronic devices, in particular in organic electroluminescent devices (OLEDs). Depending, inter alia, on the substitution, the compounds can be employed in different functions and layers. The compounds are preferably employed as host materials, preferably as host materials for phosphorescent emitters, or as electron-transport materials.

The invention furthermore relates to the use of the compounds of the formula (I), (II) or (III) in electronic devices. The electronic devices here are preferably selected from the group consisting of organic integrated circuits (O-ICs), organic field-effect transistors (O-FETs), organic thin-film transistors (O-TFTs), organic light-emitting transistors (O-LETs), organic solar cells (O-SCs), organic optical detectors, organic photoreceptors, organic field-quench devices (O-FQDs), light-emitting electrochemical cells (LECs), organic laser diodes (O-lasers) and particularly preferably selected from organic electroluminescent devices (OLEDs).

The invention furthermore relates to an electronic device comprising anode, cathode and at least one organic layer, where the organic layer comprises at least one compound of the formula (I), (II) or (III). The electronic device here is preferably selected from the above-mentioned devices and is particularly preferably an organic electroluminescent device (OLED).

Apart from cathode, anode and the emitting layer, the organic electroluminescent device may also comprise further layers. These are selected, for example, from in each case one or more hole-injection layers, hole-transport layers, hole-blocking layers, electron-transport layers, electron-injection layers, electron-blocking layers, exciton-blocking layers, charge-generation layers (IDMC 2003, Taiwan; Session 21 OLED (5), T. Matsumoto, T. Nakada, J. Endo, K. Mori, N. Kawamura, A. Yokoi, J. Kido, Multiphoton Organic EL Device Having Charge Generation Layer), coupling-out layers and/or organic or inorganic p/n junctions. However, it should be pointed out that each of these layers does not necessarily have to be present and the choice of layers is always dependent on the compounds used and in particular also on whether the electroluminescent device is fluorescent or phosphorescent. The compounds preferably employed in the respective layers and functions are explicitly disclosed in later sections.

The sequence of the layers of the organic electroluminescent device is preferably as follows:

anode
hole-injection layer
hole-transport layer
optionally 1, 2 or 3 further hole-transport layers, preferably 2 further hole-transport layers
emitting layer
electron-transport layer
electron-injection layer
cathode.

It should again be pointed out here that not all the said layers have to be present, and/or that further layers may additionally be present.

The organic electroluminescent device according to the invention may comprise a plurality of emitting layers. These emission layers in this case particularly preferably have in total a plurality of emission maxima between 380 nm and 750 nm, resulting overall in white emission, i.e. various emitting compounds which are able to fluoresce or phosphoresce and which emit blue or yellow or orange or red light are used in the emitting layers. Particular preference is given to three-layer systems, i.e. systems having three emitting layers, where at least one of these layers preferably comprises at least one compound of the formula (I), (II) or (III) and where the three layers exhibit blue, green and orange or red emission (for the basic structure see, for example, WO 2005/011013). The compounds according to the invention may alternatively and/or additionally also be present in the electron-transport layer or in another layer.

It should be noted that, for the generation of white light, an emitter compound used individually which emits in a broad wavelength range may also be suitable instead of a plurality of emitter compounds emitting in colour.

It is preferred in accordance with the invention if the compound of the formula (I), (II) or (III) is employed in an electronic device comprising one or more phosphorescent dopants. The compound can be used in various layers here, preferably in an electron-transport layer or in an emitting layer.

In accordance with the present application, the term phosphorescent emitters encompasses compounds in which the light emission takes place through a spin-forbidden transition, for example a transition from an excited triplet state or a state having a higher spin quantum number, such as a quintet state.

Suitable phosphorescent dopants are, in particular, compounds which emit light, preferably in the visible region, on suitable excitation and in addition contain at least one atom having an atomic number greater than 20, preferably greater than 38 and less than 84, particularly preferably greater than 56 and less than 80. The phosphorescent dopants used are preferably compounds which contain copper, molybdenum, tungsten, rhenium, ruthenium, osmium, rhodium, iridium, palladium, platinum, silver, gold or europium, in particular compounds which contain iridium, platinum or copper.

For the purposes of the present invention, all luminescent iridium, platinum or copper complexes are regarded as phosphorescent compounds.

Examples of phosphorescent dopants are revealed by the applications WO 2000/70655, WO 2001/41512, WO 2002/02714, WO 2002/15645, EP 1191613, EP 1191612, EP 1191614, WO 2005/033244, WO 2005/019373 and US 2005/0258742. In general, all phosphorescent complexes as used in accordance with the prior art for phosphorescent OLEDs and as are known to the person skilled in the art in the area of organic electroluminescent devices are suitable for use in the devices according to the invention. The person skilled in the art will also be able, without inventive step, to employ further phosphorescent complexes in OLEDs in combination with the compounds according to the invention. Further examples of suitable phosphorescent dopants are revealed by the table following in a later section.

In a preferred embodiment of the present invention, the compounds of the formula (I), (II) or (III) are employed as matrix material in an emitting layer in combination with one or more dopants, preferably phosphorescent dopants.

A dopant in a system comprising a matrix material and a dopant is taken to mean the component whose proportion in the mixture is the smaller. Correspondingly, a matrix material in a system comprising a matrix material and a dopant is taken to mean the component whose proportion in the mixture is the larger.

The proportion of the matrix material in the emitting layer is in this case between 50.0 and 99.9% by vol., preferably between 80.0 and 99.5% by vol. and particularly preferably between 92.0 and 99.5% by vol. for fluorescent emitting layers and between 85.0 and 97.0% by vol. for phosphorescent emitting layers.

Correspondingly, the proportion of the dopants is between 0.1 and 50.0% by vol., preferably between 0.5 and 20.0% by vol. and particularly preferably between 0.5 and 8.0% by vol. for fluorescent emitting layers and between 3.0 and 15.0% by vol. for phosphorescent emitting layers.

An emitting layer of an organic electroluminescent device may also comprise systems comprising a plurality of matrix materials (mixed-matrix systems) and/or a plurality of dopants. In this case too, the dopants are generally the materials whose proportion in the system is the smaller and the matrix materials are the materials whose proportion in the system is the larger. In individual cases, however, the proportion of an individual matrix material in the system may be smaller than the proportion of an individual dopant.

In a further preferred embodiment of the invention, the compounds of the formula (I), (II) or (III) are used as a component of mixed-matrix systems. The mixed-matrix systems preferably comprise two or three different matrix materials, particularly preferably two different matrix materials. One of the two materials here is preferably a material having hole-transporting properties and the other material is a material having electron-transporting properties. The two different matrix materials may be present here in a ratio of 1:50 to 1:1, preferably 1:20 to 1:1, particularly preferably 1:10 to 1:1 and very particularly preferably 1:4 to 1:1. Mixed-matrix systems are preferably employed in phosphorescent organic electroluminescent devices. More precise information on mixed-matrix systems is given, inter alia, in the application WO 2010/108579.

The mixed-matrix systems may comprise one or more dopants. The dopant compounds or the dopant compounds together have, in accordance with the invention, a proportion of 0.1 to 50.0% by vol. in the mixture as a whole and preferably a proportion of 0.5 to 20.0% by vol. in the mixture as a whole. Correspondingly, the matrix components together have a proportion of 50.0 to 99.9% by vol. in the mixture is a whole and preferably a proportion of 80.0 to 99.5% by vol. in the mixture as a whole.

Particularly suitable matrix materials which can be used as matrix components of a mixed-matrix system in combination with the compounds according to the invention are selected from the preferred matrix materials for phosphorescent dopants indicated below or the preferred matrix materials for fluorescent dopants, depending on what type of dopant compound is employed in the mixed-matrix system.

Preferred phosphorescent dopants for use in mixed-matrix systems comprising the compounds according to the invention are the phosphorescent dopants shown above and in a following table.

In a further preferred embodiment of the invention, the compound of the formula (I), (II) or (III) is employed as electron-transport material in an electron-transport layer or electron-injection layer or hole-blocking layer. The emitting layer here may comprise fluorescent and/or phosphorescent emitters.

The further functional materials preferably employed in the electronic devices according to the invention are shown below.

The compounds shown in the following table represent particularly suitable phosphorescent dopants.

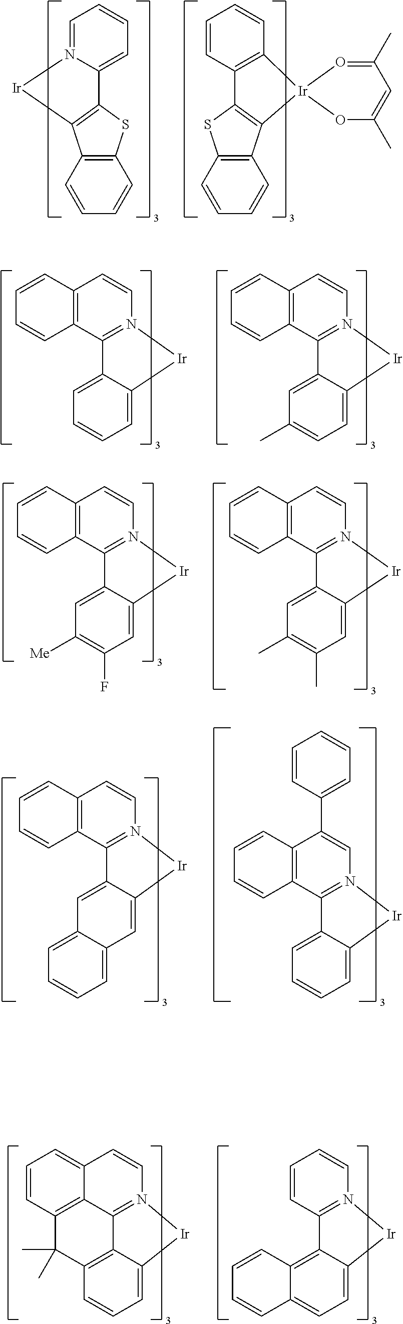

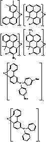

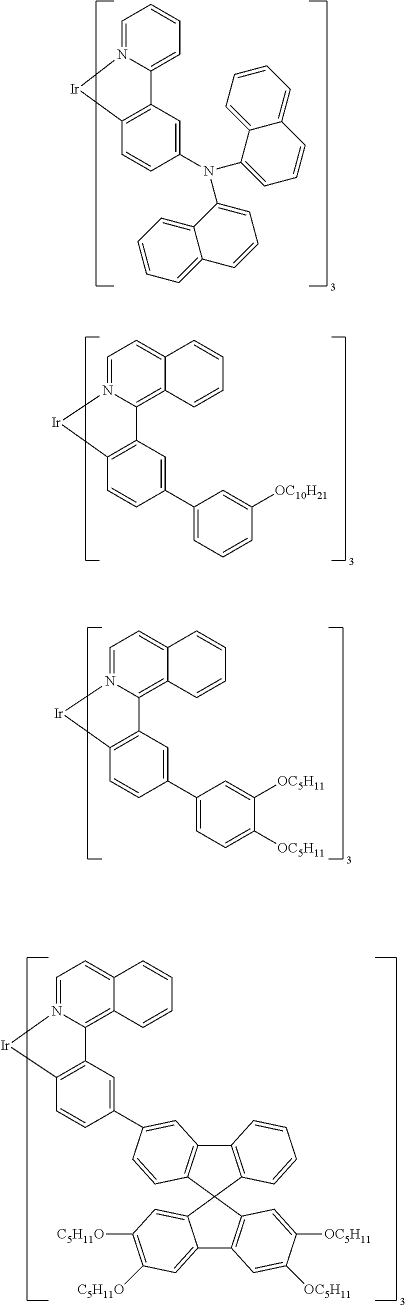

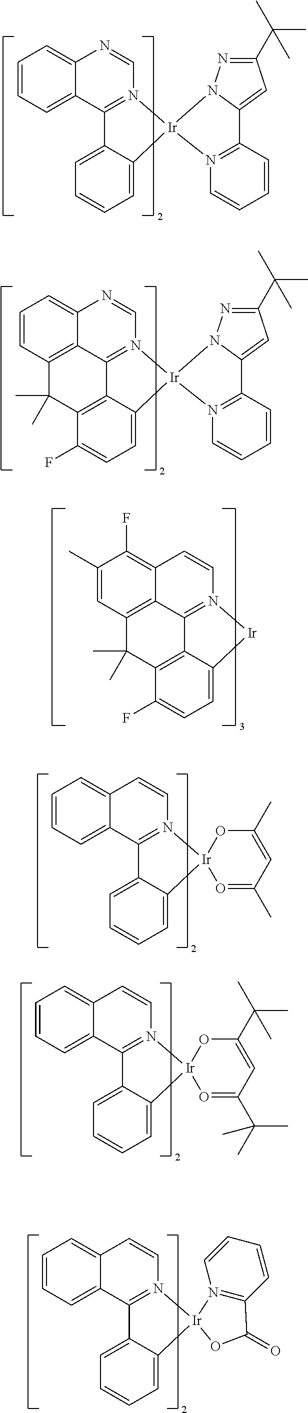

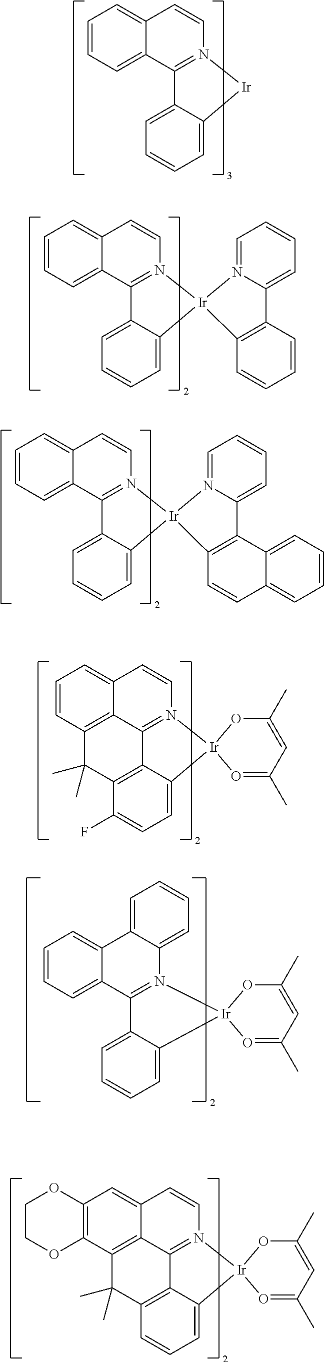

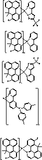

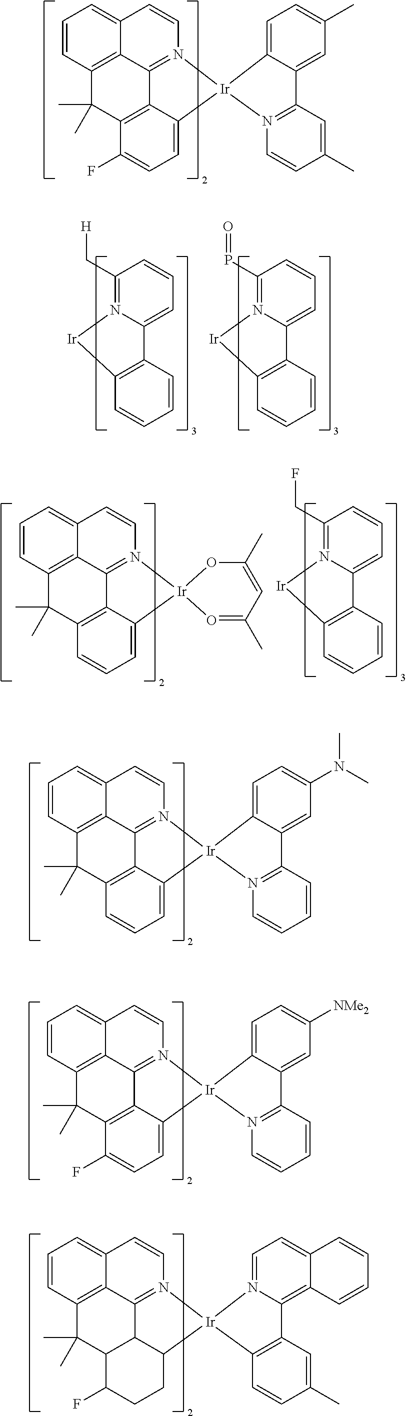

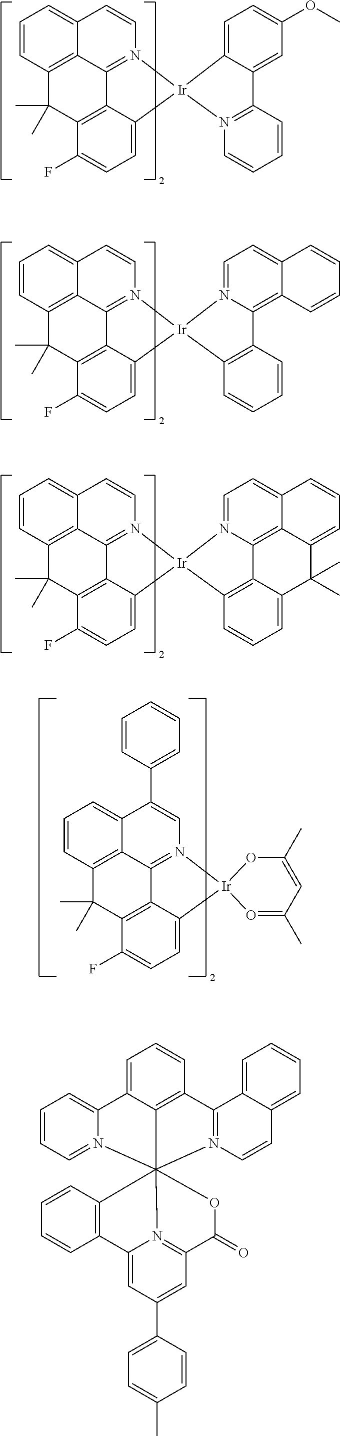

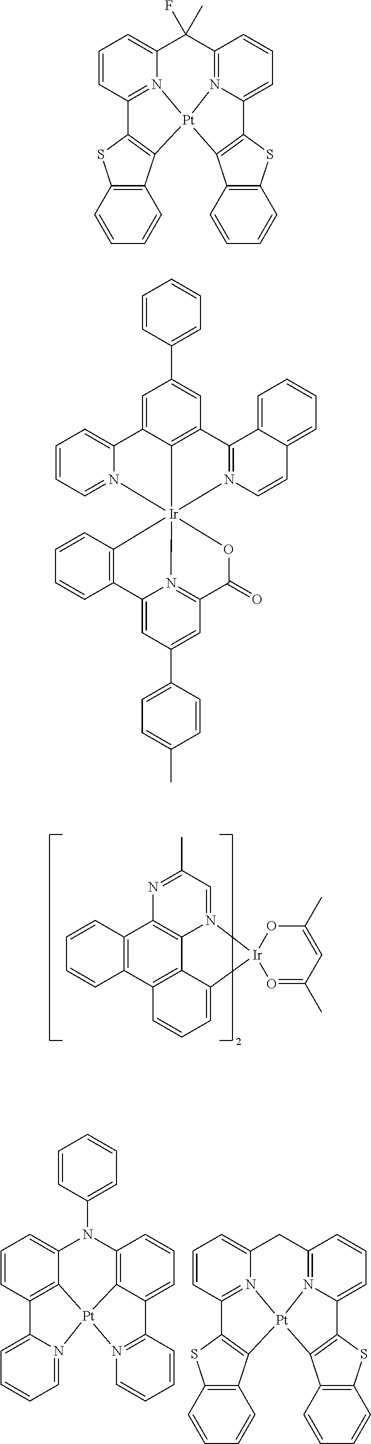

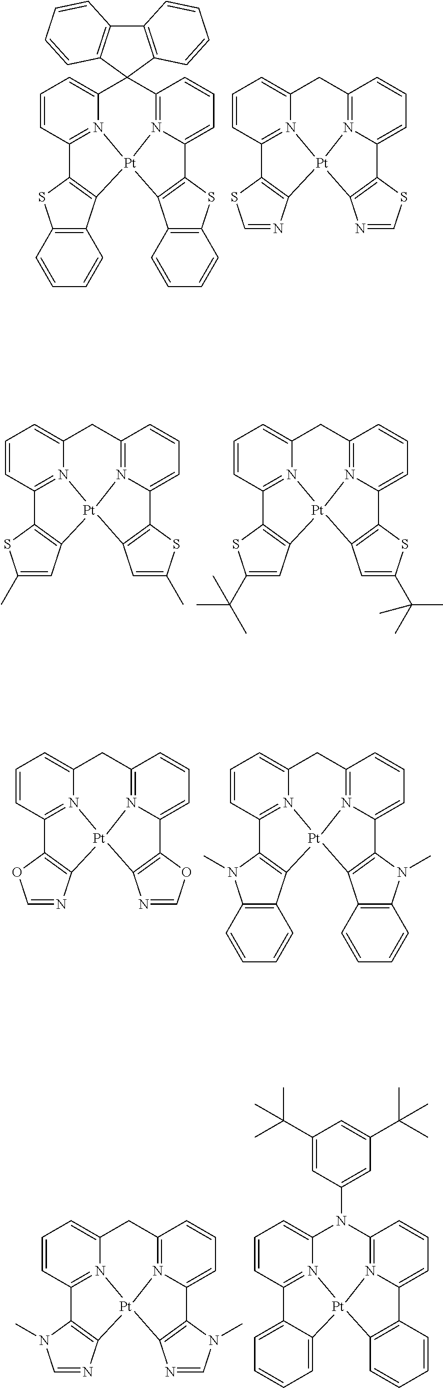

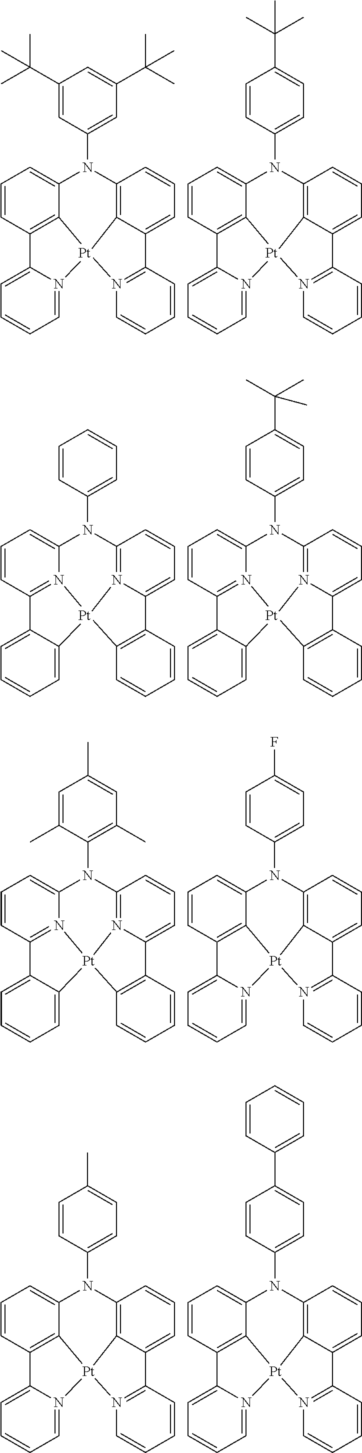

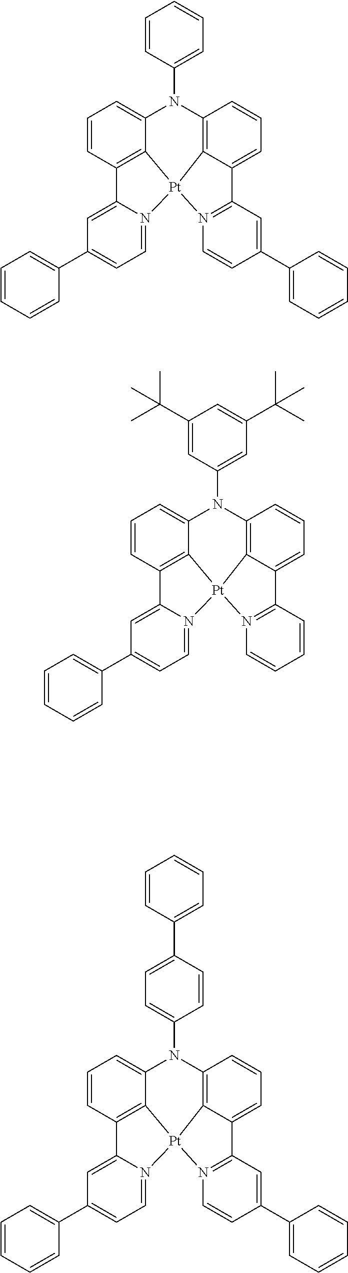

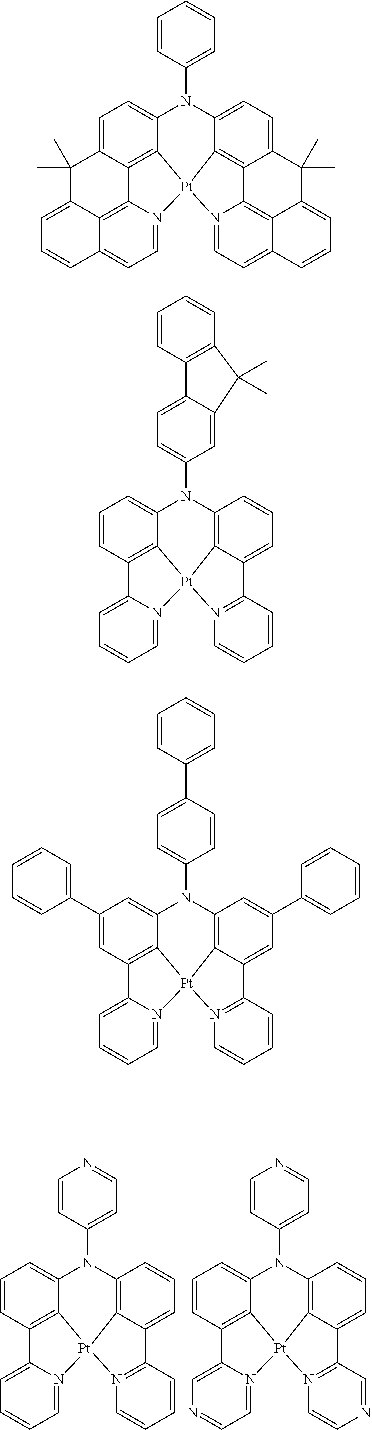

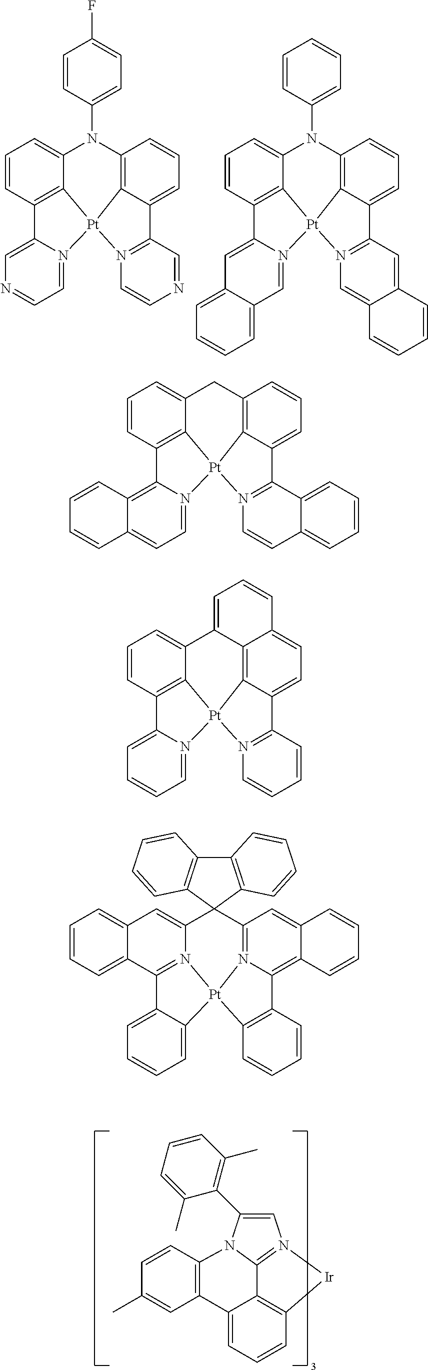

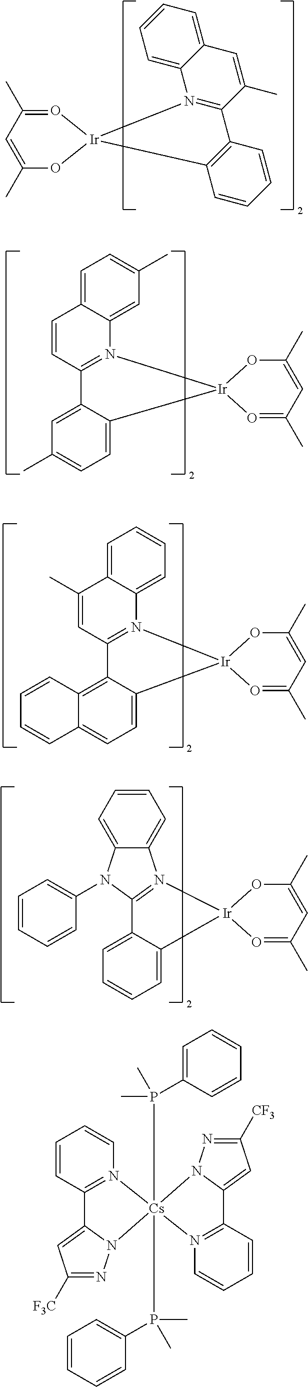

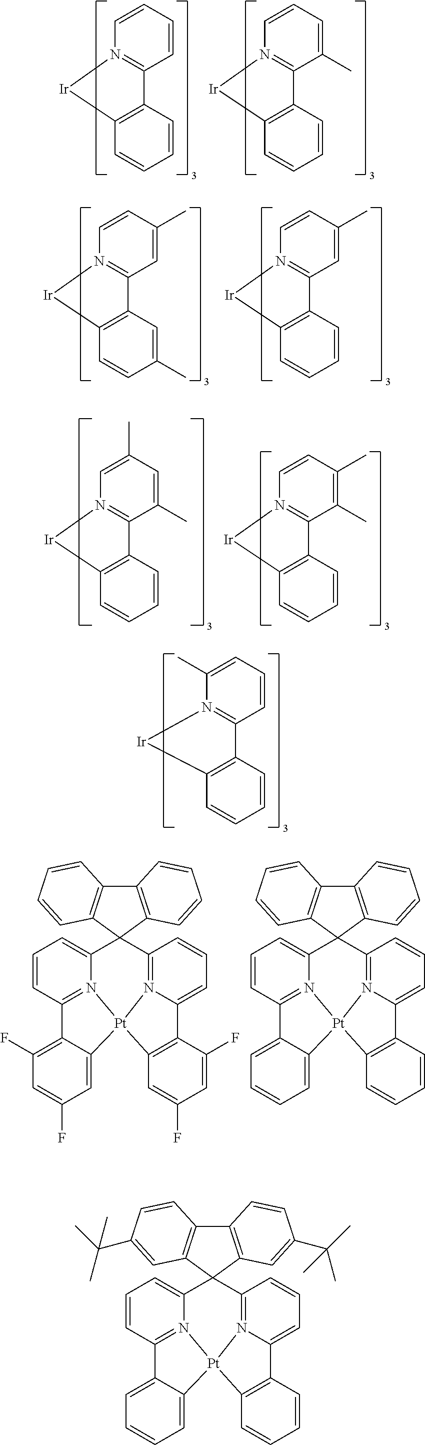

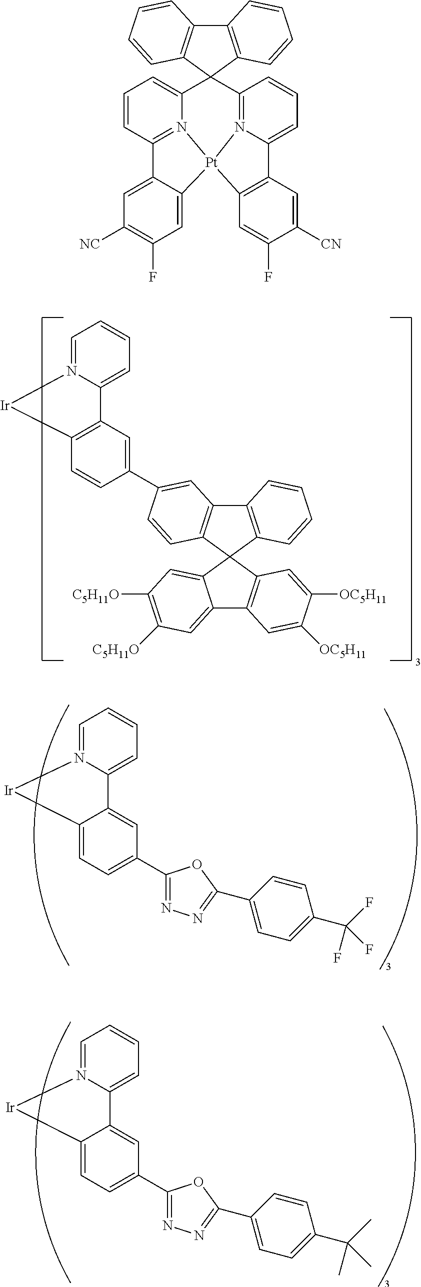

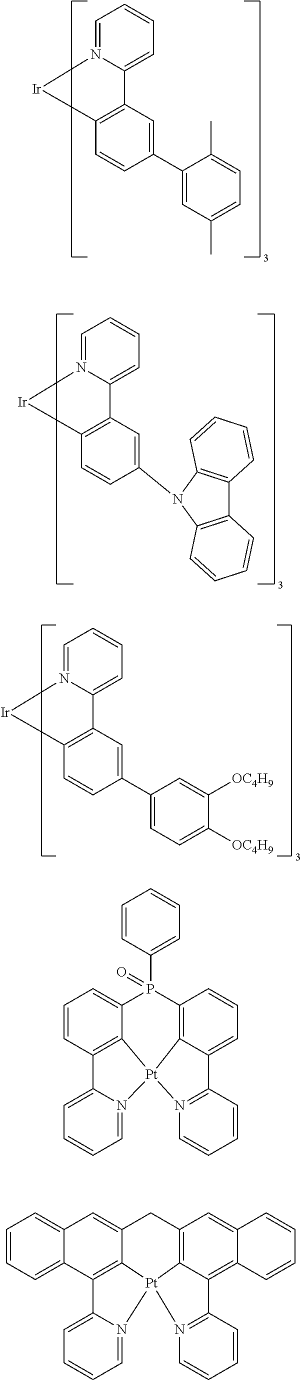

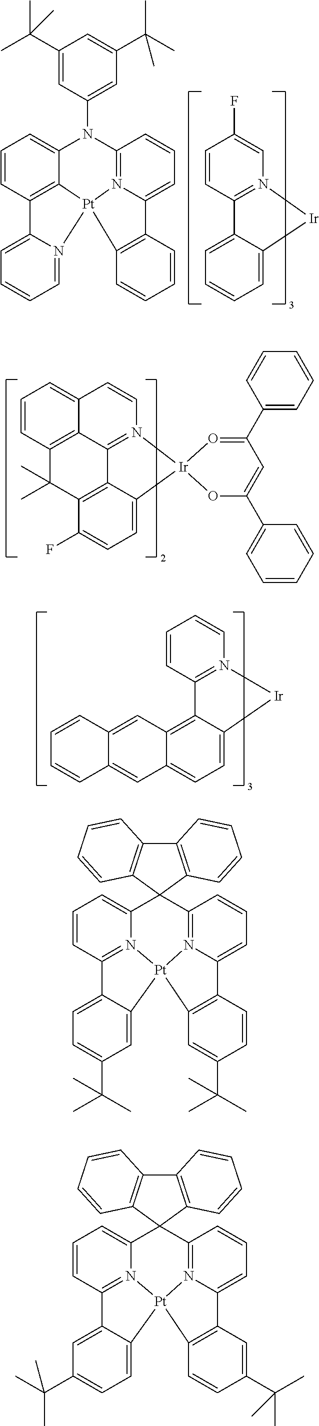

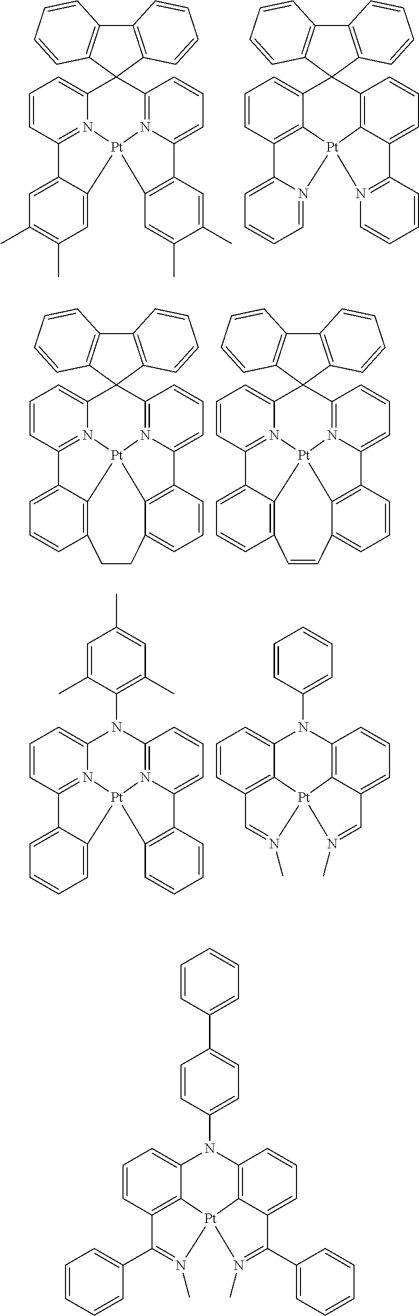

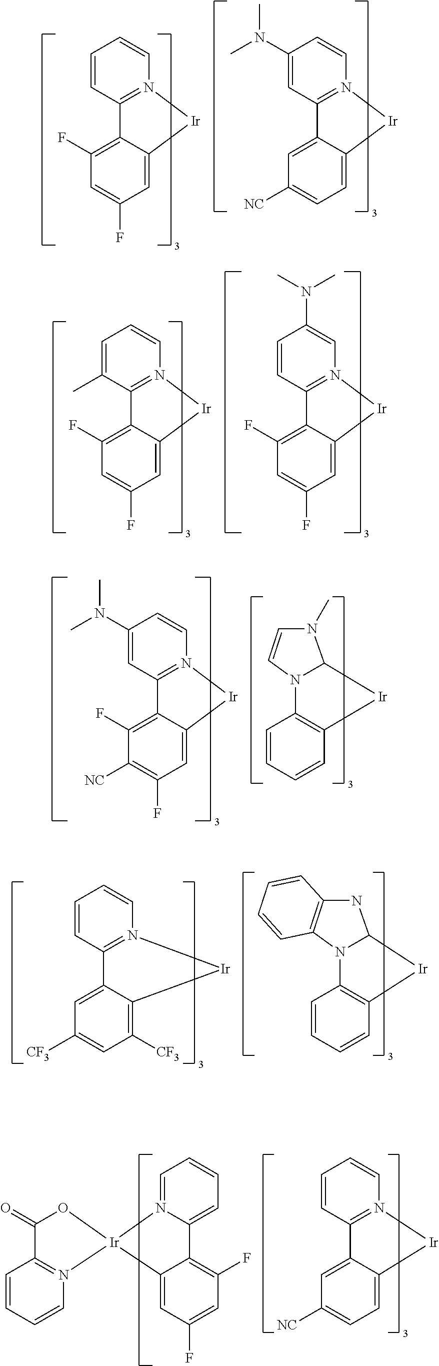

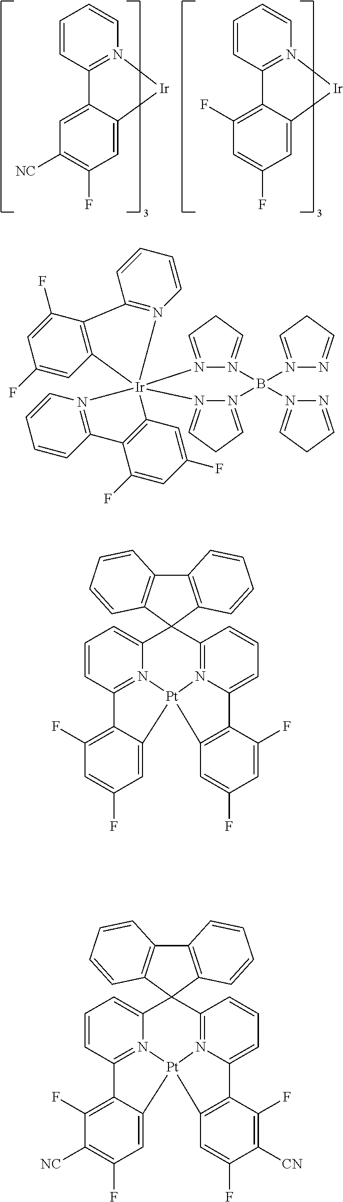

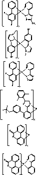

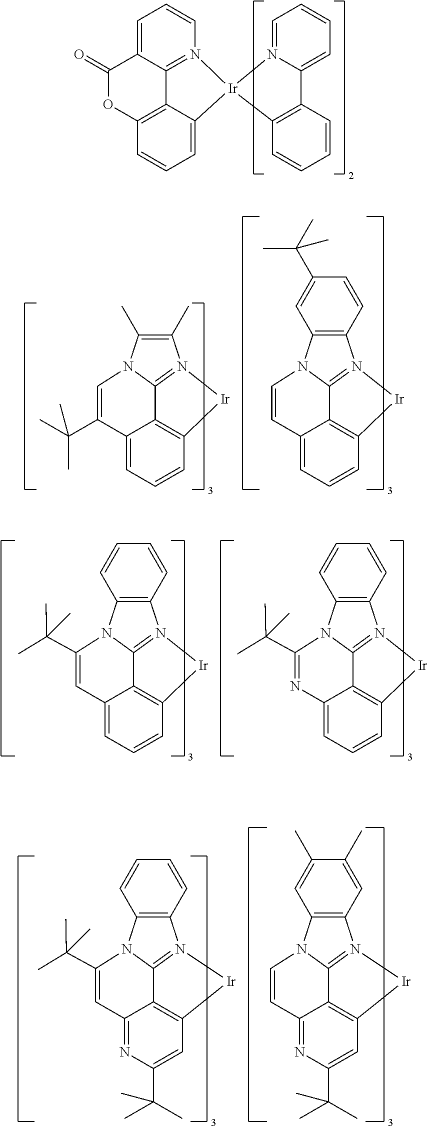

Preferred fluorescent dopants are selected from the class of the arylamines. An arylamine or aromatic amine in the sense of this invention is taken to mean a compound which contains three substituted or unsubstituted aromatic or heteroaromatic ring systems bonded directly to the nitrogen. At least one of these aromatic or heteroaromatic ring systems is preferably a condensed ring system, particularly preferably having at least 14 aromatic ring atoms.

Preferred examples thereof are aromatic anthracenamines, aromatic anthracenediamines, aromatic pyrenamines, aromatic pyrenediamines, aromatic chrysenamines or aromatic chrysenediamines. An aromatic anthracenamine is taken to mean a compound in which one diarylamino group is bonded directly to an anthracene group, preferably in the 9-position. An aromatic anthracenediamine is taken to mean a compound in which two diarylamino groups are bonded directly to an anthracene group, preferably in the 9,10-position. Aromatic pyrenamines, pyrenediamines, chrysenamines and chrysenediamines are defined analogously thereto, where the diarylamino groups are preferably bonded to the pyrene in the 1-position or in the 1,6-position. Further preferred dopants are indenofluorenamines or indeno-fluorenediamines, for example in accordance with WO 2006/108497 or WO 2006/122630, benzoindenofluorenamines or benzoindenofluorene-diamines, for example in accordance with WO 2008/006449, and dibenzoindenofluorenamines or dibenzoindenofluorenediamines, for example in accordance with WO 2007/140847, and the indenofluorene derivatives containing condensed aryl groups which are disclosed in WO 2010/012328. Preference is likewise given to the pyrenarylamines disclosed in WO 2012/048780 and the as yet unpublished EP 12004426.8. Preference is likewise given to the benzoindenofluorenamines disclosed in the as yet unpublished EP 12006239.3.

Suitable matrix materials, preferably for fluorescent emitters, besides the compounds according to the invention, are materials from various classes of substance. Preferred matrix materials are selected from the classes of the oligoarylenes (for example 2,2′,7,7′-tetraphenylspirobifluorene in accordance with EP 676461 or dinaphthylanthracene), in particular the oligoarylenes containing condensed aromatic groups, the oligoarylenevinylenes (for example DPVBi or spiro-DPVBi in accordance with EP 676461), the polypodal metal complexes (for example in accordance with WO 2004/081017), the hole-conducting compounds (for example in accordance with WO 2004/058911), the electron-conducting compounds, in particular ketones, phosphine oxides, sulfoxides, etc. (for example in accordance with WO 2005/084081 and WO 2005/084082), the atropisomers (for example in accordance with WO 2006/048268), the boronic acid derivatives (for example in accordance with WO 2006/117052) or the benzanthracenes (for example in accordance with WO 2008/145239). Particularly preferred matrix materials are selected from the classes of the oligoarylenes, comprising naphthalene, anthracene, benzanthracene and/or pyrene or atropisomers of these compounds, the oligoarylenevinylenes, the ketones, the phosphine oxides and the sulfoxides. Very particularly preferred matrix materials are selected from the classes of the oligoarylenes, comprising anthracene, benzanthracene, benzophenanthrene and/or pyrene or atropisomers of these compounds. An oligoarylene in the sense of this invention is intended to be taken to mean a compound in which at least three aryl or arylene groups are bonded to one another. Preference is furthermore given to the anthracene derivatives disclosed in WO 2006/097208, WO 2006/131192, WO 2007/065550, WO 2007/110129, WO 2007/065678, WO 2008/145239, WO 2009/100925, WO 2011/054442 and EP 1553154, and also the pyrene compounds disclosed in EP 1749809, EP 1905754 and US 2012/0187826.

Preferred matrix materials for phosphorescent emitters, besides the compounds according to the invention, are aromatic ketones, aromatic phosphine oxides or aromatic sulfoxides or sulfones, for example in accordance with WO 2004/013080, WO 2004/093207, WO 2006/005627 or WO 2010/006680, triarylamines, carbazole derivatives, for example CBP (N,N-bis-carbazolylbiphenyl) or the carbazole derivatives disclosed in WO 2005/039246, US 2005/0069729, JP 2004/288381, EP 1205527 or WO 2008/086851, indolocarbazole derivatives, for example in accordance with WO 2007/063754 or WO 2008/056746, indenocarbazole derivatives, for example in accordance with WO 2010/136109, WO 2011/000455 or WO 2013/041176, azacarbazole derivatives, for example in accordance with EP 1617710, EP 1617711, EP 1731584, JP 2005/347160, bipolar matrix materials, for example in accordance with WO 2007/137725, silanes, for example in accordance with WO 2005/111172, azaboroles or boronic esters, for example in accordance with WO 2006/117052, triazine derivatives, for example in accordance with WO 2010/015306, WO 2007/063754 or WO 2008/056746, zinc complexes, for example in accordance with EP 652273 or WO 2009/062578, diazasilole or tetraazasilole derivatives, for example in accordance with WO 2010/054729, diazaphosphole derivatives, for example in accordance with WO 2010/054730, bridged carbazole derivatives, for example in accordance with US 2009/0136779, WO 2010/050778, WO 2011/042107, WO 2011/088877 or WO 2012/143080, triphenylene derivatives, for example in accordance with WO 2012/048781, or lactams, for example in accordance with WO 2011/116865 or WO 2011/137951.

Suitable charge-transport materials, as can be used in the hole-injection or hole-transport layer or in the electron-transport layer of the organic electroluminescent device according to the invention, besides the compounds according to the invention, are, for example, the compounds disclosed in Y. Shirota et al., Chem. Rev. 2007, 107(4), 953-1010, or other materials as are employed in these layers in accordance with the prior art.

Materials which can be used for the electron-transport layer, besides the compounds according to the invention, are all materials as are used in accordance with the prior art as electron-transport materials in the electron-transport layer. Particularly suitable are aluminium complexes, for example Alq₃, zirconium complexes, for example Zrq₄, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives. Furthermore suitable materials are derivatives of the above-mentioned compounds, as disclosed in JP 2000/053957, WO 2003/060956, WO 2004/028217, WO 2004/080975 and WO 2010/072300.

Preferred hole-transport materials which can be used in a hole-transport, hole-injection or electron-blocking layer in the electroluminescent device according to the invention are indenofluorenamine derivatives (for example in accordance with WO 06/122630 or WO 06/100896), the amine derivatives disclosed in EP 1661888, hexaazatriphenylene derivatives (for example in accordance with WO 01/049806), amine derivatives containing condensed aromatic rings (for example in accordance with U.S. Pat. No. 5,061,569), the amine derivatives disclosed in WO 95/09147, monobenzoindenofluorenamines (for example in accordance with WO 08/006449), dibenzoindenofluorenamines (for example in accordance with WO 07/140847), spirobifluorenamines (for example in accordance with WO 2012/034627 or WO 2013/120577), fluorenamines (for example in accordance with the as yet unpublished applications EP 12005369.9, EP 12005370.7 and EP 12005371.5), spirodibenzopyranamines (for example in accordance with WO 2013/083216) and dihydroacridine derivatives (for example in accordance with WO 2012/150001).

The cathode of the organic electroluminescent device preferably comprises metals having a low work function, metal alloys or multilayered structures comprising various metals, such as, for example, alkaline-earth metals, alkali metals, main-group metals or lanthanoids (for example Ca, Ba, Mg, Al, In, Mg, Yb, Sm, etc.). Also suitable are alloys comprising an alkali metal or alkaline-earth metal and silver, for example an alloy comprising magnesium and silver. In the case of multilayered structures, further metals which have a relatively high work function, such as, for example, Ag or Al, can also be used in addition to the said metals, in which case combinations of the metals, such as, for example, Ca/Ag, Mg/Ag or Ag/Ag, are generally used. It may also be preferred to introduce a thin interlayer of a material having a high dielectric constant between a metallic cathode and the organic semiconductor. Suitable for this purpose are, for example, alkali metal fluorides or alkaline-earth metal fluorides, but also the corresponding oxides or carbonates (for example LiF, Li₂O, BaF₂, MgO, NaF, CsF, Cs₂CO₃, etc.). Furthermore, lithium quinolinate (LiQ) can be used for this purpose. The layer thickness of this layer is preferably between 0.5 and 5 nm.

The anode preferably comprises materials having a high work function. The anode preferably has a work function of greater than 4.5 eV vs. vacuum.

Suitable for this purpose are on the one hand metals having a high redox potential, such as, for example, Ag, Pt or Au. On the other hand, metal/metal oxide electrodes (for example AI/Ni/NiO_{R}, AI/PtOₓ) may also be preferred. For some applications, at least one of the electrodes must be transparent or partially transparent in order to facilitate either irradiation of the organic material (organic solar cells) or the coupling-out of light (OLEDs, O-lasers). Preferred anode materials here are conductive mixed metal oxides. Particular preference is given to indium tin oxide (ITO) or indium zinc oxide (IZO). Preference is furthermore given to conductive, doped organic materials, in particular conductive, doped polymers. Furthermore, the anode may also consist of a plurality of layers, for example of an inner layer of ITO and an outer layer of a metal oxide, preferably tungsten oxide, molybdenum oxide or vanadium oxide.

The device is appropriately (depending on the application) structured, pro-vided with contacts and finally sealed, since the lifetime of the devices according to the invention is shortened in the presence of water and/or air.

In a preferred embodiment, the organic electroluminescent device according to the invention is characterised in that one or more layers are coated by means of a sublimation process, in which the materials are applied by vapour deposition in vacuum sublimation units at an initial pressure of less than 10⁻⁵ mbar, preferably less than 10⁻⁶ mbar. However, it is also possible here for the initial pressure to be even lower, for example less than 10⁻⁷ mbar.

Preference is likewise given to an organic electroluminescent device, characterised in that one or more layers are coated by means of the OVPD (organic vapour phase deposition) process or with the aid of carrier-gas sublimation, in which the materials are applied at a pressure of between 10⁻⁵ mbar and 1 bar. A special case of this process is the OVJP (organic vapour jet printing) process, in which the materials are applied directly through a nozzle and are thus structured (for example M. S. Arnold et al., App. Phys. Lett. 2008, 92, 053301).

Preference is furthermore given to an organic electroluminescent device, characterised in that one or more layers are produced from solution, such as, for example, by spin coating, or by means of any desired printing process, such as, for example, screen printing, flexographic printing, nozzle printing or offset printing, but particularly preferably LITI (light induced thermal imaging, thermal transfer printing) or ink-jet printing. Soluble compounds of the formula (I), (II) or (III) are necessary for this purpose. High solubility can be achieved through suitable substitution of the compounds.

For the production of an organic electroluminescent device according to the invention, it is furthermore preferred to apply one or more layers from solution and one or more layers by a sublimation process.

In accordance with the invention, the electronic devices comprising one or more compounds of the formula (I), (II) or (III) can be employed in displays, as light sources in lighting applications and as light sources in medical and/or cosmetic applications (for example light therapy).

### WORKING EXAMPLES

The following working examples serve to illustrate the present invention. They should not be interpreted as being restrictive.

### A) Synthesis Examples

The following syntheses are carried out, unless indicated otherwise, in dried solvents under a protective-gas atmosphere. The compounds according to the invention can be prepared by means of synthetic processes known to the person skilled in the art.

### Example 1

### Precursor

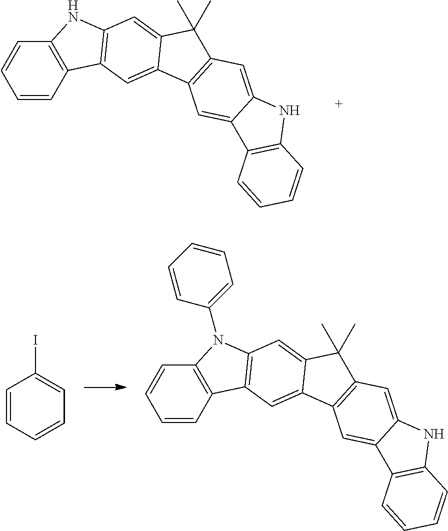 [SMILES: CC1(C)c2cc3[nH]c4ccccc4c3cc2-c2cc3c(cc21)[nH]c1ccccc13 ; Ic1ccccc1 ; CC1(C)c2cc3[nH]c4ccccc4c3cc2-c2cc3c4ccccc4n(-c4ccccc4)c3cc21]

30.2 g (81 mmol) of the compound CAS 1257248-71-7, 18.36 g (90 mmol) of iodobenzene, 22.4 g (162 mmol) of potassium carbonate, 1.84 g (8.1 mmol) of 1,3-di(2-pyridyl)-1,3-propanedione, 1.55 g (8.1 mmol) of copper iodide and 1000 ml of DMF are heated under reflux for 30 h. The solution is subsequently evaporated to dryness in a rotary evaporator. The residue is dissolved in THF and filtered through a short silica-gel bed. The solvent is then removed by means of vacuum. The solid is subsequently recrystallised from heptane/THF and subsequently extracted with hot heptane/toluene over aluminium oxide. The solid which precipitates out on cooling is filtered and dried.

Yield: 19.3 g (43 mmol), 53%

### Example 2a

### Precursor

### 3-(12,12-Dimethyl-12H-10-azaindeno[2,1-b]fluoren-10-yl)phenol

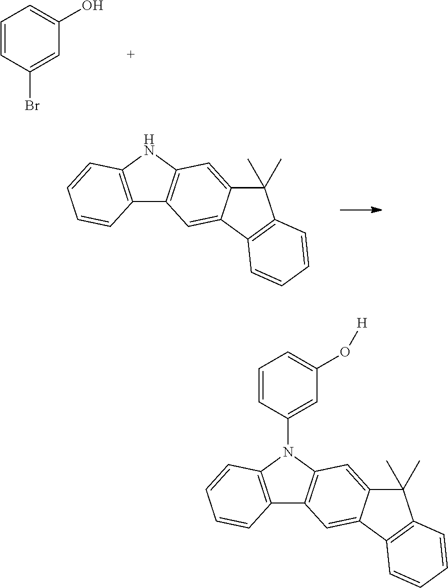 [SMILES: Oc1cccc(Br)c1 ; CC1(C)c2ccccc2-c2cc3c(cc21)[nH]c1ccccc13 ; CC1(C)c2ccccc2-c2cc3c4ccccc4n(-c4cccc(O)c4)c3cc21]

18.5 g (65 mmol) of 12,12-dimethyl-10,12-dihydro-10-azaindeno[2,1-b]fluorene, 21.8 g (85 mmol) of 2-(3-bromophenoxyl)tetrahydropyran and 42.9 g (196 mmol) of potassium phosphate are suspended in 1 I of toluene. 879 mg (3.9 mmol) of palladium(II) acetate and 1.7 ml (6.6 mmol) of tri-tert-butylphosphine are added to this suspension, and the mixture is subsequently stirred at 120° C. for 16 h. After cooling, the organic phase is separated off, filtered through silica gel, washed three times with 200 ml of water and subsequently evaporated to dryness. The solid is subsequently dissolved in 600 ml of THF, and 1 g (5.8 mmol) of p-toluenesulfonic acid is added, and the mixture is stirred at room temperature for 16 h. The mixture is subsequently filtered twice through silica gel with heptane/ethyl acetate 5:1. After evaporation of the solvents, the product precipitates out as white solid. The yield is 17 g (45 mmol; 70%)

The following compounds can be prepared analogously (precursors)

| Starting material 1 | Starting material 2 | Product | Yield |
|---|---|---|---|
| 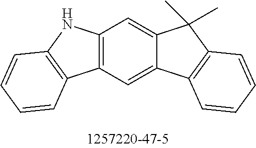 [SMILES: CC1(C)c2ccccc2-c2cc3c(cc21)[nH]c1ccccc13] | 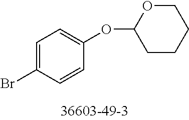 [SMILES: Brc1ccc(OC2CCCCO2)cc1] | 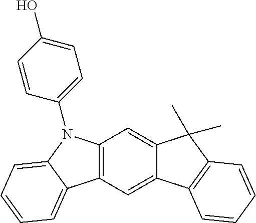 [SMILES: CC1(C)c2ccccc2-c2cc3c4ccccc4n(-c4ccc(O)cc4)c3cc21] | 72% |
| 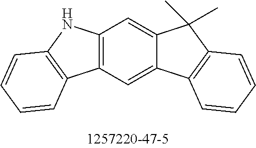 [SMILES: CC1(C)c2ccccc2-c2cc3c(cc21)[nH]c1ccccc13] | 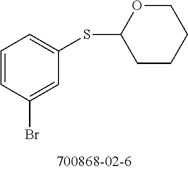 [SMILES: Brc1cccc(SC2CCCCO2)c1] | 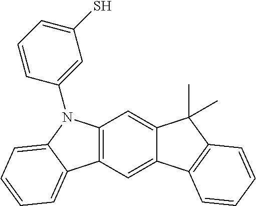 [SMILES: CC1(C)c2ccccc2-c2cc3c4ccccc4n(-c4cccc(S)c4)c3cc21] | 68% |
| 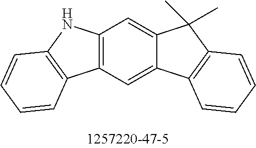 [SMILES: CC1(C)c2ccccc2-c2cc3c(cc21)[nH]c1ccccc13] | 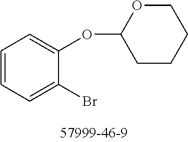 [SMILES: Brc1ccccc1OC1CCCCO1] | 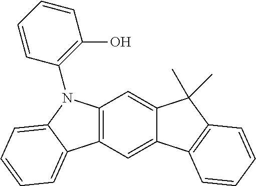 [SMILES: CC1(C)c2ccccc2-c2cc3c4ccccc4n(-c4ccccc4O)c3cc21] | 49% |
| 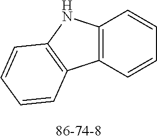 [SMILES: c1ccc2c(c1)[nH]c1ccccc12] | 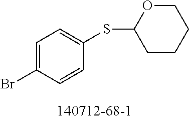 [SMILES: Brc1ccc(SC2CCCCO2)cc1] | 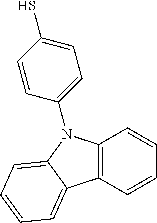 [SMILES: Sc1ccc(-n2c3ccccc3c3ccccc32)cc1] | 73% |
| 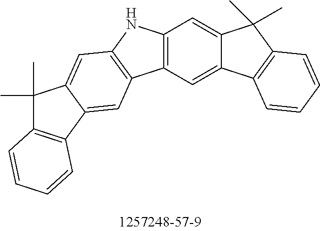 [SMILES: CC1(C)c2ccccc2-c2cc3c(cc21)[nH]c1cc2c(cc13)-c1ccccc1C2(C)C] | 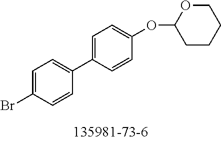 [SMILES: Brc1ccc(-c2ccc(OC3CCCCO3)cc2)cc1] | 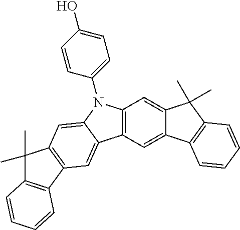 [SMILES: CC1(C)c2ccccc2-c2cc3c4cc5c(cc4n(-c4ccc(O)cc4)c3cc21)C(C)(C)c1ccccc1-5] | 75% |
| 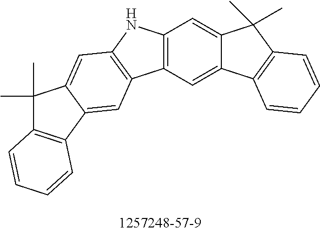 [SMILES: CC1(C)c2ccccc2-c2cc3c(cc21)[nH]c1cc2c(cc13)-c1ccccc1C2(C)C] | 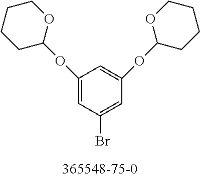 [SMILES: Brc1cc(OC2CCCCO2)cc(OC2CCCCO2)c1] | 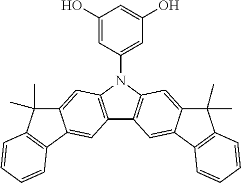 [SMILES: CC1(C)c2ccccc2-c2cc3c4cc5c(cc4n(-c4cc(O)cc(O)c4)c3cc21)C(C)(C)c1ccccc1-5] | 63% |
| 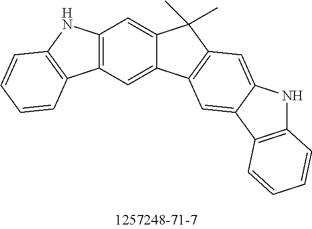 [SMILES: CC1(C)c2cc3[nH]c4ccccc4c3cc2-c2cc3c(cc21)[nH]c1ccccc13] | 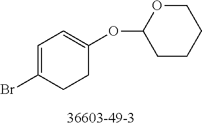 [SMILES: BrC1=CC=C(OC2CCCCO2)CC1] | 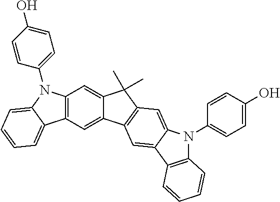 [SMILES: CC1(C)c2cc3c(cc2-c2cc4c5ccccc5n(-c5ccc(O)cc5)c4cc21)c1ccccc1n3-c1ccc(O)cc1] | 62% |
| 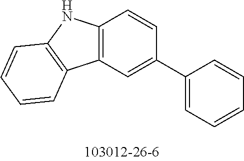 [SMILES: c1ccc(-c2ccc3[nH]c4ccccc4c3c2)cc1] | 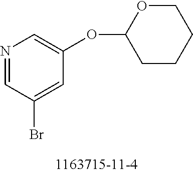 [SMILES: Brc1cncc(OC2CCCCO2)c1] | 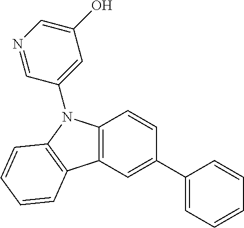 [SMILES: Oc1cncc(-n2c3ccccc3c3cc(-c4ccccc4)ccc32)c1] | 59% |
| 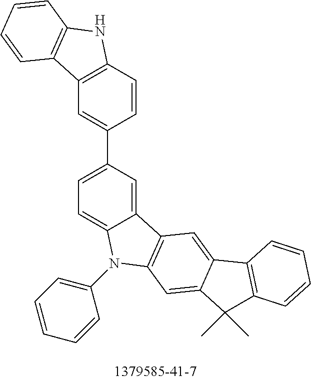 [SMILES: CC1(C)c2ccccc2-c2cc3c4cc(-c5ccc6[nH]c7ccccc7c6c5)ccc4n(-c4ccccc4)c3cc21] | 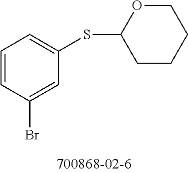 [SMILES: Brc1cccc(SC2CCCCO2)c1] | 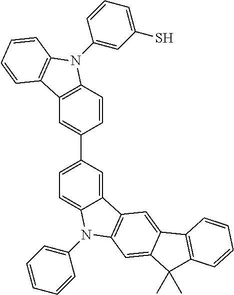 [SMILES: CC1(C)c2ccccc2-c2cc3c4cc(-c5ccc6c(c5)c5ccccc5n6-c5cccc(S)c5)ccc4n(-c4ccccc4)c3cc21] | 61% |
| 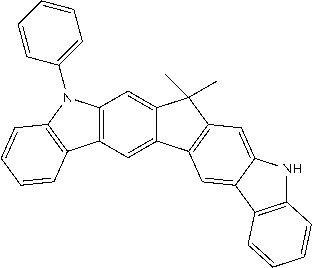 [SMILES: CC1(C)c2cc3[nH]c4ccccc4c3cc2-c2cc3c4ccccc4n(-c4ccccc4)c3cc21] | 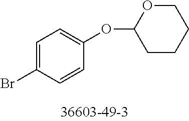 [SMILES: Brc1ccc(OC2CCCCO2)cc1] | 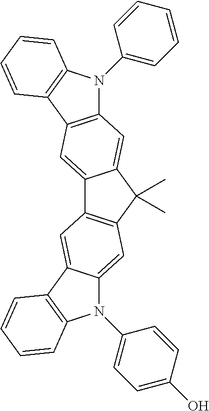 [SMILES: CC1(C)c2cc3c(cc2-c2cc4c5ccccc5n(-c5ccc(O)cc5)c4cc21)c1ccccc1n3-c1ccccc1] | 53% |
| 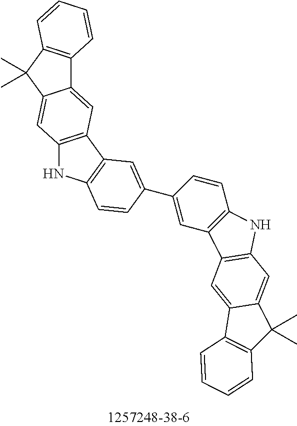 | 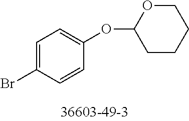 [SMILES: Brc1ccc(OC2CCCCO2)cc1] | 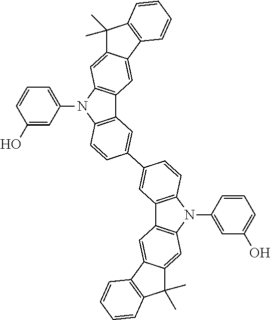 [SMILES: CC1(C)c2ccccc2-c2cc3c4cc(-c5ccc6c(c5)c5cc7c(cc5n6-c5cccc(O)c5)C(C)(C)c5ccccc5-7)ccc4n(-c4cccc(O)c4)c3cc21] | 42% |

### Example 3a

### Compound According to the Invention

### 10-[3-(4,6-Diphenylpyrimidin-2-yloxy)phenyl]-212-dimethyl-10,12-dihydro-10-azaindeno[2,1-b]fluorene

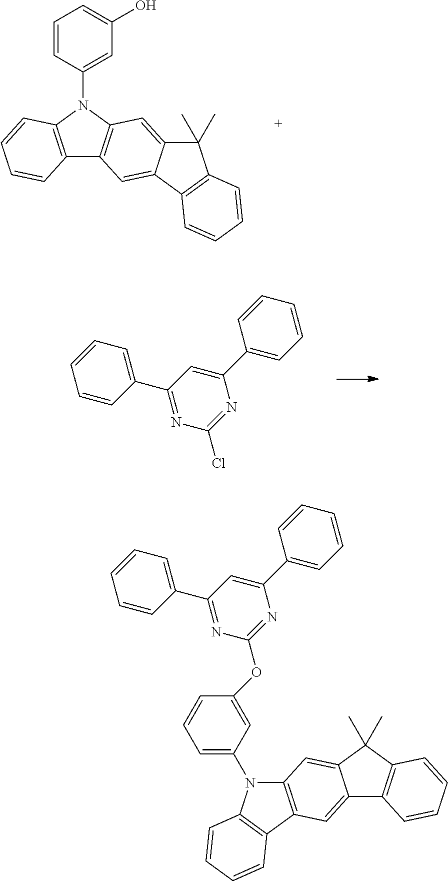 [SMILES: CC1(C)c2ccccc2-c2cc3c4ccccc4n(-c4cccc(O)c4)c3cc21 ; Clc1nc(-c2ccccc2)cc(-c2ccccc2)n1 ; CC1(C)c2ccccc2-c2cc3c4ccccc4n(-c4cccc(Oc5nc(-c6ccccc6)cc(-c6ccccc6)n5)c4)c3cc21]

3.32 g (83 mmol) of sodium hydride are suspended in 200 ml of DMF. 24 g (64 mmol) of 3-(12,12-dimethyl-12H-10-azaindeno[2,1-b]fluoren-10-yl)phenol, dissolved in 100 ml of DMF, are subsequently slowly added via a dropping funnel. When the addition is complete, the mixture is stirred at room pyrimidine, dissolved in 10 ml of anhydrous THF, are slowly added dropwise via a dropping funnel. The mixture is stirred at room temperature for 3 hours until the conversion is complete. The reaction mixture is added to 300 ml of ice and warmed to room temperature with stirring. The solid which precipitates out is filtered and washed with 300 ml of ethanol and 300 ml of n-heptane. The solid is recrystallised from toluene and subsequently sublimed in a high vacuum (3·10⁻⁶ bar). The purity is 99.9% (HPLC). The yield is 8 g (13.2 mmol; 21%)

The following compounds according to the invention can be obtained analogously:

| | Starting material 1 | Starting material 2 | Product | Yield |
|---|---|---|---|---|
| 3b | 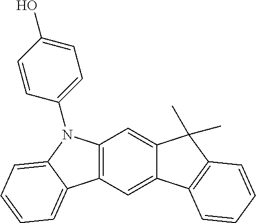 [SMILES: CC1(C)c2ccccc2-c2cc3c4ccccc4n(-c4ccc(O)cc4)c3cc21] | 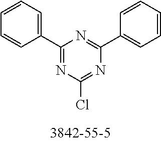 [SMILES: Clc1nc(-c2ccccc2)nc(-c2ccccc2)n1] | 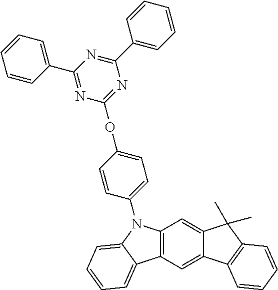 [SMILES: CC1(C)c2ccccc2-c2cc3c4ccccc4n(-c4ccc(Oc5nc(-c6ccccc6)nc(-c6ccccc6)n5)cc4)c3cc21] | 24% |
| 3c | 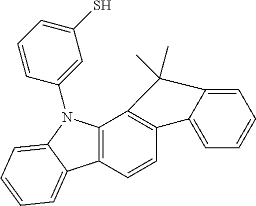 [SMILES: CC1(C)c2ccccc2-c2ccc3c4ccccc4n(-c4cccc(S)c4)c3c21] | 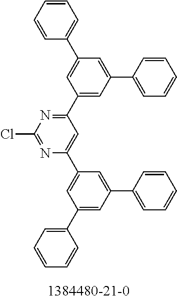 [SMILES: Clc1nc(-c2cc(-c3ccccc3)cc(-c3ccccc3)c2)cc(-c2cc(-c3ccccc3)cc(-c3ccccc3)c2)n1] | 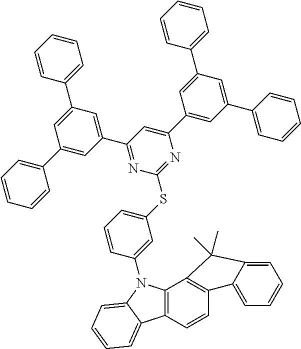 [SMILES: CC1(C)c2ccccc2-c2ccc3c4ccccc4n(-c4cccc(Sc5nc(-c6cc(-c7ccccc7)cc(-c7ccccc7)c6)cc(-c6cc(-c7ccccc7)cc(-c7ccccc7)c6)n5)c4)c3c21] | 19% |
| 3d | 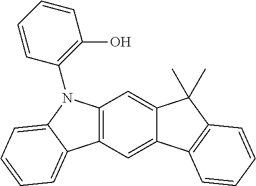 [SMILES: CC1(C)c2ccccc2-c2cc3c4ccccc4n(-c4ccccc4O)c3cc21] | 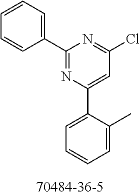 [SMILES: Cc1ccccc1-c1cc(Cl)nc(-c2ccccc2)n1] | 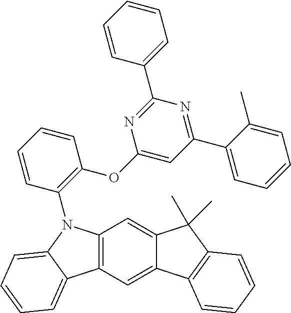 [SMILES: Cc1ccccc1-c1cc(Oc2ccccc2-n2c3ccccc3c3cc4c(cc32)C(C)(C)c2ccccc2-4)nc(-c2ccccc2)n1] | 22% |
| 3e | 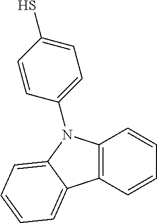 [SMILES: Sc1ccc(-n2c3ccccc3c3ccccc32)cc1] | 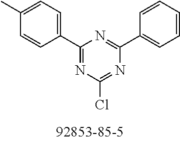 [SMILES: Cc1ccc(-c2nc(Cl)nc(-c3ccccc3)n2)cc1] | 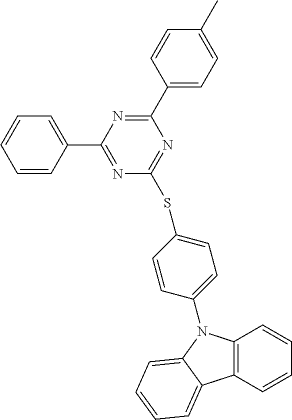 [SMILES: Cc1ccc(-c2nc(Sc3ccc(-n4c5ccccc5c5ccccc54)cc3)nc(-c3ccccc3)n2)cc1] | 25% |
| 3f | 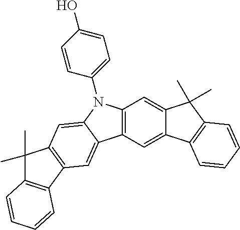 [SMILES: CC1(C)c2ccccc2-c2cc3c4cc5c(cc4n(-c4ccc(O)cc4)c3cc21)C(C)(C)c1ccccc1-5] | 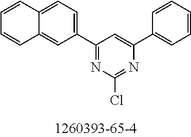 [SMILES: Clc1nc(-c2ccccc2)cc(-c2ccc3ccccc3c2)n1] | 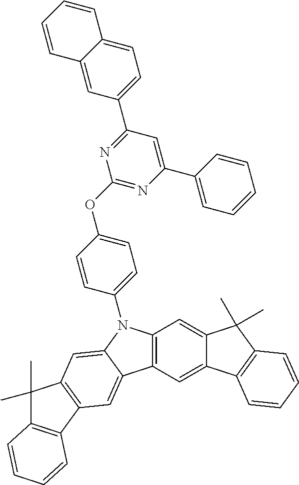 [SMILES: CC1(C)c2ccccc2-c2cc3c4cc5c(cc4n(-c4ccc(Oc6nc(-c7ccccc7)cc(-c7ccc8ccccc8c7)n6)cc4)c3cc21)C(C)(C)c1ccccc1-5] | 23% |
| 3g | 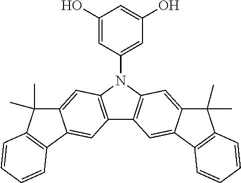 [SMILES: CC1(C)c2ccccc2-c2cc3c4cc5c(cc4n(-c4cc(O)cc(O)c4)c3cc21)C(C)(C)c1ccccc1-5] | 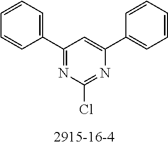 [SMILES: Clc1nc(-c2ccccc2)cc(-c2ccccc2)n1] | 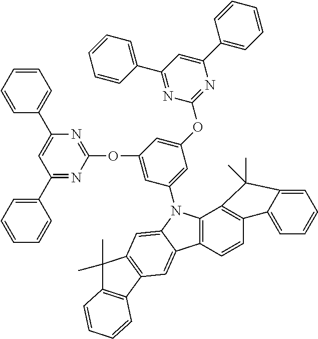 [SMILES: CC1(C)c2ccccc2-c2cc3c4ccc5c(c4n(-c4cc(Oc6nc(-c7ccccc7)cc(-c7ccccc7)n6)cc(Oc6nc(-c7ccccc7)cc(-c7ccccc7)n6)c4)c3cc21)C(C)(C)c1ccccc1-5] | 17% |
| 3h | 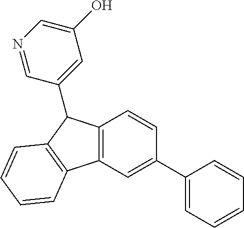 [SMILES: Oc1cncc(C2c3ccccc3-c3cc(-c4ccccc4)ccc32)c1] | 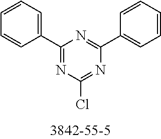 [SMILES: Clc1nc(-c2ccccc2)nc(-c2ccccc2)n1] | 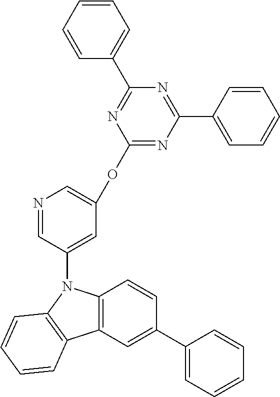 [SMILES: c1ccc(-c2ccc3c(c2)c2ccccc2n3-c2cncc(Oc3nc(-c4ccccc4)nc(-c4ccccc4)n3)c2)cc1] | 14% |
| 3i | 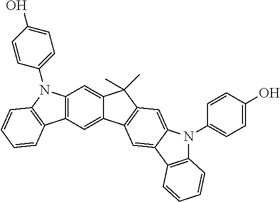 [SMILES: CC1(C)c2cc3c(cc2-c2cc4c5ccccc5n(-c5ccc(O)cc5)c4cc21)c1ccccc1n3-c1ccc(O)cc1] | 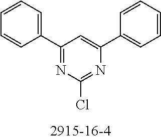 [SMILES: Clc1nc(-c2ccccc2)cc(-c2ccccc2)n1] | 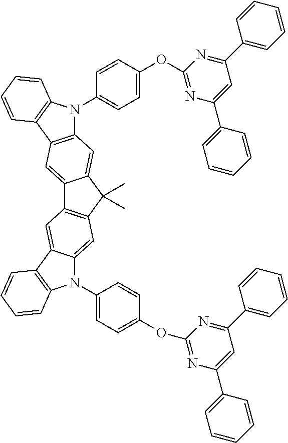 [SMILES: CC1(C)c2cc3c(cc2-c2cc4c5ccccc5n(-c5ccc(Oc6nc(-c7ccccc7)cc(-c7ccccc7)n6)cc5)c4cc21)c1ccccc1n3-c1ccc(Oc2nc(-c3ccccc3)cc(-c3ccccc3)n2)cc1] | 15% |
| 3j | 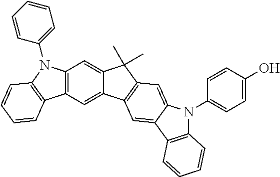 [SMILES: CC1(C)c2cc3c(cc2-c2cc4c5ccccc5n(-c5ccc(O)cc5)c4cc21)c1ccccc1n3-c1ccccc1] | 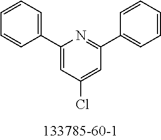 [SMILES: Clc1cc(-c2ccccc2)nc(-c2ccccc2)c1] | 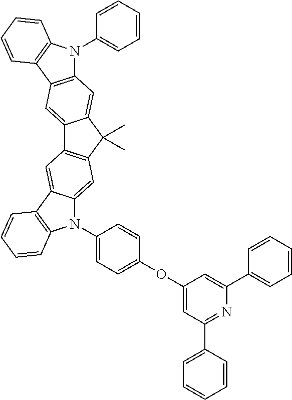 [SMILES: CC1(C)c2cc3c(cc2-c2cc4c5ccccc5n(-c5ccc(Oc6cc(-c7ccccc7)nc(-c7ccccc7)c6)cc5)c4cc21)c1ccccc1n3-c1ccccc1] | 13% |
| 3k | 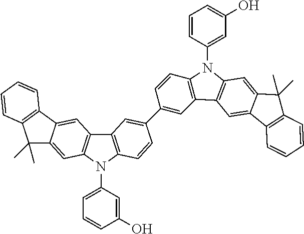 [SMILES: CC1(C)c2ccccc2-c2cc3c4cc(-c5ccc6c(c5)c5cc7c(cc5n6-c5cccc(O)c5)C(C)(C)c5ccccc5-7)ccc4n(-c4cccc(O)c4)c3cc21] | 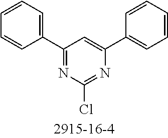 [SMILES: Clc1nc(-c2ccccc2)cc(-c2ccccc2)n1] | 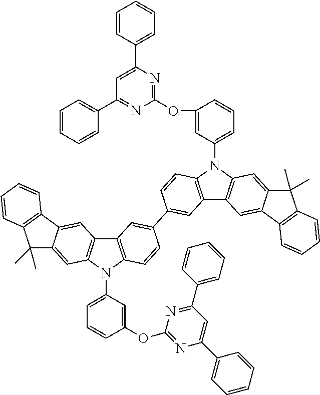 [SMILES: CC1(C)c2ccccc2-c2cc3c4cc(-c5ccc6c(c5)c5cc7c(cc5n6-c5cccc(Oc6nc(-c8ccccc8)cc(-c8ccccc8)n6)c5)C(C)(C)c5ccccc5-7)ccc4n(-c4cccc(Oc5nc(-c6ccccc6)cc(-c6ccccc6)n5)c4)c3cc21] | 12% |

### Example 4a

### Precursor

### 10-(4-Bromophenyl)-12,12-dimethyl-10,12dihydro-10-azaindeno[2,1-b]-fluorene

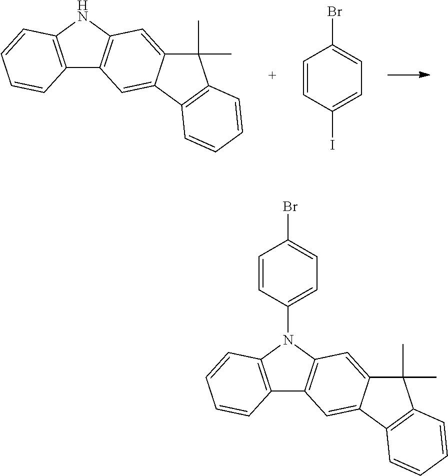 [SMILES: CC1(C)c2ccccc2-c2cc3c(cc21)[nH]c1ccccc13 ; Brc1ccc(I)cc1 ; CC1(C)c2ccccc2-c2cc3c4ccccc4n(-c4ccc(Br)cc4)c3cc21]

23 g (81 mmol) of 12,12-dimethyl-10,12-dihydro-10-azaindeno[2,1-b]fluorene, 115 g (406 mmol) of 1-bromo-4-iodobenzene, 22.4 g (162 mmol) of potassium carbonate, 1.84 g (8.1 mmol) of 1,3-di(2-pyridyl)-1,3-propanedione, 1.55 g (8.1 mmol) of copper iodide and 1000 ml of DMF are heated under reflux for 30 h. The solution is subsequently evaporated to dryness in a rotary evaporator. The residue is dissolved in THF and filtered through a short silica-gel bed. The solvent is then removed by means of vacuum. The solid is subsequently recrystallised from heptane/THF and subsequently extracted with hot heptane/toluene over aluminium oxide. The solid which precipitates out on cooling is filtered and dried.

Yield: 26.3 g (60 mmol), 74%

The following compounds can be obtained analogously (precursors):

| Starting material 1 | Starting material 2 | Product | Yield |
|---|---|---|---|
| 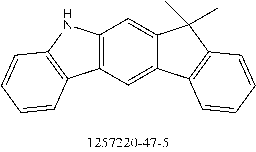 [SMILES: CC1(C)c2ccccc2-c2cc3c(cc21)[nH]c1ccccc13] | 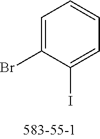 | 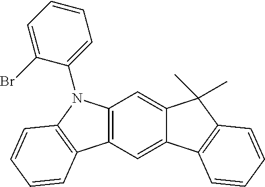 [SMILES: CC1(C)c2ccccc2-c2cc3c4ccccc4n(-c4ccccc4Br)c3cc21] | 61% |
| 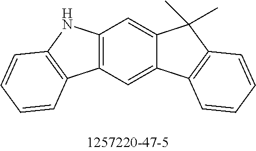 [SMILES: CC1(C)c2ccccc2-c2cc3c(cc21)[nH]c1ccccc13] | 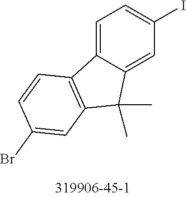 [SMILES: CC1(C)c2cc(Br)ccc2-c2ccc(I)cc21] | 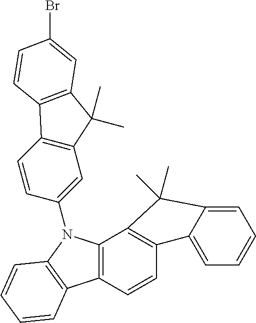 [SMILES: CC1(C)c2cc(Br)ccc2-c2ccc(-n3c4ccccc4c4ccc5c(c43)C(C)(C)c3ccccc3-5)cc21] | 73% |
| 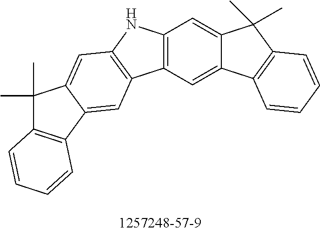 [SMILES: CC1(C)c2ccccc2-c2cc3c(cc21)[nH]c1cc2c(cc13)-c1ccccc1C2(C)C] | 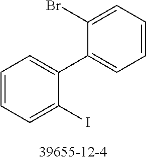 [SMILES: Brc1ccccc1-c1ccccc1I] | 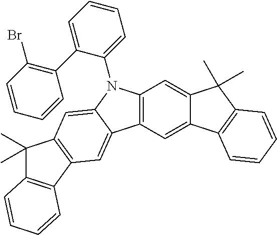 [SMILES: CC1(C)c2ccccc2-c2cc3c4cc5c(cc4n(-c4ccccc4-c4ccccc4Br)c3cc21)C(C)(C)c1ccccc1-5] | 43% |
| 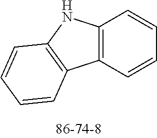 [SMILES: c1ccc2c(c1)[nH]c1ccccc12] | 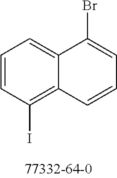 [SMILES: Brc1cccc2c(I)cccc12] | 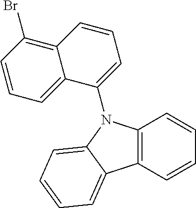 [SMILES: Brc1cccc2c(-n3c4ccccc4c4ccccc43)cccc12] | 58% |
| 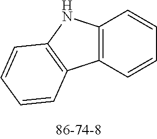 [SMILES: c1ccc2c(c1)[nH]c1ccccc12] | 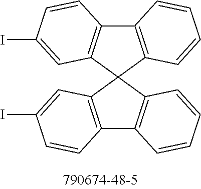 [SMILES: Ic1ccc2c(c1)C1(c3ccccc3-2)c2ccccc2-c2ccc(I)cc21] | 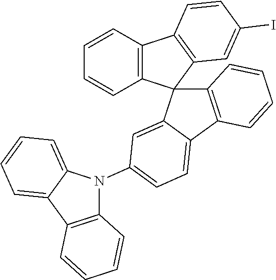 [SMILES: Ic1ccc2c(c1)C1(c3ccccc3-2)c2ccccc2-c2ccc(-n3c4ccccc4c4ccccc43)cc21] | 33% |
| 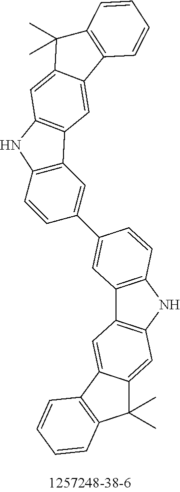 [SMILES: CC1(C)c2ccccc2-c2cc3c(cc21)[nH]c1ccc(-c2ccc4[nH]c5cc6c(cc5c4c2)-c2ccccc2C6(C)C)cc13] | 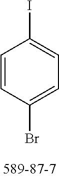 | 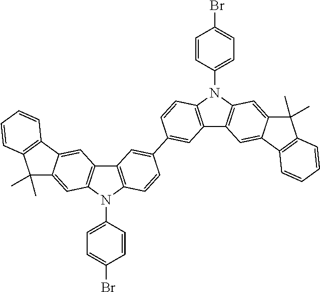 [SMILES: CC1(C)c2ccccc2-c2cc3c4cc(-c5ccc6c(c5)c5cc7c(cc5n6-c5ccc(Br)cc5)C(C)(C)c5ccccc5-7)ccc4n(-c4ccc(Br)cc4)c3cc21] | 46% |

### Example 5a

### precursor

### (4,6-Diphenyl-1,3,5-triazin-2-yl)phenylamine

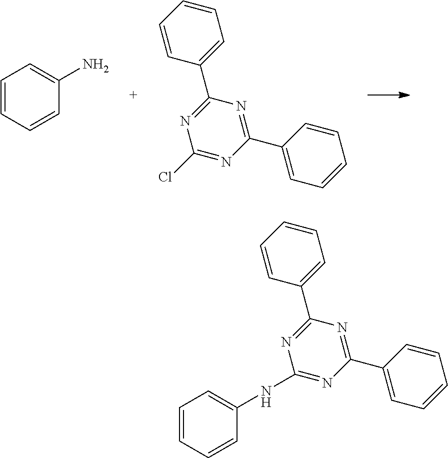

7.76 g (29 mmol) of 2-chloro-4,6-diphenyl-1,3,5-triazine, dissolved in 50 ml of THF, are slowly added dropwise to 2.7 g (29 mmol) of aniline in 180 ml of THF/pyridine, and the mixture is stirred at room temperature. After 20 h, the solvents are removed. The product is obtained as white solid (7.79 g) after precipitation from heptane. This corresponds to a yield of 24 mmol (83%)

The following compounds can be obtained analogously:

| Starting material 1 | Starting material 2 | Product | Yield |
|---|---|---|---|
| 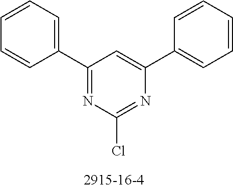 [SMILES: Clc1nc(-c2ccccc2)cc(-c2ccccc2)n1] | 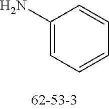 | 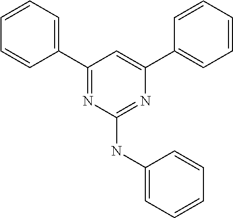 [SMILES: c1ccc(Nc2nc(-c3ccccc3)cc(-c3ccccc3)n2)cc1] | 82% |
| 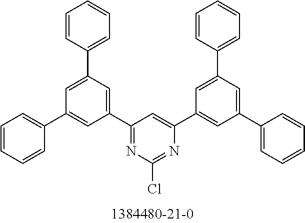 [SMILES: Clc1nc(-c2cc(-c3ccccc3)cc(-c3ccccc3)c2)cc(-c2cc(-c3ccccc3)cc(-c3ccccc3)c2)n1] | 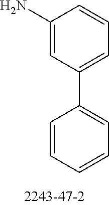 [SMILES: Nc1cccc(-c2ccccc2)c1] | 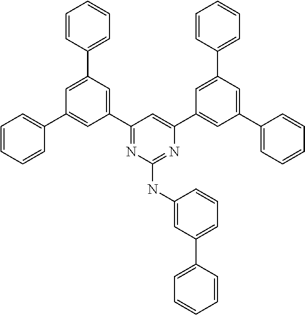 [SMILES: c1ccc(-c2cccc(Nc3nc(-c4cc(-c5ccccc5)cc(-c5ccccc5)c4)cc(-c4cc(-c5ccccc5)cc(-c5ccccc5)c4)n3)c2)cc1] | 65% |
| 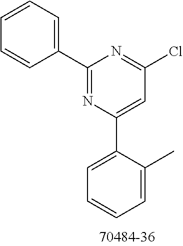 [SMILES: Cc1ccccc1-c1cc(Cl)nc(-c2ccccc2)n1] | 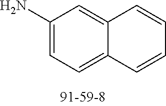 [SMILES: Nc1ccc2ccccc2c1] | 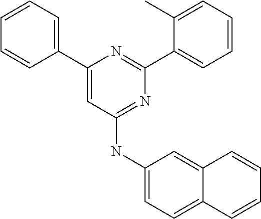 [SMILES: Cc1ccccc1-c1nc(Nc2ccc3ccccc3c2)cc(-c2ccccc2)n1] | 81% |
| 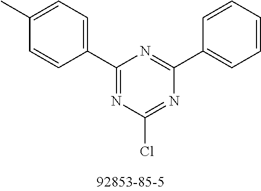 [SMILES: Cc1ccc(-c2nc(Cl)nc(-c3ccccc3)n2)cc1] | 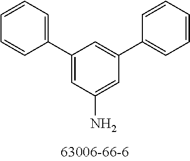 [SMILES: Nc1cc(-c2ccccc2)cc(-c2ccccc2)c1] | 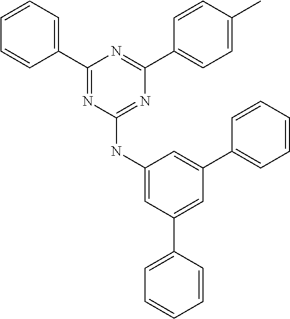 [SMILES: Cc1ccc(-c2nc(Nc3cc(-c4ccccc4)cc(-c4ccccc4)c3)nc(-c3ccccc3)n2)cc1] | 80% |
| 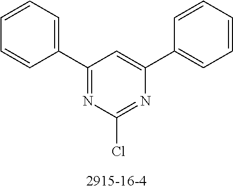 [SMILES: Clc1nc(-c2ccccc2)cc(-c2ccccc2)n1] | 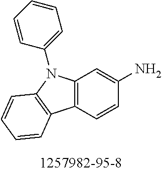 [SMILES: Nc1ccc2c3ccccc3n(-c3ccccc3)c2c1] | 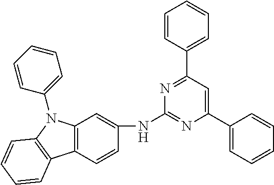 [SMILES: c1ccc(-c2cc(-c3ccccc3)nc(Nc3ccc4c5ccccc5n(-c5ccccc5)c4c3)n2)cc1] | 78% |

### Example 6a

### Compound According to the Invention

### [4-(12,12-Dimethyl-12H-10-azaindeno[2,1-b]fluoren-10-yl)phenyl]-(4,6-diphenyl-1,3,5-triazin-2-yl)phenylamine

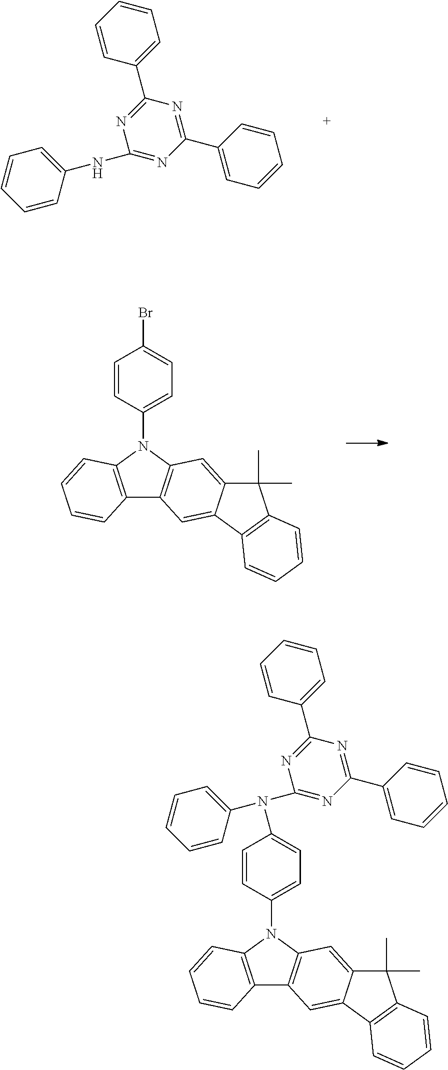 [SMILES: c1ccc(Nc2nc(-c3ccccc3)nc(-c3ccccc3)n2)cc1 ; CC1(C)c2ccccc2-c2cc3c4ccccc4n(-c4ccc(Br)cc4)c3cc21 ; CC1(C)c2ccccc2-c2cc3c4ccccc4n(-c4ccc(N(c5ccccc5)c5nc(-c6ccccc6)nc(-c6ccccc6)n5)cc4)c3cc21]

13 g (29.7 mmol) of 3a, 9.6 g (29.7 mmol) of 4a, 4.6 g (48 mmol) of sodium tert-butoxide, 0.84 g (3 mmol) of tricyclohexylamine, 337 mg (1.5 mmol) of palladium(II) acetate and 300 ml of toluene are heated under reflux for 24 h. After cooling, 200 ml of water are added, the mixture is stirred for a further 30 min., the org. phase is separated off, filtered through a short Celite bed, and the solvent is then removed in vacuo. The residue is recrystallised a number of times from toluene/heptane and finally subjected to fractional sublimation twice (p about 10-6 mbar, T=330-340° C.).

Yield: 6.3 g (9.2 mmol), 31%; purity: 99.9% according to HPLC.

The following compounds according to the invention are obtained analogously:

| | Starting material 1 | Starting material 2 | Product | Yield |
|---|---|---|---|---|
| 6b | 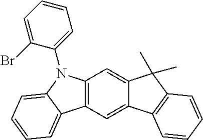 [SMILES: CC1(C)c2ccccc2-c2cc3c4ccccc4n(-c4ccccc4Br)c3cc21] | 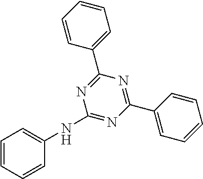 [SMILES: c1ccc(Nc2nc(-c3ccccc3)nc(-c3ccccc3)n2)cc1] | 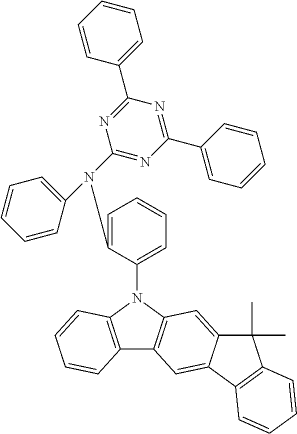 [SMILES: CC1(C)c2ccccc2-c2cc3c4ccccc4n(-c4ccccc4N(c4ccccc4)c4nc(-c5ccccc5)nc(-c5ccccc5)n4)c3cc21] | 32% |
| 6c | 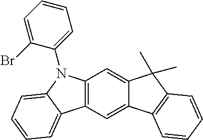 [SMILES: CC1(C)c2ccccc2-c2cc3c4ccccc4n(-c4ccccc4Br)c3cc21] | 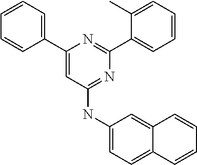 [SMILES: Cc1ccccc1-c1nc(Nc2ccc3ccccc3c2)cc(-c2ccccc2)n1] | 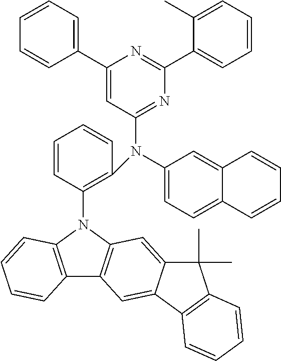 [SMILES: Cc1ccccc1-c1nc(-c2ccccc2)cc(N(c2ccc3ccccc3c2)c2ccccc2-n2c3ccccc3c3cc4c(cc32)C(C)(C)c2ccccc2-4)n1] | 29% |
| 6d | 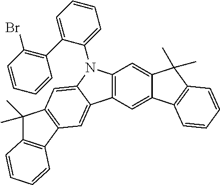 [SMILES: CC1(C)c2ccccc2-c2cc3c4cc5c(cc4n(-c4ccccc4-c4ccccc4Br)c3cc21)C(C)(C)c1ccccc1-5] | 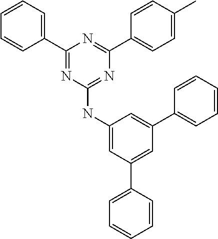 [SMILES: Cc1ccc(-c2nc(Nc3cc(-c4ccccc4)cc(-c4ccccc4)c3)nc(-c3ccccc3)n2)cc1] | 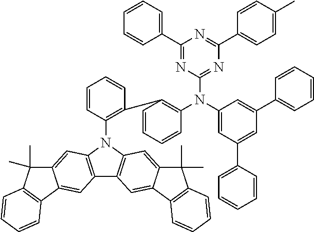 [SMILES: Cc1ccc(-c2nc(-c3ccccc3)nc(N(c3cc(-c4ccccc4)cc(-c4ccccc4)c3)c3ccccc3-c3ccccc3-n3c4cc5c(cc4c4cc6c(cc43)C(C)(C)c3ccccc3-6)-c3ccccc3C5(C)C)n2)cc1] | 12% |
| 6e | 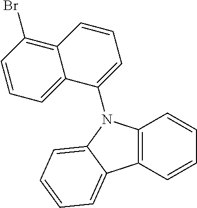 [SMILES: Brc1cccc2c(-n3c4ccccc4c4ccccc43)cccc12] | 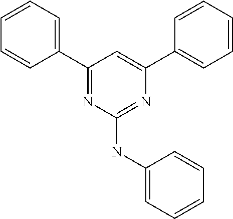 [SMILES: c1ccc(Nc2nc(-c3ccccc3)cc(-c3ccccc3)n2)cc1] | 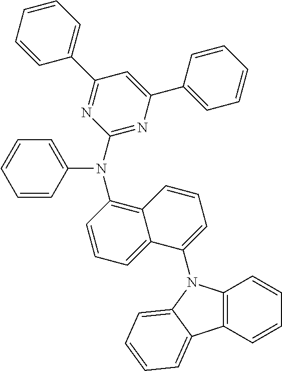 [SMILES: c1ccc(-c2cc(-c3ccccc3)nc(N(c3ccccc3)c3cccc4c(-n5c6ccccc6c6ccccc65)cccc34)n2)cc1] | 28% |
| 6f | 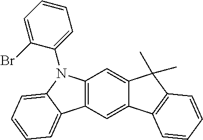 [SMILES: CC1(C)c2ccccc2-c2cc3c4ccccc4n(-c4ccccc4Br)c3cc21] | 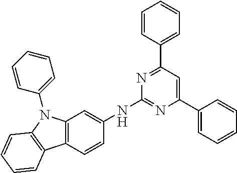 [SMILES: c1ccc(-c2cc(-c3ccccc3)nc(Nc3ccc4c5ccccc5n(-c5ccccc5)c4c3)n2)cc1] | 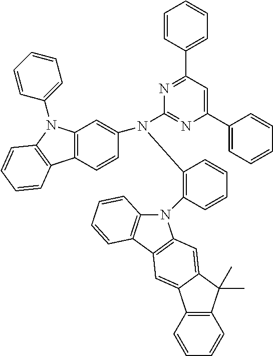 [SMILES: CC1(C)c2ccccc2-c2cc3c4ccccc4n(-c4ccccc4N(c4ccc5c6ccccc6n(-c6ccccc6)c5c4)c4nc(-c5ccccc5)cc(-c5ccccc5)n4)c3cc21] | 17% |
| 6g | 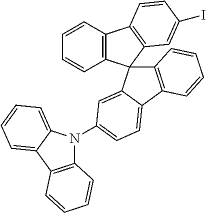 [SMILES: Ic1ccc2c(c1)C1(c3ccccc3-2)c2ccccc2-c2ccc(-n3c4ccccc4c4ccccc43)cc21] | 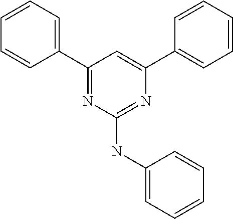 [SMILES: c1ccc(Nc2nc(-c3ccccc3)cc(-c3ccccc3)n2)cc1] | 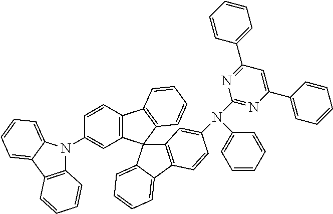 [SMILES: c1ccc(-c2cc(-c3ccccc3)nc(N(c3ccccc3)c3ccc4c(c3)C3(c5ccccc5-4)c4ccccc4-c4ccc(-n5c6ccccc6c6ccccc65)cc43)n2)cc1] | 20% |
| 6h | 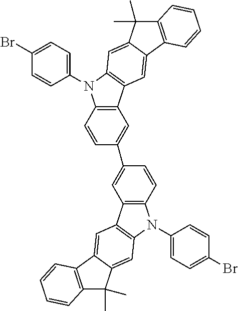 [SMILES: CC1(C)c2ccccc2-c2cc3c4cc(-c5ccc6c(c5)c5cc7c(cc5n6-c5ccc(Br)cc5)C(C)(C)c5ccccc5-7)ccc4n(-c4ccc(Br)cc4)c3cc21] | 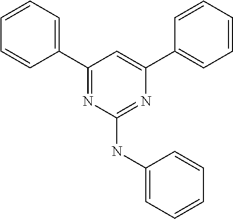 [SMILES: c1ccc(Nc2nc(-c3ccccc3)cc(-c3ccccc3)n2)cc1] | 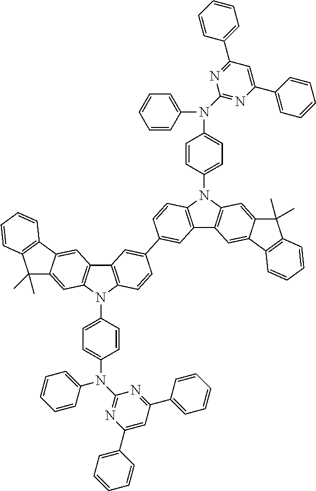 [SMILES: CC1(C)c2ccccc2-c2cc3c4cc(-c5ccc6c(c5)c5cc7c(cc5n6-c5ccc(N(c6ccccc6)c6nc(-c8ccccc8)cc(-c8ccccc8)n6)cc5)C(C)(C)c5ccccc5-7)ccc4n(-c4ccc(N(c5ccccc5)c5nc(-c6ccccc6)cc(-c6ccccc6)n5)cc4)c3cc21] | 13% |

### B) Device Examples

### Production of the OLEDs

The data of various OLEDs are presented in the following examples V1 to E11 (see Tables 1 and 2). Glass plates which have been coated with structured ITO (indium tin oxide) in a thickness of 50 nm are coated with 20 nm of PEDOT:PSS (poly(3,4-ethylenedioxythiophene)poly(styrenesulfonate), purchased as CLEVIOS™ P VP AI 4083 from Heraeus Precious Metals GmbH, Germany, applied by spin coating from aqueous solution) for improved processing. These coated glass plates form the substrates to which the OLEDs are applied.

The OLEDs have in principle the following layer structure: substrate/hole-transport layer (HTL)/interlayer (IL)/electron-blocking layer (EBL)/emission layer (EML)/optional hole-blocking layer (HBL)/electron-transport layer (ETL) and finally a cathode. The cathode is formed by an aluminium layer with a thickness of 100 nm. The precise structure of the OLEDs is shown in Table 1. The materials required for the production of the OLEDs are shown in Table 3.

All materials are applied by thermal vapour deposition in a vacuum chamber.

The emission layer here always consists of at least one matrix material (host material) and an emitting dopant (emitter), which is admixed with the matrix material or matrix materials in a certain proportion by volume by co-evaporation. An expression such as 6 g:IC2:TEG1 (55%:35%:10%) here means that material 6 g is present in the layer in a proportion by volume of 55%, IC2 is present in the layer in a proportion of 35% and TEG1 is present in the layer in a proportion of 10%. Analogously, the electron-transport layer may also consist of a mixture of two materials.

The OLEDs are characterised by standard methods. For this purpose, the electroluminescence spectra, the current efficiency (measured in cd/A), the power efficiency (measured in lm/V) and the external quantum efficiency (EQE, measured in percent) as a function of the luminous density, calculated from current/voltage/luminous density characteristic lines (IUL characteristic lines) assuming Lambert emission characteristics, are determined. The electroluminescence spectra are determined at a luminous density of 1000 cd/m², and the CIE 1931 x and y colour coordinates are calculated therefrom. The term U1000 in Table 2 denotes the voltage required for a luminous density of 1000 cd/m². CE1000 and PE1000 denote the current and power efficiency respectively which are achieved at 1000 cd/m². Finally, EQE1000 denotes the external quantum efficiency at an operating luminous density of 1000 cd/m².

The data obtained for the various OLEDs are summarised in Table 2. Example V1 is a comparative example in accordance with the prior art, Examples E1-11 show data of OLEDs comprising materials according to the invention.

Some of the examples are explained in greater detail below in order to illustrate the advantages of the compounds according to the invention. However, it should be pointed out that this only represents a selection of the data shown in Table 2.

### Use of Compounds According to the Invention as Matrix Materials in Phosphorescent OLEDs

Compounds 3a, 3b, 3c, 3e, 3g, 3i, 6a, 6b, 6e, 6f and 6g according to the invention are employed as matrix materials for phosphorescent emitters in the OLEDs shown in Table 1 (devices E1 to E11). Furthermore, the compound known from the prior art is employed in an analogous function for comparison (device V1).

In general, very good values in relation to lifetime, efficiency and operating voltage are obtained with the compounds according to the invention, both on use in combination with green-emitting triplet emitters and also on use with red-emitting triplet emitters.

For example, excellent performance data are obtained (virtually 17% EQE) in combination with the green-emitting dopant TEG1 with compound 3a as matrix material (Example E2).

A corresponding situation applies to compound 3i as matrix material (Example E6), for which a very low operating voltage was obtained (3.2 V).

In combination with the red-emitting dopant TER1, very good performance data are likewise achieved.

For example, a more than 30% or 25% higher power efficiency respectively is obtained with compound 6e or 6a according to the invention than with compound SdT1 in accordance with the prior art (Examples V1 and E9 or V1 and E8).

TABLE 1
Structure of the OLEDs

| | HTL | IL | EBL | EML | HBL | ETL |
|---|---|---|---|---|---|---|
| Ex. | Thickness | Thickness | Thickness | Thickness | Thickness | Thickness |
| V1 | SpA1 | HATCN | SpMA1 | SdT1:TER1 | — | ST1:LiQ |
| | 90 nm | 5 nm | 130 nm | (92%:8%) | | (50%:50%) |
| | | | | 40 nm | | 40 nm |
| E1 | SpA1 | HATCN | SpMA1 | 3e:TER1 | — | ST1:LiQ |
| | 90 nm | 5 nm | 130 nm | (92%:8%) | | (50%:50%) |
| | | | | 40 nm | | 40 nm |
| E2 | SpA1 | HATCN | SpMA1 | 3a:TEG1 | IC1 | ST1:LiQ |
| | 70 nm | 5 nm | 90 nm | (90%:10%) | 10 nm | (50%:50%) |
| | | | | 30 nm | | 30 nm |
| E3 | SpA1 | HATCN | SpMA1 | 3b:TEG1 | — | ST1:LiQ |
| | 70 nm | 5 nm | 90 nm | (90%:10%) | | (50%:50%) |
| | | | | 30 nm | | 40 nm |
| E4 | SpA1 | HATCN | SpMA1 | 3c:TEG1 | IC1 | ST1:LiQ |
| | 70 nm | 5 nm | 90 nm | (90%:10%) | 10 nm | (50%:50%) |
| | | | | 30 nm | | 30 nm |
| E5 | SpA1 | HATCN | SpMA1 | 3g:TER1 | — | ST1:LiQ |
| | 90 nm | 5 nm | 130 nm | (92%:8%) | | (50%:50%) |
| | | | | 40 nm | | 40 nm |
| E6 | SpA1 | HATCN | SpMA1 | 3i:TEG1 | IC1 | ST1:LiQ |
| | 70 nm | 5 nm | 90 nm | (90%:10%) | 10 nm | (50%:50%) |
| | | | | 30 nm | | 30 nm |
| E7 | SpA1 | HATCN | SpMA1 | 6a:TEG1 | — | ST1:LiQ |
| | 70 nm | 5 nm | 90 nm | (90%:10%) | | (50%:50%) |
| | | | | 30 nm | | 40 nm |
| E8 | SpA1 | HATCN | SpMA1 | 6b:TEG1 | — | ST1:LiQ |
| | 70 nm | 5 nm | 90 nm | (90%:10%) | | (50%:50%) |
| | | | | 30 nm | | 40 nm |
| E9 | SpA1 | HATCN | SpMA1 | 6e:TER1 | — | ST1:LiQ |
| | 90 nm | 5 nm | 130 nm | (92%:8%) | | (50%:50%) |
| | | | | 40 nm | | 40 nm |
| E10 | SpA1 | HATCN | SpMA1 | 6f:IC2:TEG1 | IC1 | ST1:LiQ |
| | 70 nm | 5 nm | 90 nm | (45%:45%:10%) | 10 nm | (50%:50%) |
| | | | | 30 nm | | 30 nm |
| E11 | SpA1 | HATCN | SpMA1 | 6g:IC2:TEG1 | IC1 | ST1:LiQ |
| | 70 nm | 5 nm | 90 nm | (45%:45%:10%) | 10 nm | (50%:50%) |
| | | | | 30 nm | | 30 nm |

TABLE 2
Data of the OLEDs

| | U1000 | CE1000 | PE1000 | EQE | CIE x/y at |
|---|---|---|---|---|---|
| Ex. | (V) | (cd/A) | (lm/W) | 1000 | 1000 cd/m² |
| V1 | 5.3 | 8.7 | 5.2 | 9.4% | 0.67/0.33 |
| E1 | 4.7 | 10 | 7.0 | 11.3% | 0.67/0.33 |
| E2 | 3.7 | 60 | 51 | 16.8% | 0.33/0.62 |
| E3 | 3.3 | 55 | 52 | 15.4% | 0.32/0.62 |
| E4 | 3.6 | 57 | 49 | 15.9% | 0.33/0.62 |
| E5 | 4.9 | 10.3 | 6.6 | 11.1% | 0.67/0.33 |
| E6 | 3.2 | 48 | 47 | 13.6% | 0.33/0.63 |
| E7 | 3.5 | 53 | 47 | 14.7% | 0.33/0.63 |
| E8 | 3.7 | 57 | 48 | 16.1% | 0.33/0.62 |
| E9 | 4.9 | 10.9 | 7.0 | 11.8% | 0.67/0.33 |
| E10 | 3.4 | 49 | 45 | 13.8% | 0.33/0.62 |
| E11 | 3.6 | 51 | 45 | 14.4% | 0.33/0.62 |

TABLE 3
Structural formulae of the materials used in the devices

| | | | |
|---|---|---|---|
| 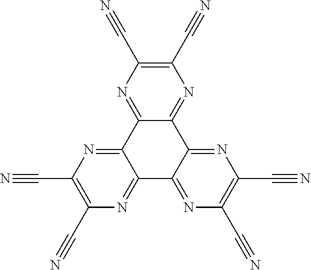 [SMILES: N#Cc1nc2c3nc(C#N)c(C#N)nc3c3nc(C#N)c(C#N)nc3c2nc1C#N] | HATCN | 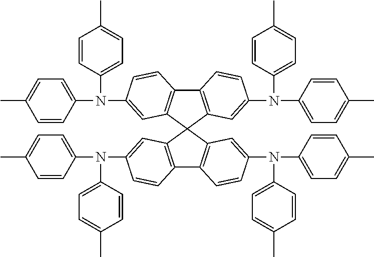 | SpA1 |
| 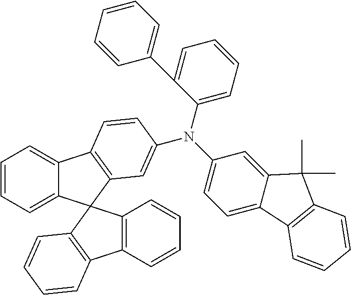 [SMILES: CC1(C)c2ccccc2-c2ccc(N(c3ccc4c(c3)C3(c5ccccc5-c5ccccc53)c3ccccc3-4)c3ccccc3-c3ccccc3)cc21] | SpMA1 | 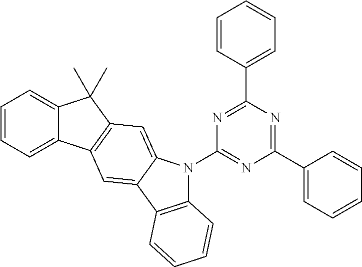 [SMILES: CC1(C)c2ccccc2-c2cc3c4ccccc4n(-c4nc(-c5ccccc5)nc(-c5ccccc5)n4)c3cc21] | IC1 |
| 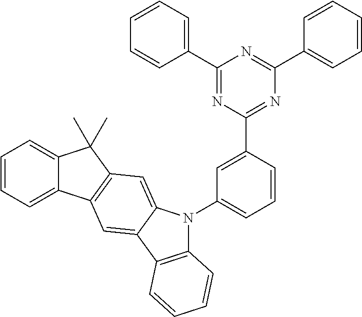 [SMILES: CC1(C)c2ccccc2-c2cc3c4ccccc4n(-c4cccc(-c5nc(-c6ccccc6)nc(-c6ccccc6)n5)c4)c3cc21] | IC2 | 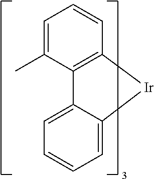 | TEG1 |
| 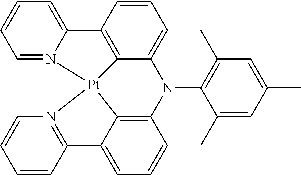 | TER1 | 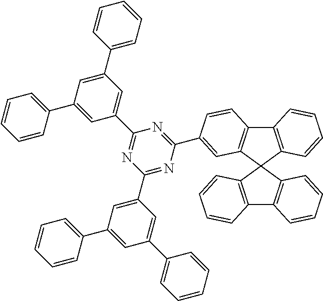 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1] | ST1 |
| 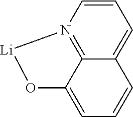 | LiQ | 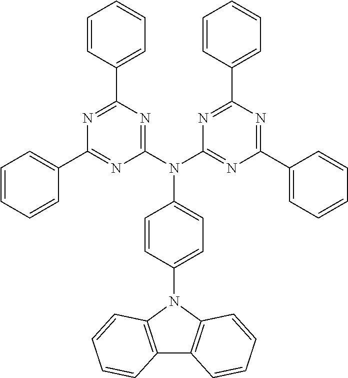 [SMILES: c1ccc(-c2nc(-c3ccccc3)nc(N(c3ccc(-n4c5ccccc5c5ccccc54)cc3)c3nc(-c4ccccc4)nc(-c4ccccc4)n3)n2)cc1] | SdT1 |
| 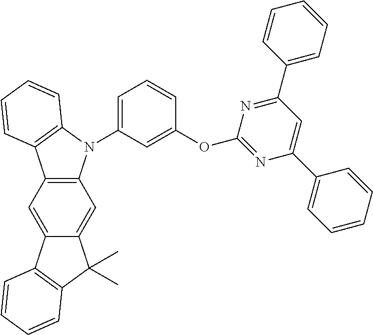 [SMILES: CC1(C)c2ccccc2-c2cc3c4ccccc4n(-c4cccc(Oc5nc(-c6ccccc6)cc(-c6ccccc6)n5)c4)c3cc21] | 3a | 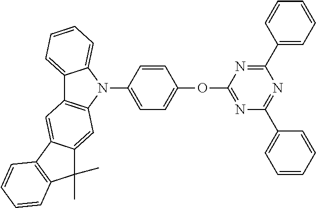 [SMILES: CC1(C)c2ccccc2-c2cc3c4ccccc4n(-c4ccc(Oc5nc(-c6ccccc6)nc(-c6ccccc6)n5)cc4)c3cc21] | 3b |
| 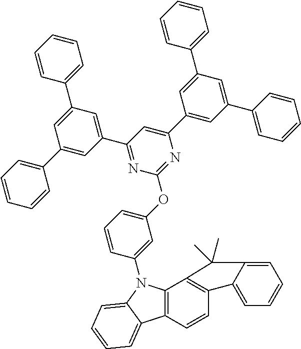 [SMILES: CC1(C)c2ccccc2-c2ccc3c4ccccc4n(-c4cccc(Oc5nc(-c6cc(-c7ccccc7)cc(-c7ccccc7)c6)cc(-c6cc(-c7ccccc7)cc(-c7ccccc7)c6)n5)c4)c3c21] | 3c | 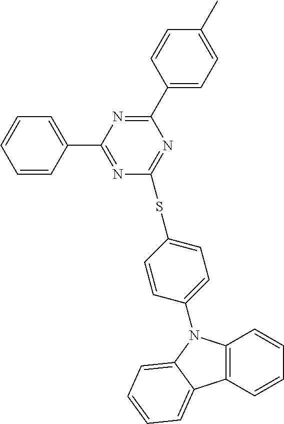 [SMILES: Cc1ccc(-c2nc(Sc3ccc(-n4c5ccccc5c5ccccc54)cc3)nc(-c3ccccc3)n2)cc1] | 3e |
| 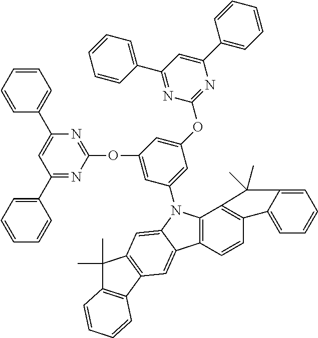 [SMILES: CC1(C)c2ccccc2-c2cc3c4ccc5c(c4n(-c4cc(Oc6nc(-c7ccccc7)cc(-c7ccccc7)n6)cc(Oc6nc(-c7ccccc7)cc(-c7ccccc7)n6)c4)c3cc21)C(C)(C)c1ccccc1-5] | 3g | 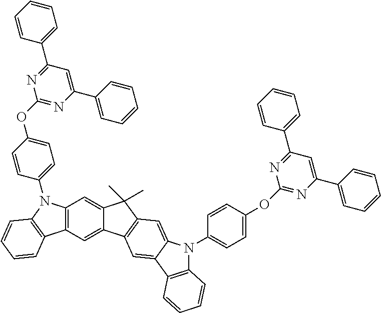 [SMILES: CC1(C)c2cc3c(cc2-c2cc4c5ccccc5n(-c5ccc(Oc6nc(-c7ccccc7)cc(-c7ccccc7)n6)cc5)c4cc21)c1ccccc1n3-c1ccc(Oc2nc(-c3ccccc3)cc(-c3ccccc3)n2)cc1] | 3i |
| 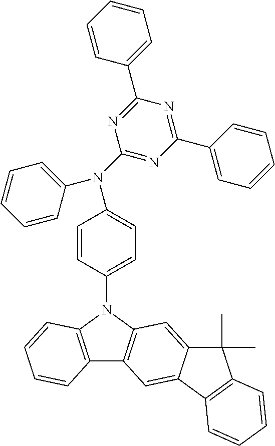 [SMILES: CC1(C)c2ccccc2-c2cc3c4ccccc4n(-c4ccc(N(c5ccccc5)c5nc(-c6ccccc6)nc(-c6ccccc6)n5)cc4)c3cc21] | 6a | 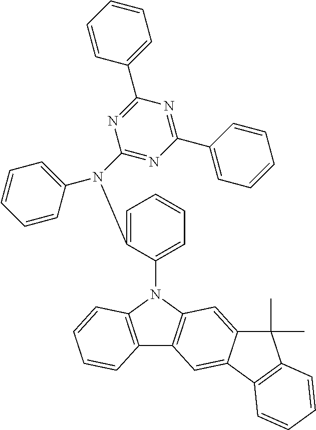 [SMILES: CC1(C)c2ccccc2-c2cc3c4ccccc4n(-c4ccccc4N(c4ccccc4)c4nc(-c5ccccc5)nc(-c5ccccc5)n4)c3cc21] | 6b |
| 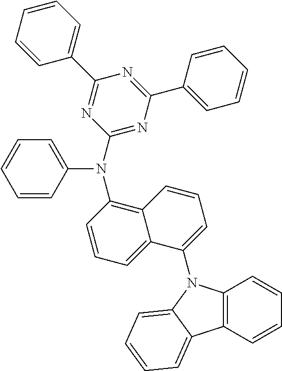 [SMILES: c1ccc(-c2nc(-c3ccccc3)nc(N(c3ccccc3)c3cccc4c(-n5c6ccccc6c6ccccc65)cccc34)n2)cc1] | 6e | 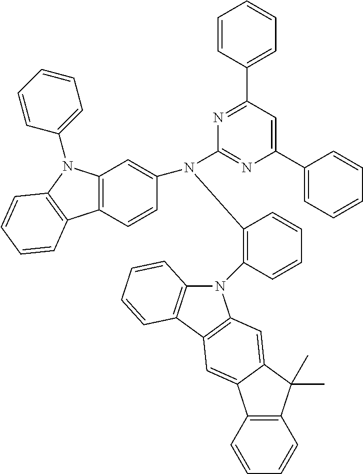 [SMILES: CC1(C)c2ccccc2-c2cc3c4ccccc4n(-c4ccccc4N(c4ccc5c6ccccc6n(-c6ccccc6)c5c4)c4nc(-c5ccccc5)cc(-c5ccccc5)n4)c3cc21] | 6f |
| 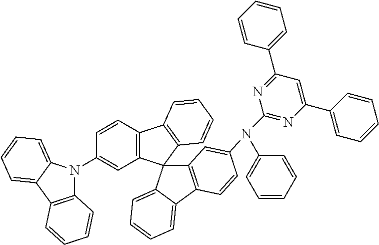 [SMILES: c1ccc(-c2cc(-c3ccccc3)nc(N(c3ccccc3)c3ccc4c(c3)C3(c5ccccc5-4)c4ccccc4-c4ccc(-n5c6ccccc6c6ccccc65)cc43)n2)cc1] | 6g | | |

1.-19. (canceled)

20. A compound of a formula (I), (II) or (III) 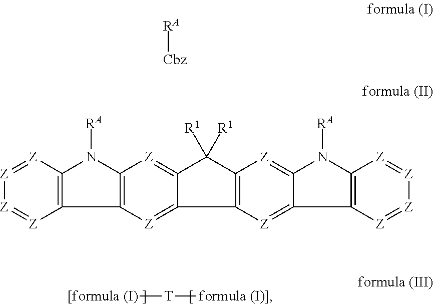
where:
Cbz is a carbazole group which is optionally substituted by one or more radicals R¹ and which is optionally extended by means of one or more condensed-on indeno groups to form an indenocarbazole, and in which one or more aromatic groups ═C(R¹)— or ═C(H)— is optionally replaced by ═N—, and which is bonded to the group R^{A} via the carbazole nitrogen atom;
[formula (I)] is on each occurrence, identically or differently, any desired unit of the formula (I), where the group T is optionally bonded to this unit at any desired position;
Z is on each occurrence, identically or differently, CR¹ or N;
R¹ is on each occurrence, identically or differently, H, D, F, C(═O)R², CN, Si(R²)₃, N(R²)₂, P(═O)(R²)₂, S(═O)R², S(═O)₂R², a straight-chain alkyl or alkoxy group having 1 to 20 C atoms or a branched or cyclic alkyl or alkoxy group having 3 to 20 C atoms or an alkenyl or alkynyl group having 2 to 20 C atoms, where the above-mentioned groups may each be substituted by one or more radicals R² and where one or more CH₂ groups in the above-mentioned groups is optionally replaced by —R²C═CR²—, —C≡C—, Si(R²)₂, C═O, C═NR², —C(═O)O—, —C(═O)NR²—, NR², P(═O)(R²), —O—, —S—, SO or SO₂, or an aromatic or heteroaromatic ring system having 5 to 30 aromatic ring atoms, which may in each case be substituted by one or more radicals R², or an aryloxy or heteroaryloxy group having 5 to 30 aromatic ring atoms, which is optionally substituted by one or more radicals R², where two or more radicals R¹ is optionally linked to one another and may form a ring;
R^{A} is a group of the formula (A) 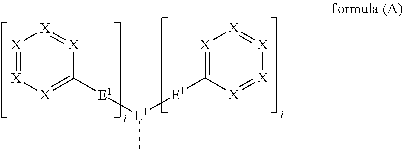
where the dashed line denotes the bond to the remainder of the formula, or R^{A} is equal to R¹, where at least one group R^{A} per formula unit of the formula (I) or (II) conforms to the formula (A);
L¹ is an aromatic or heteroaromatic ring system having 6 to 30 aromatic ring atoms, which is optionally substituted by one or more radicals R¹;
E¹ is on each occurrence, identically or differently, O, S, or NAr¹;
X is on each occurrence, identically or differently, N or CR^{x}, where at least one group X per six-membered ring is equal to N;
i is on each occurrence, identically or differently, 0 or 1, where at least one index i per group of the formula (A) is equal to 1;
R^{x} is on each occurrence, identically or differently, H, D, F, C(═O)R², CN, Si(R²)₃, S(═O)R², S(═O)₂R², a straight-chain alkyl group having 1 to 20 C atoms or a branched or cyclic alkyl group having 3 to 20 C atoms or an alkenyl or alkynyl group having 2 to 20 C atoms, where the above-mentioned groups may each be substituted by one or more radicals R² and where one or more CH₂ groups in the above-mentioned groups is optionally replaced by —R²C═CR²—, —C≡C—, Si(R²)₂, C═O, C═NR², —C(═O)O—, —C(═O)NR²—, NR², P(═O)(R²), —O—, —S—, SO or SO₂, or an aromatic or heteroaromatic ring system having 5 to 30 aromatic ring atoms, which may in each case be substituted by one or more radicals R²;
R² is on each occurrence, identically or differently, H, D, F, C(═O)R³, CN, Si(R³)₃, N(R³)₂, P(═O)(R³)₂, S(═O)R³, S(═O)₂R³, a straight-chain alkyl or alkoxy group having 1 to 20 C atoms or a branched or cyclic alkyl or alkoxy group having 3 to 20 C atoms or an alkenyl or alkynyl group having 2 to 20 C atoms, where the above-mentioned groups may each be substituted by one or more radicals R³ and where one or more CH₂ groups in the above-mentioned groups is optionally replaced by —R³C═CR³—, —C≡C—, Si(R³)₂, C═O, C═NR³, —C(═O)O—, —C(═O)NR³—, NR³, P(═O)(R³), —O—, —S—, SO or SO₂, or an aromatic or heteroaromatic ring system having 5 to 30 aromatic ring atoms, which may in each case be substituted by one or more radicals R³, or an aryloxy or heteroaryloxy group having 5 to 30 aromatic ring atoms, which is optionally substituted by one or more radicals R³, where two or more radicals R² is optionally linked to one another and may form a ring;
R³ is on each occurrence, identically or differently, H, D, F or an aliphatic, aromatic or heteroaromatic organic radical having 1 to 20 C atoms, in which, in addition, one or more H atoms is optionally replaced by D or F; two or more substituents R³ here is optionally linked to one another and form a ring;
Ar¹ is an aromatic ring system having 6 to 30 aromatic ring atoms, which is optionally substituted by one or more radicals R¹;
T is a single bond or an aromatic or heteroaromatic ring system having 6 to 30 aromatic ring atoms, which is optionally substituted by one or more radicals R¹.

21. The compound according to claim 20, wherein the condensation of indeno groups onto the carbazole group in the group Cbz takes place in positions 2 and 3 and/or positions 6 and 7.

22. The compound according to claim 20, wherein the compound contains no condensed aryl or heteroaryl groups having more than 14 aromatic ring atoms.

23. The compound according to claim 20, wherein one index i per formula (A) is equal to one and the other index i is equal to zero.

24. The compound according to claim 20, wherein two or three groups X per six-membered ring are equal to N.

25. The compound according to claim 20, wherein Ar¹ is selected from an aromatic ring system having 6 to 18 aromatic ring atoms, which is optionally substituted by one or more radicals R¹.

26. The compound according to claim 20, wherein groups of the formula (A) conform to one of the following formulae (A-1) to (A-8) 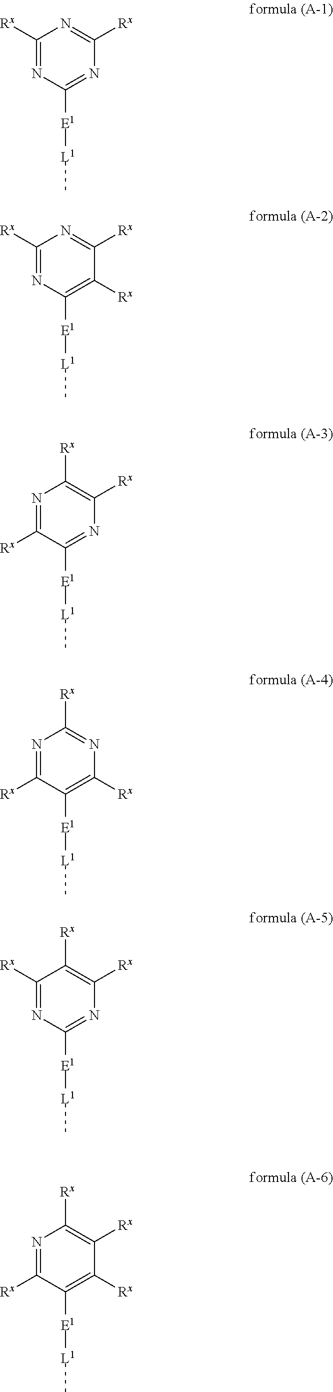 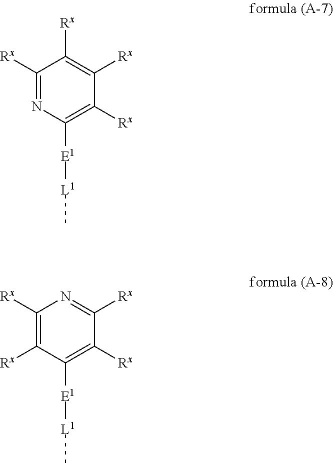
where the groups occurring are as defined in claim 20, and where the dashed line denotes the bond to the remainder of the formula.

27. The compound according to claim 20, wherein E¹ is on each occurrence, identically or differently, O or S.

28. The compound according to claim 20, wherein L¹ is an aromatic ring system having 6 to 24 aromatic ring atoms, which is optionally substituted by one or more radicals R¹.

29. The compound according to claim 20, wherein L¹ contains at least one meta- or ortho-phenylene group, which may optionally be substituted by one or more radicals R¹.

30. The compound according to claim 20, wherein the group Cbz conforms to one of the following formulae (Cbz-1) to (Cbz-3) 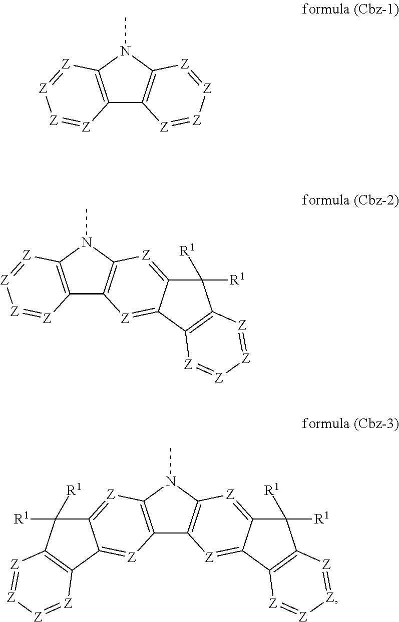
where the dashed line denotes the bond to the group R^{A}, and where the groups occurring are as defined in claim 20.

31. The compound according to claim 20, wherein R^{x} is on each occurrence, identically or differently, H, D, F, CN, a straight-chain alkyl group having 1 to 10 C atoms or a branched or cyclic alkyl group having 3 to 10 C atoms or an alkenyl or alkynyl group having 2 to 10 C atoms, where the above-mentioned groups may each be substituted by one or more radicals R² and where one or more CH₂ groups in the above-mentioned groups is optionally replaced by —R²C═CR²—, —C≡C—, Si(R²)₂ or C═O, or an aromatic or heteroaromatic ring system having 5 to 20 aromatic ring atoms, which may in each case be substituted by one or more radicals R².

32. The compound according to claim 20, wherein the group T represents a single bond.

33. An oligomer, polymer or dendrimer containing one or more compounds according to claim 20, where the bond(s) to the polymer, oligomer or dendrimer is optionally localised at any desired positions in formula (I), (II) or (III) that are substituted by R¹ or R^{x}.

34. A formulation comprising at least one compound according to claim 20 and at least one solvent.

35. A formulation comprising at least one polymer, oligomer or dendrimer according to claim 33 and at least one solvent.

36. An electronic device, selected from the group consisting of an organic integrated circuit (OIC), an organic field-effect transistor (OFET), an organic thin-film transistor (OTFT), an organic light-emitting transistor (OLET), an organic solar cell (OSC), an organic optical detector, an organic photoreceptor, an organic field-quench device (OFQD), an organic light-emitting electrochemical cell (OLEC), an organic laser diode (O-laser) and an organic electroluminescent device (OLED), wherein the device comprises at least one compound according to claim 20.

37. An organic electroluminescent device which comprises anode, cathode and at least one organic layer, where the at least one compound according to claim 20 is employed as matrix material in an emitting layer in combination with one or more dopants, or in that it is employed as electron-transport material in an electron-transport layer, an electron-injection layer or a hole-blocking layer.

38. A process for the preparation of the compound according to claim 20, which comprises employing at least one transition metal-catalysed coupling reaction.
